SEQUENCE LISTING (42 sequences)

SEQ ID NO: 1 - protein, 376 aa, Mus musculus
MEQDLRSIPASKLDKFIENHLPDTSFCADLREVIDALCALLKDRSFRGPVRRMRASKGVKGKGTTLKGRSDADLVVFLNNLTSFEDQLNQQGVLIKEIKKQLCEVQHERRCGVKFEVHSLRSPNSRALSFKLSAPDLLKEVKFDVLPAYDLLDHLNILKKPNQQFYANLISGRTPPGKEGKLSICFMGLRKYFLNCRPTKLKRLIRLVTHWYQLCKEKLGDPLPPQYALELLTVYAWEYGSRVTKFNTAQGFRTVLELVTKYKQLQIYWTVYYDFRHQEVSEYLHQQLKKDRPVILDPADPTRNIAGLNPKDWRRLAGEAAAWLQYPCFKYRDGSSVCSWEVPTEVGVPMKYLLCRIFWLLFWSLFHFIFGKTSSG

SEQ ID NO: 2 - protein, 376 aa, Mus musculus
MEQDLRSIPASKLDKFIENHLPDTSFCADLREVIDALCALLKDRSFRGPVRRMRASKGVKGKGTTLKGRSDADLVVFLNNLTSFEDQLNQQGVLIKEIKKQLCEVQHERRCGVKFEVHSLRSPNSRALSFKLSAPDLLKEVKFDVLPAYDLLDHLNILKKPNQQFYANLISGRTPPGKEGKLSICFMGLRKYFLNCRPTKLKRLIRLVTHWYQLCKEKLGDPLPPQYALELLTVYAWEYGSRVTKFNTAQGFRTVLELVTKYKQLRIYWTVYYDFRHQEVSEYLHQQLKKDRPVILDPADPTRNIAGLNPKDWRRLAGEAATWLQYPCFKYRDGSPVCSWEVPTEVGVPMKYLFCRIFWLLFWSLFHFIFGKTSSG

SEQ ID NO: 3 - protein, 376 aa, Mus musculus
  MISC_FEATURE (36)..(36): X = A or S
  MISC_FEATURE (45)..(45): X = F or S
  MISC_FEATURE (47)..(47): X = Q or F
  MISC_FEATURE (50)..(50): X = G or V
  MISC_FEATURE (63)..(63): X = C or G
  MISC_FEATURE (65)..(65): X = A or T
  MISC_FEATURE (83)..(83): X = S or Y
  MISC_FEATURE (90)..(90): X = Q or R
  MISC_FEATURE (103)..(103): X = Y or C
  MISC_FEATURE (105)..(105): X = I or V
  MISC_FEATURE (111)..(111): X = C or F
  MISC_FEATURE (118)..(118): X = H or Q
  MISC_FEATURE (151)..(151): X = L or V
  MISC_FEATURE (176)..(176): X = L or P
  MISC_FEATURE (181)..(181): X = E or K
  MISC_FEATURE (183)..(183): X = L or S
  MISC_FEATURE (184)..(184): X = I or T
  MISC_FEATURE (190)..(190): X = R or Q
  MISC_FEATURE (206)..(206): X = R or H
  MISC_FEATURE (266)..(266): X = Q or R
  MISC_FEATURE (277)..(277): X = H or L
  MISC_FEATURE (278)..(278): X = Q or P
  MISC_FEATURE (291)..(291): X = D or V
  MISC_FEATURE (299)..(299): X = A or V
  MISC_FEATURE (305)..(305): X = I or V
  MISC_FEATURE (322)..(322): X = A or T
  MISC_FEATURE (336)..(336): X = S or P
  MISC_FEATURE (347)..(347): X = G or A
  MISC_FEATURE (350)..(350): X = M or T
  MISC_FEATURE (354)..(354): X = L or F
  MISC_FEATURE (366)..(366): X = F or L
MEQDLRSIPASKLDKFIENHLPDTSFCADLREVIDXLCALLKDRXFXGPXRRMRASKGVKGKXTXLKGRSDADLVVFLNNLTXFEDQLNXQGVLIKEIKKQLXEXQHERRXGVKFEVXSLRSPNSRALSFKLSAPDLLKEVKFDVLPAYDXLDHLNILKKPNQQFYANLISGRTPXGKEGXLXXCFMGLXKYFLNCRPTKLKRLIXLVTHWYQLCKEKLGDPLPPQYALELLTVYAWEYGSRVTKFNTAQGFRTVLELVTKYKQLXIYWTVYYDFRXXEVSEYLHQQLKKXRPVILDPXDPTRNXAGLNPKDWRRLAGEAAXWLQYPCFKYRDGSXVCSWEVPTEVXVPXKYLXCRIFWLLFWSLXHFIFGKTSSG

SEQ ID NO: 4 - RNA, 1137 nt, Mus musculus
auggagcaagaucuuagauccauaccagcuuccaagcucgauaaguucauugagaaucaucugcccgacaccuccuuuugcgcugaccuuagagaggucauagaugccuugugcgcccuucucaaagaccgaucauuuagagguccuguccggcgcaugagagcaucaaagggggucaaggguaaagggaccacacuuaaggggaggucugacgccgaccuggucgucuuccucaacaacuugaccagcuuugaggaucagcucaaucaacaagguguacuuauaaaagagaucaaaaaacaacucugugaagugcagcacgaacgcaggugugguguuaaguuugaaguucauucacuucgcagucccaauucucgggcucucagcuuuaagcugucagcccccgacuugcucaaagaagugaaguuugaugugcugcccgcauacgaccuucucgaccaucucaacauacugaagaaaccaaaccaacaguuuuacgcaaaccucauuagcggacgaacuccaccuggcaaagaggggaaauugagcauauguuucaugggacuucggaaguacuuccugaacugccgccccacuaaacugaagaggcuuauuaggcucgucacucacugguaucaauuguguaaggagaagcugggggauccccucccaccucaauacgcacuugaacucuugaccguuuacgcaugggaguacggaagucgggucacuaaauucaauacagcucagggcuuucggaccguguuggaacucguaacuaaguauaagcaguugaggauauacuggacuguauauuaugauuuuagacaccaagagguuucagaguaccuucaucaacagcuuaaaaaagaucgacccguuauucuggacccugccgaccccacacgaaauaucgccggccuuaacccuaaggacuggagacgacuugcuggggaagcagccacuuggcuucaguaucccugcuucaaauauagagacggcagcccuguguguucuugggaaguccccacagaggucgggguccccaugaaauaucucuucugucgcaucuucuggcucuuguuuuggagucuguuccacuucaucuuugguaaaaccuccucagguuagugauaa

SEQ ID NO: 5 - RNA, 10 nt, Artificial Sequence
  Kozak sequence 5′UTR
  misc_feature (4)..(4): N = A or G
gccnccaugg

SEQ ID NO: 6 - RNA, 46 nt, Artificial Sequence
  synthetic sequence 5′UTR
  5′UTR (1)..(46)
ggaaauaagagagaaaagaagaguaagaagaaauauaagagccacc

SEQ ID NO: 7 - RNA, 47 nt, Artificial Sequence
  Synthetic sequence 5′UTR
  5′UTR (1)..(47)
gggaaauaagagagaaaagaagaguaagaagaaauauaagagccacc

SEQ ID NO: 8 - RNA, 47 nt, Artificial Sequence
  synthetic sequence 5′UTR
  5′UTR (1)..(47)
gggagaucagagagaaaagaagaguaagaagaaauauaagagccacc

SEQ ID NO: 9 - RNA, 145 nt, Artificial Sequence
  Synthetic sequence 5′UTR
  5′UTR (1)..(145)
ggaauaaaagucucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagcaac

SEQ ID NO: 10 - RNA, 42 nt, Artificial Sequence
  Synthetic sequence 5′UTR
  5′UTR (1)..(42)
gggagacaagcuuggcauuccgguacuguugguaaagccacc

SEQ ID NO: 11 - RNA, 92 nt, Artificial Sequence
  Synthetic sequence 5′UTR
  5′UTR (1)..(92)
ucaagcuuuuggacccucguacagaagcuaauacgacucacuauagggaaauaagagagaaaagaagaguaagaagaaauauaagagccacc

SEQ ID NO: 12 - RNA, 47 nt, Artificial Sequence
  Synthetic sequence 5′UTR
  5′UTR (1)..(47)
auggccggacccgcgacucagagcccaaugaagcugauggcucuuca

SEQ ID NO: 13 - RNA, 47 nt, Artificial Sequence; identical to SEQ ID NO: 7
  synthetic sequence 5′UTR
  5′UTR (1)..(47)

SEQ ID NO: 14 - RNA, 47 nt, Artificial Sequence
  Synthetic sequence 5′UTR
  5′UTR (1)..(47)
gggaauuaacagagaaaagaagaguaagaagaaauauaagagccacc

SEQ ID NO: 15 - RNA, 47 nt, Artificial Sequence
  Synthetic sequene 5′UTR
  5′UTR (1)..(47)
gggaaauuagacagaaaagaagaguaagaagaaauauaagagccacc

SEQ ID NO: 16 - RNA, 47 nt, Artificial Sequence
  Synthetic sequence 5′UTR
  5′UTR (1)..(47)
gggaaauaagagaguaaagaacaguaagaagaaauauaagagccacc

SEQ ID NO: 17 - RNA, 47 nt, Artificial Sequence
  Synthetic sequence 5′UTR
  5′UTR (1)..(47)
gggaaaaaagagagaaaagaagacuaagaagaaauauaagagccacc

SEQ ID NO: 18 - RNA, 47 nt, Artificial Sequence
  Synthetic sequence 5′UTR
  5′UTR (1)..(47)
gggaaauaagagagaaaagaagaguaagaagauauauaagagccacc

SEQ ID NO: 19 - RNA, 47 nt, Artificial Sequence
  Synthetic sequence 5′UTR
  5′UTR (1)..(47)
gggaaauaagagacaaaacaagaguaagaagaaauauaagagccacc

SEQ ID NO: 20 - RNA, 47 nt, Artificial Sequence
  Synthetic sequence 5′UTR
  5′UTR (1)..(47)
gggaaauuagagaguaaagaacaguaaguagaauuaaaagagccacc

SEQ ID NO: 21 - RNA, 47 nt, Artificial Sequence
  Synthetic sequence 5′UTR
  5′UTR (1)..(47)
gggaaauaagagagaauagaagaguaagaagaaauauaagagccacc

SEQ ID NO: 22 - RNA, 47 nt, Artificial Sequence
  Synthetic sequence 5′UTR
  5′UTR (1)..(47)
gggaaauaagagagaaaagaagaguaagaagaaaauuaagagccacc

SEQ ID NO: 23 - RNA, 47 nt, Artificial Sequence
  Synthetic sequence 5′UTR
  5′UTR (1)..(47)
gggaaauaagagagaaaagaagaguaagaagaaauuuaagagccacc

SEQ ID NO: 24 - RNA, 106 nt, Artificial Sequence
  Synthetic sequence 3′UTR
  3′UTR (1)..(106)
gcugccuucugcggggcuugccuucuggccaugcccuucuucucucccuugcaccuguaccucuuggucuuugaauaaagccugaguaggaaggcggccgcaaaaa

SEQ ID NO: 25 - RNA, 371 nt, Artificial Sequence
  Synthetic sequence 3′UTR
  3′UTR (1)..(371)
gcgccugcccaccugccaccgacugcuggaacccagccagugggagggccuggcccaccagaguccugcucccucacuccucgccccgcccccugucccagagucccaccugggggcucucuccacccuucucagaguuccaguuucaaccagaguuccaaccaaugggcuccauccucuggauucuggccaaugaaauaucucccuggcaggguccucuucuuuucccagagcuccaccccaaccaggagcucuaguuaauggagagcucccagcacacucggagcuugugcuuugucuccacgcaaagcgauaaauaaaagcauugguggccuuuggucuuugaauaaagccugaguaggaagucuaga

SEQ ID NO: 26 - RNA, 568 nt, Homo sapiens
  3′UTR (1)..(567)
gccccugccgcucccacccccacccaucugggccccggguucaagagagagcggggucugaucucguguagccauauagaguuugcuucugagugucugcuuuguuuaguagaggugggcaggaggagcugaggggcuggggcugggguguugaaguuggcuuugcaugcccagcgaugcgccucccugugggaugucaucacccugggaaccgggaguggcccuuggcucacuguguucugcaugguuuggaucugaauuaauuguccuuucuucuaaaucccaaccgaacuucuuccaaccuccaaacuggcuguaaccccaaauccaagccauuaacuacaccugacaguagcaauugucugauuaaucacuggccccuugaagacagcagaaugucccuuugcaaugaggaggagaucugggcugggcgggccagcuggggaagcauuugacuaucuggaacuugugugugccuccucagguauggcagugacucaccugguuuuaauaaaacaaccugcaacaucucauggucuuugaauaaagccugaguaggaagucuaga

SEQ ID NO: 27 - RNA, 289 nt, Homo sapiens
  3′UTR (1)..(289)
acacacuccaccuccagcacgcgacuucucaggacgacgaaucuucucaaugggggggcggcugagcuccagccaccccgcagucacuuucuuuguaacaacuuccguugcugccaucguaaacugacacaguguuuauaacguguacauacauuaacuuauuaccucauuuuguuauuuuucgaaacaaagcccuguggaagaaaauggaaaacuugaagaagcauuaaagucauucuguuaagcugcguaaauggucuuugaauaaagccugaguaggaagucuaga

SEQ ID NO: 28 - RNA, 379 nt, Homo sapiens
  3′UTR (1)..(379)
caucacauuuaaaagcaucucagccuaccaugagaauaagagaaagaaaaugaagaucaaaagcuuauucaucuguuuuucuuuuucguugguguaaagccaacacccugucuaaaaaacauaaauuucuuuaaucauuuugccucuuuucucugugcuucaauuaauaaaaaauggaaagaaucuaauagagugguacagcacuguuauuuuucaaagauguguugcuauccugaaaauucuguagguucuguggaaguuccaguguucucucuuauuccacuucgguagaggauuucuaguuucuugugggcuaauuaaauaaaucauuaauacucuucuaauggucuuugaauaaagccugaguaggaagucuaga

SEQ ID NO: 29 - RNA, 118 nt, Artificial Sequence
  Synthetic sequence 3′UTR
  3′UTR (1)..(118)
gcugccuucugcggggcuugccuucuggccaugcccuucuucucucccuugcaccuguaccucuuggucuuugaauaaagccugaguaggaaggcggccgcucgagcaugcaucuaga

SEQ ID NO: 30 - RNA, 908 nt, Homo sapiens
  3′UTR (1)..(908)
gccaagcccuccccaucccauguauuuaucucuauuuaauauuuaugucuauuuaagccucauauuuaaagacagggaagagcagaacggagccccaggccucuguguccuucccugcauuucugaguuucauucuccugccuguagcagugagaaaaagcuccuguccucccauccccuggacugggagguagauagguaaauaccaaguauuuauuacuaugacugcuccccagcccuggcucugcaaugggcacugggaugagccgcugugagccccugguccugaggguccccaccugggacccuugagaguaucaggucucccacgugggagacaagaaaucccuguuuaauauuuaaacagcaguguuccccaucuggguccuugcaccccucacucuggccucagccgacugcacagcggccccugcauccccuuggcugugaggccccuggacaagcagagguggccagagcugggaggcauggcccuggggucccacgaauuugcuggggaaucucguuuuucuucuuaagacuuuugggacaugguuugacucccgaacaucaccgacgcgucuccuguuuuucuggguggccucgggacaccugcccugcccccacgagggucaggacugugacucuuuuuagggccaggcaggugccuggacauuugccuugcuggacggggacuggggaugugggagggagcagacaggaggaaucaugucaggccugugugugaaaggaagcuccacugucacccuccaccucuucaccccccacucaccaguguccccuccacugucacauuguaacugaacuucaggauaauaaaguguuugccuccauggucuuugaauaaagccugaguaggaaggcggccgcucgagcaugcaucuaga

SEQ ID NO: 31 - RNA, 835 nt, Homo sapiens
  3′UTR (1)..(835)
acucaaucuaaauuaaaaaagaaagaaauuugaaaaaacuuucucuuugccauuucuucuucuucuuuuuuaacugaaagcugaauccuuccauuucuucugcacaucuacuugcuuaaauugugggcaaaagagaaaaagaaggauugaucagagcauugugcaauacaguuucauuaacuccuucccccgcucccccaaaaauuugaauuuuuuuuucaacacucuuacaccuguuauggaaaaugucaaccuuuguaagaaaaccaaaauaaaaauugaaaaauaaaaaccauaaacauuugcaccacuuguggcuuuugaauaucuuccacagagggaaguuuaaaacccaaacuuccaaagguuuaaacuaccucaaaacacuuucccaugagugugauccacauuguuaggugcugaccuagacagagaugaacugagguccuuguuuuguuuuguucauaauacaaaggugcuaauuaauaguauuucagauacuugaagaauguugauggugcuagaagaauuugagaagaaauacuccuguauugaguuguaucgugugguguauuuuuuaaaaaauuugauuuagcauucauauuuuccaucuuauucccaauuaaaaguaugcagauuauuugcccaaaucuucuucagauucagcauuuguucuuugccagucucauuuucaucuucuuccaugguuccacagaagcuuuguuucuugggcaagcagaaaaauuaaauuguaccuauuuuguauaugugagauguuuaaauaaauugugaaaaaaaugaaauaaagcauguuugguuuuccaaaagaacauau

SEQ ID NO: 32 - RNA, 297 nt, Homo sapiens
  3′UTR (1)..(297)
cgccgccgcccgggccccgcagucgagggucgugagcccaccccguccauggugcuaagcgggcccgggucccacacggccagcaccgcugcucacucggacgacgcccugggccugcaccucuccagcuccucccacgggguccccguagccccggcccccgcccagccccaggucuccccaggcccuccgcaggcugcccggccucccucccccugcagccaucccaaggcuccugaccuaccuggccccugagcucuggagcaagcccugacccaauaaaggcuuugaacccau

SEQ ID NO: 33 - RNA, 602 nt, Homo sapiens
  3′UTR (1)..(602)
ggggcuagagcccucuccgcacagcguggagacggggcaaggagggggguuauuaggauuggugguuuuguuuugcuuuguuuaaagccgugggaaaauggcacaacuuuaccucugugggagaugcaacacugagagccaaggggugggaguugggauaauuuuuauauaaaagaaguuuuuccacuuugaauugcuaaaaguggcauuuuuccuaugugcagucacuccucucauuucuaaaauagggacguggccaggcacgguggcucaugccuguaaucccagcacuuugggaggccgaggcaggcggcucacgaggucaggagaucgagacuauccuggcuaacacgguaaaacccugucucuacuaaaaguacaaaaaauuagcugggcgugguggugggcaccuguagucccagcuacucgggaggcugaggcaggagaaaggcaugaauccaagaggcagagcuugcagugagcugagaucacgccauugcacuccagccugggcaacaguguuaagacucugucucaaauauaaauaaauaaauaaauaaauaaauaaauaaauaaaaauaaagcgagauguugcccucaaa

SEQ ID NO: 34 - RNA, 119 nt, Artificial Sequence
  Synthetic Polynucleotide
  3′UTR (1)..(119)
ugauaauaggcuggagccucgguggccaugcuucuugccccuugggccuccccccagccccuccuccccuuccugcacccguacccccguggucuuugaauaaagucugagugggcggc

SEQ ID NO: 35 - RNA, 3001 nt, Artificial Sequence
  Synthetic sequence 3′UTR
  3′UTR (1)..(3001)
auauuaaggaucaagcuguuagcuaauaaugccaccucugcaguuuugggaacaggcaaauaaaguaucaguauacauggugauguacaucuguagcaaagcucuuggagaaaaugaagacugaagaaagcaaagcaaaaacuguauagagagauuuuucaaaagcaguaaucccucaauuuuaaaaaaggauugaaaauucuaaaugucuuucugugcauauuuuuuguguuaggaaucaaaaguauuuuauaaaaggagaaagaacagccucauuuuagauguaguccuguuggauuuuuuaugccuccucaguaaccagaaauguuuuaaaaaacuaaguguuuaggauuucaagacaacauuauacauggcucugaaauaucugacacaauguaaacauugcaggcaccugcauuuuauguuuuuuuuuucaacaaaugugacuaauuugaaacuuuuaugaacuucugagcuguccccuugcaauucaaccgcaguuugaauuaaucauaucaaaucaguuuuaauuuuuuaaauuguacuucagagucuauauuucaagggcacauuuucucacuacuauuuuaauacauuaaaggacuaaauaaucuuucagagaugcuggaaacaaaucauuugcuuuauauguuucauuagaauaccaaugaaacauacaacuugaaaauuaguaauaguauuuuugaagaucccauuucuaauuggagaucucuuuaauuucgaucaacuuauaauguguaguacuauauuaagugcacuugaguggaauucaacauuugacuaauaaaaugaguucaucauguuggcaagugauguggcaauuaucucuggugacaaaagaguaaaaucaaauauuucugccuguuacaaauaucaaggaagaccugcuacuaugaaauagaugacauuaaucugucuucacuguuuauaauacggauggauuuuuuuucaaaucaguguguguuuugaggucuuauguaauugaugacauuugagagaaaugguggcuuuuuuuagcuaccucuuuguucauuuaagcaccaguaaagaucaugucuuuuuauagaaguguagauuuucuuugugacuuugcuaucgugccuaaagcucuaaauauaggugaaugugugaugaauacucagauuauuugucucucuauauaauuaguuugguacuaaguuucucaaaaaauuauuaacacaugaaagacaaucucuaaaccagaaaaagaaguaguacaaauuuuguuacuguaaugcucgcguuuagugaguuuaaaacacacaguaucuuuugguuuuauaaucaguuucuauuuugcugugccugagauuaagaucuguguaugugugugugugugugugugcguuuguguguuaaagcagaaaagacuuuuuuaaaaguuuuaagugauaaaugcaauuuguuaauugaucuuagaucacuaguaaacucagggcugaauuauaccauguauauucuauuagaagaaaguaaacaccaucuuuauuccugcccuuuuucuucucucaaaguaguuguaguuauaucuagaaagaagcaauuuugauuucuugaaaagguaguuccugcacucaguuuaaacuaaaaauaaucauacuuggauuuuauuuauuuuugucauaguaaaaauuuuaauuuauauauauuuuuauuuaguauuaucuuauucuuugcuauuugccaauccuuugucaucaauuguguuaaaugaauugaaaauucaugcccuguucauuuuauuuuacuuuauugguuaggauauuuaaaggauuuuuguauauauaauuucuuaaauuaauauuccaaaagguuaguggacuuagauuauaaauuauggcaaaaaucuaaaaacaacaaaaaugauuuuuauacauucuauuucauuauuccucuuuuuccaauaagucauacaauugguagauaugacuuauuuuauuuuuguauuauucacuauaucuuuaugauauuuaaguauaaauaauuaaaaaaauuuauuguaccuuauagucugucaccaaaaaaaaaaaauuaucuguagguagugaaaugcuaauguugauuugucuuuaagggcuuguuaacuauccuuuauuuucucauuugucuuaaauuaggaguuuguguuuaaauuacucaucuaagcaaaaaauguauauaaaucccauuacuggguauauacccaaaggauuauaaaucaugcugcuauaaagacacaugcacacguauguuuauugcagcacuauucacaauagcaaagacuuggaaccaacccaaauguccaucaaugauagacuugauuaagaaaaugugcacauauacaccauggaauacuaugcagccauaaaaaaggaugaguucauguccuuuguagggacauggauaaagcuggaaaccaucauucugagcaaacuauugcaaggacagaaaaccaaacacugcauguucucacucauaggugggaauugaacaaugagaacacuuggacacaagguggggaacaccacacaccagggccugucaugggguggggggaguggggagggauagcauuaggagauauaccuaauguaaaugaugaguuaaugggugcagcacaccaacauggcacauguauacauauguagcaaaccugcacguugugcacauguacccuagaacuuaaaguauaauuaaaaaaaaaaagaaaacagaagcuauuuauaaagaaguuauuugcugaaauaaaugugaucuuucccauuaaaaaaauaaagaaauuuugggguaaaaaaacacaauauauuguauucuugaaaaauucuaagagaguggaugugaaguguucucaccacaaaagugauaacuaauugagguaaugcacauauuaauuagaaagauuuugucauuccacaauguauauauacuuaaaaauauguuauacacaauaaauacauacauuaaaaaauaaguaaaugua

SEQ ID NO: 36 - RNA, 1037 nt, Homo sapiens
  3′UTR (1)..(1037)
cccacccugcacgccggcaccaaacccuguccucccaccccuccccacucaucacuaaacagaguaaaaugugaugcgaauuuucccgaccaaccugauucgcuagauuuuuuuuaaggaaaagcuuggaaagccaggacacaacgcugcugccugcuuugugcaggguccuccggggcucagcccugaguuggcaucaccugcgcagggcccucuggggcucagcccugagcuagugucaccugcacagggcccucugaggcucagcccugagcuggcgucaccugugcagggcccucuggggcucagcccugagcuggccucaccuggguuccccaccccgggcucuccugcccugcccuccugcccgcccucccuccugccugcgcagcuccuucccuaggcaccucugugcugcaucccaccagccugagcaagacgcccucucggggccugugccgcacuagccucccucuccucuguccccauagcugguuuuucccaccaauccucaccuaacaguuacuuuacaauuaaacucaaagcaagcucuucuccucagcuuggggcagccauuggccucugucucguuuugggaaaccaaggucaggaggccguugcagacauaaaucucggcgacucggccccgucuccugaggguccugcuggugaccggccuggaccuuggcccuacagcccuggaggccgcugcugaccagcacugaccccgaccucagagaguacucgcaggggcgcuggcugcacucaagacccucgagauuaacggugcuaaccccgucugcuccucccucccgcagagacuggggccuggacuggacaugagagccccuuggugccacagagggcugugucuuacuagaaacaacgcaaaccucuccuuccucagaauagugauguguucgacguuuuaucaaaggcccccuuucuauguucauguuaguuuugcuccuucuguguuuuuuucugaaccauauccauguugcugacuuuuccaaauaaagguuuucacuccucuc

SEQ ID NO: 37 - RNA, 577 nt, Homo sapiens
  3′UTR (1)..(577)
agaggccugccuccagggcuggacugaggccugagcgcuccugccgcagagcuggccgcgccaaauaaugucucugugagacucgagaacuuucauuuuuuuccaggcugguucggauuugggguggauuuugguuuuguuccccuccuccacucucccccacccccuccccgcccuuuuuuuuuuuuuuuuuuaaacugguauuuuaucuuugauucuccuucagcccucaccccugguucucaucuuucuugaucaacaucuuuucuugccucuguccccuucucucaucucuuagcuccccuccaaccuggggggcagugguguggagaagccacaggccugagauuucaucugcucuccuuccuggagcccagaggagggcagcagaaggggguggugucuccaaccccccagcacugaggaagaacggggcucuucucauuucaccccucccuuucuccccugcccccaggacugggccacuucuggguggggcagugggucccagauuggcucacacugagaauguaagaacuacaaacaaaauuucuauuaaauuaaauuuugugucucc

SEQ ID NO: 38 - RNA, 2212 nt, Homo sapiens
  3′UTR (1)..(2212)
cucccuccaucccaaccuggcucccucccacccaaccaacuuuccccccaacccggaaacagacaagcaacccaaacugaacccccucaaaagccaaaaaaugggagacaauuucacauggacuuuggaaaauauuuuuuuccuuugcauucaucucucaaacuuaguuuuuaucuuugaccaaccgaacaugaccaaaaaccaaaagugcauucaaccuuaccaaaaaaaaaaaaaaaaaaagaauaaauaaauaacuuuuuaaaaaaggaagcuugguccacuugcuugaagacccaugcggggguaagucccuuucugcccguugggcuuaugaaaccccaaugcugcccuuucugcuccuuucuccacaccccccuuggggccuccccuccacuccuucccaaaucugucuccccagaagacacaggaaacaauguauugucugcccagcaaucaaaggcaaugcucaaacacccaaguggcccccacccucagcccgcuccugcccgcccagcacccccaggcccugggggaccugggguucucagacugccaaagaagccuugccaucuggcgcucccauggcucuugcaacaucuccccuucguuuuugagggggucaugccgggggagccaccagccccucacuggguucggaggagagucaggaagggccacgacaaagcagaaacaucggauuuggggaacgcgugucaaucccuugugccgcagggcugggcgggagagacuguucuguuccuuguguaacuguguugcugaaagacuaccucguucuugucuugaugugucaccggggcaacugccugggggcggggaugggggcaggguggaagcggcuccccauuuuauaccaaaggugcuacaucuaugugaugggugggguggggagggaaucacuggugcuauagaaauugagaugcccccccaggccagcaaauguuccuuuuuguucaaagucuauuuuuauuccuugauauuuuucuuuuuuuuuuuuuuuuuuuguggauggggacuugugaauuuuucuaaaggugcuauuuaacaugggaggagagcgugugcggcuccagcccagcccgcugcucacuuuccacccucucuccaccugccucuggcuucucaggccucugcucuccgaccucucuccucugaaacccuccuccacagcugcagcccauccucccggcucccuccuagucuguccugcguccucuguccccggguuucagagacaacuucccaaagcacaaagcaguuuuucccccuaggggugggaggaagcaaaagacucuguaccuauuuuguauguguauaauaauuugagauguuuuuaauuauuuugauugcuggaauaaagcauguggaaaugacccaaacauaauccgcaguggccuccuaauuuccuucuuuggaguugggggagggguagacauggggaaggggcuuuggggugaugggcuugccuuccauuccugcccuuucccuccccacuauucucuucuagaucccuccauaaccccacuccccuuucucucacccuucuuauaccgcaaaccuuucuacuuccucuuucauuuucuauucuugcaauuuccuugcaccuuuuccaaauccucuucuccccugcaauaccauacaggcaauccacgugcacaacacacacacacacucuucacaucugggguuguccaaaccucauacccacuccccuucaagcccauccacucuccacccccuggaugcccugcacuugguggcggugggaugcucauggauacugggagggugaggggaguggaacccgugaggaggaccugggggccucuccuugaacugacaugaagggucaucuggccucugcucccuucucacccacgcugaccuccugccgaaggagcaacgcaacaggagaggggucugcugagccuggcgagggucugggagggaccaggaggaaggcgugcucccugcucgcuguccuggcccugggggagugagggagacagacaccugggagagcuguggggaaggcacucgcaccgugcucuugggaaggaaggagaccuggcccugcucaccacggacugggugccucgaccuccugaauccccagaacacaaccccccugggcugggguggucuggggaaccaucgugcccccgccucccgccuacuccuuuuuaagcuu

SEQ ID NO: 39 - RNA, 729 nt, Homo sapiens
  3′UTR (1)..(729)
uuggccaggccugacccucuuggaccuuucuucuuugccgacaaccacugcccagcagccucugggaccucggggucccagggaacccaguccagccuccuggcuguugacuucccauugcucuuggagccaccaaucaaagagauucaaagagauuccugcaggccagaggcggaacacaccuuuauggcuggggcucuccgugguguucuggacccagccccuggagacaccauucacuuuuacugcuuuguagugacucgugcucuccaaccugucuuccugaaaaaccaaggcccccuucccccaccucuuccauggggugagacuugagcagaacaggggcuuccccaaguugcccagaaagacugucugggugagaagccauggccagagcuucucccaggcacagguguugcaccagggacuucugcuucaaguuuugggguaaagacaccuggaucagacuccaagggcugcccugagucugggacuucugccuccauggcuggucaugagagcaaaccguaguccccuggagacagcgacuccagagaaccucuugggagacagaagaggcaucugugcacagcucgaucuucuacuugccuguggggaggggagugacagguccacacaccacacugggucacccuguccuggaugccucugaagagagggacagaccgucagaaacuggagaguuucuauuaaaggucauuuaaacca

SEQ ID NO: 40 - RNA, 847 nt, Homo sapiens
  3′UTR (1)..(847)
uccuccgggaccccagcccucaggauuccugaugcuccaaggcgacugaugggcgcuggaugaaguggcacagucagcuucccugggggcuggugucauguugggcuccuggggcgggggcacggccuggcauuucacgcauugcugccaccccagguccaccugucuccacuuucacagccuccaagucuguggcucuucccuucuguccuccgaggggcuugccuucucucguguccagugaggugcucagugaucggcuuaacuuagagaagcccgcccccuccccuucuccgucugucccaagagggucugcucugagccugcguuccuagguggcucggccucagcugccuggguuguggccgcccuagcauccuguaugcccacagcuacuggaauccccgcugcugcuccgggccaagcuucugguugauuaaugagggcaugggguggucccucaagaccuuccccuaccuuuuguggaaccagugaugccucaaagacaguguccccuccacagcugggugccaggggcaggggauccucaguauagccggugaacccugauaccaggagccugggccucccugaaccccuggcuuccagccaucucaucgccagccuccuccuggaccucuuggcccccagccccuuccccacacagccccagaagggucccagagcugaccccacuccaggaccuaggcccagccccucagccucaucuggagccccugaagaccagucccacccaccuuucuggccucaucugacacugcuccgcauccugcuguguguccuguuccauguuccgguuccauccaaauacacuuucuggaacaaa

SEQ ID NO: 41 - RNA, 110 nt, Artificial Sequence
  Synthetic sequence 3′UTR
  3′UTR (1)..(110)
gcuggagccucgguggccaugcuucuugccccuugggccuccccccagccccuccuccccuuccugcacccguacccccguggucuuugaauaaagucugagugggcggc

SEQ ID NO: 42 - RNA, 1289 nt, Mus musculus
ggaaauaagagagaaaagaagaguaagaagaaauauaagagccaccauggagcaagaucuuagauccauaccagcuuccaagcucgauaaguucauugagaaucaucugcccgacaccuccuuuugcgcugaccuuagagaggucauagaugccuugugcgcccuucucaaagaccgaucauuuagagguccuguccggcgcaugagagcaucaaagggggucaaggguaaagggaccacacuuaaggggaggucugacgccgaccuggucgucuuccucaacaacuugaccagcuuugaggaucagcucaaucaacaagguguacuuauaaaagagaucaaaaaacaacucugugaagugcagcacgaacgcaggugugguguuaaguuugaaguucauucacuucgcagucccaauucucgggcucucagcuuuaagcugucagcccccgacuugcucaaagaagugaaguuugaugugcugcccgcauacgaccuucucgaccaucucaacauacugaagaaaccaaaccaacaguuuuacgcaaaccucauuagcggacgaacuccaccuggcaaagaggggaaauugagcauauguuucaugggacuucggaaguacuuccugaacugccgccccacuaaacugaagaggcuuauuaggcucgucacucacugguaucaauuguguaaggagaagcugggggauccccucccaccucaauacgcacuugaacucuugaccguuuacgcaugggaguacggaagucgggucacuaaauucaauacagcucagggcuuucggaccguguuggaacucguaacuaaguauaagcaguugaggauauacuggacuguauauuaugauuuuagacaccaagagguuucagaguaccuucaucaacagcuuaaaaaagaucgacccguuauucuggacccugccgaccccacacgaaauaucgccggccuuaacccuaaggacuggagacgacuugcuggggaagcagccacuuggcuucaguaucccugcuucaaauauagagacggcagcccuguguguucuugggaaguccccacagaggucgggguccccaugaaauaucucuucugucgcaucuucuggcucuuguuuuggagucuguuccacuucaucuuugguaaaaccuccucagguuagugauaagcugccuucugcggggcuugccuucuggccaugcccuucuucucucccuugcaccuguaccucuuggucuuugaauaaagccugaguaggaaggcggccgcaaaaa

(19) United States
(12) Patent Application Publication
(10) Pub. No.: US 2021/0315980 A1
(43) Pub. Date: Oct. 14, 2021

(54) TREATMENT OF FLAVIVIRUS INFECTIONS IN HUMANS USING MUS MUSCULUS RESISTANT 2'-5' OLIGOADENYLATE SYNTHETASE 1B

(71) Applicants: Georgia State University Research Foundation, Inc., Atlanta, GA (US); Georgia Tech Research Corporation, Atlanta, GA (US)

(72) Inventors: Margo A. Brinton, Atlanta, GA (US); Joseph C. Madden, Jr., Atlanta, GA (US); Philip J. Santangelo, Atlanta, GA (US)

(21) Appl. No.: 17/269,968

(22) PCT Filed: Aug. 21, 2019
(86) PCT No.: PCT/US2019/047548

§ 371 Date: Feb. 19, 2021

Related U.S. Application Data
(60) Provisional application No. 62/720,783, filed on Aug. 21, 2018.

(51) Int. Cl. A61K 38/45; A61K 47/24; A61K 47/18; A61K 48/00; A61P 31/14

(52) U.S. Cl. CPC A61K 38/45; A61K 47/24; A61P 31/14; A61K 48/0066; A61K 47/18

(57) ABSTRACT

The present invention provides a method for treating, and compositions useful for treating, Flavivirus infections in a human by administering to the human an effective amount of mRNA encoding Mus musculus resistant 2′-5′ oligoadenylate synthetase 1b (rOas1b), or variants thereof.

### STATEMENT OF RELATED APPLICATIONS

This application claims the benefit of U.S. Provisional Application No. 62/720,783, filed on Aug. 21, 2018. The entirety of this application is incorporated herein by reference for all purposes.

### STATEMENT OF GOVERNMENT SUPPORT

The U.S. Government has rights in this invention by virtue of support under Grant No. R01 AI45135 awarded by the National Institutes of Allergy and Infectious Disease.

### FIELD OF THE INVENTION

The invention is directed to the use of mRNA encoding Mus musculus resistant 2′-5′ oligoadenylate synthetase 1b for the treatment in humans of medical disorders caused by members of the genus Flavivirus.

### INCORPORATION BY REFERENCE

The contents of the text file named “17041-006WO1SequenceListing_ST25.txt,” which was created on Aug. 21, 2019 and is 40 KB in size, are hereby incorporated by reference in their entirety.

### BACKGROUND OF THE INVENTION

The Flaviviridae family is composed of four genera: Flavivirus, Hepacivirus, Pegivirus, and Pestivirus. Flaviviridae viruses have single-stranded ribonucleic acid (ssRNA) genomes and replicate following the positive stranded RNA virus replication model. Replication of the positive-sense ssRNA genome progresses through double-stranded RNA (dsRNA) intermediates and typically occurs within invaginations of endoplasmic reticular membranes. Infections with viruses of the genus Flavivirus, such as West Nile, Japanese encephalitis, tick-borne encephalitis, yellow fever, Zika and dengue virus, induce a wide-range of conditions in humans ranging from asymptomatic or mild flu-like symptoms to meningitis, encephalitis, or paralysis, which can be fatal. Recently, Zika virus infections during pregnancy have been linked to miscarriage and can cause microcephaly, a potentially fatal congenital brain condition. To date, few vaccines and no antiviral therapies have been successfully developed for the treatment of Flavivirus infections.

The 2′-5′ oligoadenylate synthetase (OAS)/Ribonuclease (RNase) L pathway functions as an innate host defense response against viral infections. OAS gene expression is upregulated by the signaling of interferons produced by cells in response to a viral infection (SN Sarkar and GC Sen, “Novel functions of proteins encoded by viral stress-inducible genes,” (2004) Pharmacol. Ther. 103:245-259). Viral double-stranded RNA (dsRNA) binds to and activates OAS, causing it to polymerize ATP into short 2′-5′-linked oligomers (2-5A) (Justesen et al., “Gene structure and function of the 2′-5′-oligoadenylate synthetase family,” (2000) Cell. Mol. Life Sci. 57:1593-1612). These 2-5A oligomers bind to and activate latent endoribonuclease L (RNase L,) which is constitutively expressed in cells. The activated RNase L subsequently cleaves viral and cellular single-stranded RNAs (Courtney et al., “Identification of novel host cell binding partners of Oas1b, the protein conferring resistance to Flavivirus-induced disease in mice,” (2012) J. Virol. 86(15); 7953-7963).

A dominant allele of the Fly gene discovered in the 1920s in mice reduces the replication efficiency of members of the genus Flavivirus and confers resistance to Flavivirus-induced disease. Mice carrying the dominant resistant allele are still susceptible to infection but produce significantly lower levels of virus compared to mice which are homozygous for the susceptibility allele. The Fly gene was later found to encode oligoadenylate synthetase 1b (Oas1b), with resistant mice expressing a full-length protein while susceptible mice express a truncated version due to a premature stop codon (Perelygin et al. “Positional cloning of the murine flavivirus resistance gene” PNAS 2002, 99(14):9322-9327). The Oas/RNase L pathway is typically virus-nonspecific while the Fly gene shows a Flavivirus-specific phenotype, suggesting that Oas1b mediates Flavivirus-resistance through a different mechanism than the Oas/RNase L pathway. Indeed, Oas1b was found to have no 2-5A synthetase activity and in fact suppresses 2-5A synthesis within intact cells (Elbanesh et al. “The Flv^{r}-encoded murine oligoadenylate synthetase 1b (Oas1b) suppresses 2-5A synthesis in intact cells” Virology 2011, 409(2): 262-270). Oas1b is the only member of the murine Oas family that has a C-terminal transmembrane domain, which targets it to the endoplasmic reticular membrane. It has been found to bind to ATP binding cassette protein 3, subfamily F (ABCF3). Knockdown of ABCF3 has been shown to increase the replication of West Nile virus, suggesting that ABCF3 is a component in the Oas1b-mediated resistance mechanism (Courtney et al. “Identification of Novel Host Cell Binding Partners of Oas1b, the Protein Conferring Resistance to Flavivirus-Induced Disease in Mice” Journal of Virology 2012, 86(15):7953-7963). While the Flv^{r} allele significantly inhibits the proliferation of Flavivirus in mice, no confirmed parallel appears to exist within other mammalian species, including humans.

Mohapatra et al. describes the treatment of an RNA virus infection in a patient by administering a nucleotide sequence encoding an enzymatically active 2′-5′ oligoadenylate synthetase protein in U.S. Pat. No. 8,293,717 titled “Materials and Methods for Prevention and Treatment of Viral Diseases.”

Brighton et al. describes a method of identifying genes for Flavivirus resistance in International Patent Publication No. WO2004000998 title “Compositions and Methods for Viral Resistance Genes.”

Due to the increasing incidence of Flavivirus infections around the world, there is a clear need for new therapies to treat these infections.

### SUMMARY OF THE INVENTION

The present invention provides a method for treating, and compositions useful for treating, Flavivirus infections in a human by administering to the human an effective amount of mRNA encoding Mus musculus resistant 2′-5′ oligoadenylate synthetase 1b (rOas1b), or variants thereof. It has been discovered that introducing mRNA encoding mouse resistant rOas1b to human cells reduces Flavivirus RNA levels without activation of the innate human Oas/RNase L pathway, provides an efficacious antiviral effect against all members of the genus Flavivirus, and is effective against already established infections, inhibiting both viral RNA replication and allowing the infected cell to subsequently clear the viral material. Advantageously, the use of mRNA leads to the transient expression of rOas1b upon delivery, avoiding potential complications from long-term expression seen with DNA construct and viral vector delivery. It is surprising that such results are observed upon the expression of rOas1b in interspecies human cells upon transcription of the delivered mRNA without any associated cellular toxicity or long term cellular impairment, as no protein with similar anti-Flavivirus activity has been identified in humans. The methods and compositions described herein can be used to treat any Flavivirus infection in a human, for example but not limited to, West Nile virus, yellow fever virus, tick-borne encephalitis virus, Dengue virus, Japanese encephalitis virus, or Zika virus.

The mRNA for administration includes a coding region encoding murine resistant 2′-5′ oligoadenylate synthetase 1b (rOas1b) or a variant thereof that is 80%, 85%, 90%, 95%, 96%, 97%, 98%, 99% or more homologous. In one embodiment, the mRNA comprises a coding region encoding the polypeptide of SEQ. ID. NO.: 1 (UniProt KB—Q60856) or a variant thereof that is 80%, 85%, 90%, 95%, 96%, 97%, 98%, 99% or more homologous.

In certain embodiments, the mRNA for administration includes a coding region encoding a polypeptide of SEQ. ID. NO.: 1, wherein the polypeptide includes one or more amino acid substitution selected from an A36S substitution, S45F substitution, R47Q substitution, V50G substitution, G63C substitution, T65A substitution, S83Y substitution, Q90R substitution, C103Y substitution, V105I substitution, C111F substitution, H118Q substitution, L151V substitution, P176L substitution, K181E substitution, S183L substitution, I184T substitution, R190Q substitution, R206H substitution, Q266R substitution, H277L substitution, Q278P substitution, D291V substitution, A299V substitution, I305V substitution, A322T substitution, S336P substitution, G347A substitution, M350T substitution, L354F substitution, or F368L substitution, or any combination thereof, or a variant thereof that is 80%, 85%, 90%, 95%, 96%, 97%, 98%, 99% or more homologous.

In one embodiment, the mRNA for administration includes a coding region encoding a polypeptide of SEQ. ID. NO.: 2 or a variant thereof that is 80%, 85%, 90%, 95%, 96%, 97%, 98%, 99% or more homologous. In another embodiment, the mRNA for administration comprises a coding region encoding a polypeptide of SEQ. ID. NO.: 3 or a variant thereof that is 80%, 85%, 90%, 95%, 96%, 97%, 98%, 99% or more homologous. In still another embodiment, the mRNA for administration includes a coding region comprising SEQ. ID. NO.: 4, or a variant thereof that is 80%, 85%, 90%, 95%, 96%, 97%, 98%, 99% or more homologous.

The mRNA for administration can be codon optimized. By codon optimizing, the formation of secondary structures can be reduced and translational efficiency improved. In certain embodiments, the codon optimization includes GC enrichment of the coding region. In certain embodiments, the codon optimization includes codon quality enrichment of the coding region. Suitable codon optimization for incorporation into the mRNA of the present invention are described further below.

In certain aspects, the mRNA for administration includes a 5′ untranslated region (5′UTR) operably linked to the 5′ end of the coding region encoding rOAS1b, and a 3′ untranslated region (3′UTR) operably linked to the 3′ end of the coding region encoding rOAS1b. The 5′ UTR and 3′ UTR can be selected from any suitable 5′ UTR and 3′ UTR, for example a synthetic 5′ UTR and/or 3′ UTR, a naturally occurring or naturally derived 5′ UTR and/or 3′ UTR, or a combination thereof. In certain embodiments, the 5′ UTR and/or 3′ UTR is derived from a human 5′ UTR and/or 3′ UTR. The use of human-derived UTRs may facilitate the expression of rOAS1b in human cells. In certain embodiments, the 5′ UTR and/or 3′ UTR are derived from a 5′ UTR and/or 3′ UTR naturally expressed in the targeted tissue for treatments, i.e., an untranslated region from an mRNA expressed in, for example the liver, brain, or testes. Additional suitable 5′ UTR and 3′ UTRs are further described below.

In one embodiment, the 5′ UTR operable linked to the rOAS1b coding region comprises GGAAAUAAGAGAGAAAAGAAGAGUAAGAAGAAAUAUAAGAGCCACC (SEQ. ID. NO.: 6), or a variant thereof that is 80%, 85%, 90%, 95%, 96%, 97%, 98%, 99% or more homologous.

In one embodiment, the 3′ UTR operably linked to the rOAS1b coding region comprises GCUGCCUUCUGCGGGGCUUGCCUUCUGGCCAUGCCCUUCUUCUCUCCCUUGCACC UGUACCUCUUGGUCUUUGAAUAAAGCCUGAGUAGGAAGGCGGCCGCAAAAA (SEQ. ID. NO.: 24), or a variant thereof that is 80%, 85%, 90%, 95%, 96%, 97%, 98%, 99% or more homologous.

In one embodiment, the mRNA for administration includes a 5′ UTR, a coding region, and a 3′ UTR comprising SEQ. ID. NO.: 42 or a variant thereof that is 80%, 85%, 90%, 95%, 96%, 97%, 98%, 99% or more homologous.

The mRNA for administration may further include a 5′ terminal cap operably linked to the 5′ end of the mRNA. The 5′ terminal cap may include a naturally occurring cap, a synthetic cap, or an optimized cap as further described herein. In addition, the mRNA for administration may further include a 3′ tailing sequence, as further described herein. The 3′ tailing sequence may include a naturally occurring tailing sequence or a synthetic tailing sequence and/or a chain terminating nucleoside. Non-limiting examples of synthetic tailing regions include poly(A) sequences, poly(C) sequences, and polyA-G quartets. Non-limiting examples of chain terminating nucleosides include 2′-O methyl, F and locked nucleic acids (LNA). Additional suitable trailing sequences for inclusion in the mRNA are further described below.

The mRNA for administration may further be optimized with one or more chemical modifications to a naturally occurring ribonucleotide. For example, the chemical modification may be to an adenosine ribonucleoside, a cytidine ribonucleoside, a guanosine ribonucleoside, or a uridine ribonucleoside, or any combination thereof as described further herein. In some embodiments, the chemical modification comprises the partial or complete substitution of uridine ribonucleosides within the mRNA with a pseudouridine. In one embodiment, the pseudouridine is N1-methylpseudouridine. Other contemplated chemical modifications to the mRNA of the present invention are further described below.

The mRNA for administration can be administered alone or in a suitable pharmaceutical composition. For example, the mRNA can be formulated within a delivery vehicle for administration to a human to treat a Flavivirus infection. Any suitable delivery vehicle for human administration may be used. In non-limiting examples, the delivery vehicle may be a lipidoid formulation. In one embodiment, the delivery vehicle is a liposome, for example, a 3:1 mixture of 2,3-dioleyloxy-N-[2(sperminecarboxamido)ethyl]-N,N-dimethyl-1-propanaminium trifluoroacetate (DOSPA) and dioleylphosphatidylethanolamine (DOPE) or other suitable liposome. In another embodiment, the delivery vehicle can be a lipoplex. In another embodiment, the delivery vehicle is a lipid nanoparticle. In some embodiments, the delivery vehicle is a polymer. In some embodiments, the polymer is modified poly(ethyleneimine). In some embodiments, the delivery vehicle is a polymeric nanoparticle. Other suitable delivery vehicles for use are further described below.

The mRNA for administration may be administered to a human using any suitable administration route. In certain embodiments, the mRNA is administered by direct injection into the brain or testes. In some embodiments, the mRNA is administered by intrathecal injection. In some embodiments, the mRNA is administered via intravenous injection for delivery to infected monocytes or macrophages. In some embodiments, the mRNA is administered via intravenous injection in combination with targeted ultrasound therapy for delivery to the brain.

### BRIEF DESCRIPTION OF THE DRAWINGS

FIG. 1 is a schematic of an mRNA of the present invention.

FIG. 2 is a bar graph that shows the effect of either V5-Oas1b (V5-1b) mRNA or GFP mRNA on virus production by susceptible mouse C57BL/MEF cells, Huh7 human hepatocyte cells, and A549 human lung cells at 36 hours after infection. The cells were infected with West Nile virus, strain Eg101, at a MOI=1 and were transfected 6 hours later with either 1 μg of V5-1b mRNA or GFP mRNA. The y-axis is the virus titer measured in logarithmic plaque forming units (PFU) per millimeter. The x-axis is the cell type. Statistical analysis was performed using the student's t-test. *p<0.05. **p<0.01. ***p<0.0005.

FIG. 3 is a scatter diagram that shows the viral double stranded RNA levels at 36 hours after infection in susceptible mouse C57BL/6 MEF cells treated with GFP or V5-1b mRNA. C57BL/6 MEF cells were infected with West Nile virus, stain Eg101, at a MOI=1 and were transfected 6 hours later with either 1 μg of V5-1b mRNA or GFP mRNA. The y-axis is dsRNA levels measured as intensity per cell. The x-axis is the cell type. Statistical analysis was performed using the student's t-test. ****p<0.0005.

FIG. 4 is a scatter diagram that shows the viral double stranded RNA levels at 36 hours after infection in Huh7 human hepatocyte cells treated with GFP or V5-1b mRNA. Huh7 cells were infected with West Nile virus, strain Eg101, at a MOI=1 and were transfected 6 hours later with either 1 μg of V5-1b mRNA or GFP mRNA. The y-axis is dsRNA levels measured as intensity per cell. The x-axis is the cell type. Statistical analysis was performed using the student's t-test. ****p<0.0005.

FIG. 5 is a scatter diagram that shows the viral double stranded RNA levels at 36 hours after infection in A549 human lung cells treated with GFP or V5-1b mRNA. A549 cells were infected with West Nile virus, strain Eg101, at a MOI=1 and were transfected 6 hours later with either 1 μg of V5-1b mRNA or GFP mRNA. The y-axis is dsRNA levels measured as intensity per cell. The x-axis is the cell type. Statistical analysis was performed using the student's t-test. ****p<0.0005.

FIG. 6A is a cell panel of primary human astrocytes infected with West Nile virus, strain NY99, or Zika virus, strain PRVABC59, at a MOI of 2. At 6 hours after infection, the cells were transfected with either 1.5 ug V5-Oas1b mRNA or GFP mRNA. At 24 hours after infection, cells were processed for indirect immunofluorescence assay (IFA).

FIG. 6B is a graph showing virus titer in primary human astrocytes infected with West Nile virus, strain NY99, at a MOI of 2. At 6 hours after infection, the cells were transfected with either 1.5 ug V5-Oas1b mRNA or GFP mRNA. At 24 hours after infection, culture fluids were harvested and used to determine virus yield by plaque assay. Statistical analysis was performed using the student's t-test. ****p<0.001.

FIG. 6C is a cell panel of primary human monocytes infected with Dengue virus, strain 2 at a MOI of 2. At 6 hours after infection, the cells were transfected with either 1.5 ug V5-Oas1b mRNA or GFP mRNA. At 48 hours after infection, cells were processed for indirect immunofluorescence assay (IFA).

FIG. 6D is a graph showing virus titer in primary human monocytes infected with Dengue virus, strain 2, at a MOI of 2. At 6 hours after infection, the cells were transfected with either 1.5 ug V5-Oas1b mRNA or GFP mRNA. At 48 hours after infection, culture fluids were harvested and used to determine virus yield by plaque assay. Statistical analysis was performed using the student's t-test. ****p<0.001.

### DETAILED DESCRIPTION OF THE INVENTION

### Definitions

The compositions are described herein using standard nomenclature. Unless defined otherwise, all technical and scientific terms used herein have the same meaning as is commonly understood by one of skill in the art to which the invention belongs.

The terms “a” and “an” do not denote a limitation of quantity, but rather denote the presence of at least one of the referenced item. The term “or” means “and/or”. Recitation of ranges of values merely intend to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of example, or exemplary language (e.g. “such as”), is intended merely to better illustrate the invention and does not pose a limitation on the scope of the invention unless otherwise claimed.

The term “controlled release” as used herein refers to a pharmaceutical composition or compound release profile that conforms to a particular pattern of release to effect a therapeutic outcome.

An “effective amount” as used herein means an amount which provides a therapeutic or prophylactic benefit.

As used herein, “homology” refers to overall relatedness between polymeric molecules, e.g. between nucleic acid molecules (e.g. DNA molecules and/or RNA molecules) and/or polypeptide molecules. In some embodiments, polymeric molecules are “homologous” to one another if their sequences are at least about 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95, or even 99% identical or similar for at least about 20 amino acids. In accordance with the present invention, two protein sequences are considered to be homologous if the proteins are at least about 50%, 60%, 70%, 80%, 90%, 95, or even 99% identical or similar for at least about 20 amino acids.

The term “encapsulate” as used herein means to enclose, surround or encase. As it relates to the formulations of the mRNA of the present invention, encapsulation may be substantial, complete, or partial. The term “substantially encapsulated” means that at least greater than 50, 60, 70, 80, 85, 90, 95, 96, 97, 98, 99, 99.9, 99.99, or greater than 99.999% of the pharmaceutical composition or compound of the invention may be enclosed, surrounded, or encased within the delivery vehicle. “Partially encapsulated” means that less than 10, 20, 30, 40, 50, or less of the pharmaceutical composition or compound of the invention may be enclosed, surrounded, or encased within the delivery vehicle. Advantageously, encapsulation may be determined by measuring the escape or the activity of the pharmaceutical composition or compound of the present invention by using fluorescence and/or electron micrography. For example, at least 1, 5, 10, 20, 30, 40, 50, 60, 70, 80, 90, 95, 96, 97, 98, 99, 99.9, 99.99, or greater than 99.999% of the pharmaceutical composition or compound of the invention are encapsulated in the delivery vehicle.

The term “messenger RNA” (mRNA) as used herein refers to any polynucleotide which encodes the rOas1b polypeptide of interest, or variants thereof as described herein, and which is capable of being translated to produce the encoded polypeptide in vitro, in vivo, in situ or ex vivo.

A “microRNA (miRNA) binding site” represents a nucleotide location or region of a nucleic acid transcript to which at least the seed region of a miRNA binds. It should be understood that “binding” may follow traditional Watson-Crick hybridization rules or may reflect any stable association of the microRNA with the target sequence at or adjacent to the microRNA site.

As used herein “modified” refers to a changed state or structure of a molecule of the invention. Molecules may be modified in many ways including chemically, structurally, and functionally. In one embodiment, the polynucleotides of the present invention are “chemically modified” by the introduction of non-natural nucleosides and/or nucleotides, e.g., as it relates to the natural ribonucleotides A, U, G, and C. Modifications of the nucleosides and/or nucleotides as used in the present invention may be naturally occurring (i.e. comprise a nucleotide and/or nucleoside other than the natural ribonucleotides A, U, G, and C) or may be artificial. Non-canonical nucleotides such as the cap structures are not considered “modified” although they differ from the chemical structure of A, G, C, and U ribonucleotides. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. When the polynucleotides of the present invention are chemically and/or structurally modified, the polynucleotides may be referred to as “modified nucleotides”.

As used herein, “operably linked,” when referring to a first nucleic acid sequence that is operably linked with a second nucleic acid sequence, means a situation when the first nucleic acid sequence is placed in a functional relationship with the second nucleic acid sequence.

As used herein, “pseudouridine” refers to the C-glycoside isomer of the nucleoside uridine. A “pseudouridine analog” is any modification, variant, isoform or derivative of uridine. For example, pseudouridine analogs include, but are not limited to, 1-carboxymethyl-pseudouridine, 1-propynyl-pseudouridine, 1-taurinomethyl-pseudouridine, 1-taurinomethyl-4-thio-pseudouridine, 1-methylpseudouridine, 1-methyl-4-thio-pseudouridine, 3-methylpseudouridine, 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 2-methoxyuridine, 2-methoxy-4-thio-uridine, 4-methoxy-pseudouridine, 4-methoxy-2-thio-pseudouridine, N1-methyl-pseudouridine, 1-methyl-3-(3-amino-3-carboxypropyl)pseudouridine, and 2′-O-methyl-pseudouridine.

The term “sustained release” as used herein refers to a pharmaceutical composition or compound release profile that conforms to a release rate over a specific period of time.

The term “synthetic” means produced, prepared, and/or manufactured by the hand of man. Synthesis of polynucleotides or other molecules of the present invention may be chemical or enzymatic.

As used herein, the term “transfection” refers to methods to introduce exogenous nucleic acids into a cell. Methods of transfection include, but are not limited to, chemical methods, physical treatments, cationic lipids or mixtures, or the like.

As used herein, “translation” is the process by which mRNA is processed by a ribosome or ribosomal-like machinery, e.g., cellular or artificial, to produce a peptide or polypeptide.

As used herein, “unmodified” refers to any substance, compound, or molecule prior to being changed in any way. Unmodified may, but does not always, refer to the wild type or native form of a biomolecule. Molecules may undergo a series of modifications whereby each modified molecule may serve as the “unmodified” starting molecule for a subsequence modification.

As used herein, a “unit dose” refers to a discrete amount of the pharmaceutical composition comprising a predetermined amount of the mRNA described herein.

To “treat” a disease as the term is used herein means to reduce the frequency or severity of at least one sign or symptom of a disease of disorder experienced by a human “i.e. a palliative treatment” or to decrease a cause or effect of the disease or disorder (i.e. disease-modifying treatment).

As used herein, “pharmaceutical compositions” are compositions comprising the mRNA described herein and at least one other substance, such as a carrier or delivery vehicle.

The term “carrier” applied to pharmaceutical combinations of the invention refers to a diluent, excipient, or vehicle with which an active compound is provided.

A “pharmaceutically acceptable excipient” means an excipient that is useful in preparing a pharmaceutical composition that is generally safe, non-toxic and neither biologically nor otherwise inappropriate for administration to a human.

Messenger RNA (mRNA) Architecture

According to the present invention, the mRNA provided herein encode resistant 5′2′-5′ oligoadenylate synthetase 1b (rOas1b) from Mus musculus (UniProt KB—Q60856) and natural or artificial variants thereof. They may have any of the features described herein.

Traditionally, the basic components of an mRNA molecule include at least a coding region, a 5′ untranslated region (5′ UTR), a 3′ untranslated region (3′ UTR), a 5′ terminal cap and a polyadenylate tail. The mRNA of the present invention may include one or more modifications from the naturally occurring rOAS1b mRNA transcript. The modified mRNAs of the present invention are distinguished from wild-type mRNA in their functional and/or structural design features, which may serve to overcome existing problems of effective polypeptide production using nucleic-acid based therapeutics, while still maintaining anti-Flavivirus activity. It is to be understood that the murine rOas1b peptide, and natural or artificial variants thereof, may be expressed in a human cell using the modified mRNA as described herein.

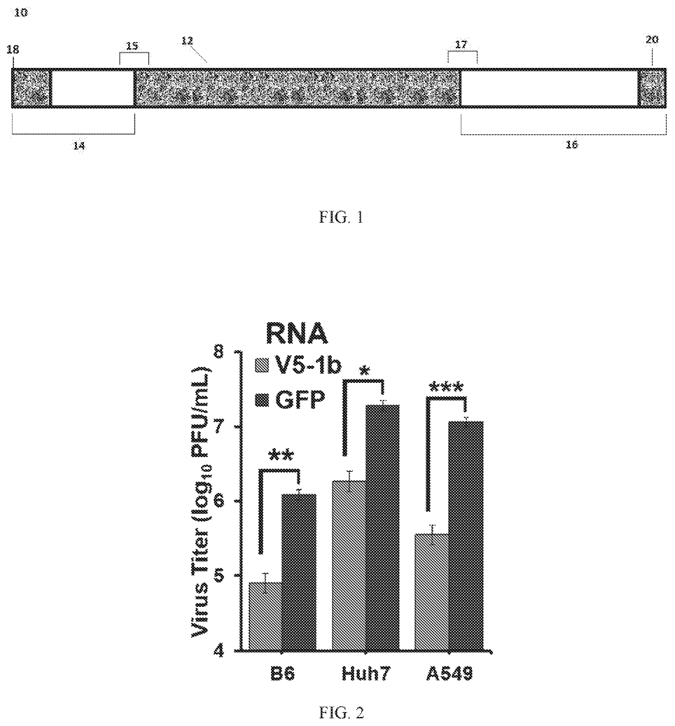

As shown in FIG. 1, the mRNA 10 described herein contains a first region of linked nucleotides 12 that is operably linked to a first flanking region 14 on the 5′ end and a second flanking region 16 at the 3′ end. In typical embodiments, the region 12 comprises the rOAS1b encoding sequence. The first flanking region 14 may comprise a region of linked nucleotides comprising one or more 5′ UTR sequences. The first flanking region may include at least one nucleic acid sequence including for example, translation control sequences, for example a Kozak consensus sequence. The first flanking region may also include a 5′ terminal cap at the terminal 5′ base location 18. The 5′ terminal capping region 18 may include a naturally occurring cap, a synthetic cap, or an optimized cap. In certain embodiments, the 5′ cap is enzymatically added. Non-limiting examples of optimized caps include the caps taught by Rhoads in U.S. Pat. No. 7,074,596 and International Patent Publication No. WO2008157668, WO2009149253 and WO2013103659, the contents of each of which are herein incorporated by reference in their entirety. The second flanking region 16 may comprise a region of linked nucleotides comprising one or more 3′ UTR sequences. The second flanking region 16 may include at least one nucleic acid sequence including, but not limited to, translation control sequences. The second flanking region 16 may also comprise a 3′ tailing sequence 20. The 3′ tailing sequence 20 may contain a polyadenylation motif or short poly A tail, for example less than about 100 nucleotides. Bridging the 5′ terminus of the first region and the first flanking region is a first operational region 15. Traditionally this operational region comprises a Start codon. The operational region may alternatively comprise any translation control sequence, for example a Kozak consensus sequence, or signal including a Start codon.

Bridging the 3′ terminus of the first region and the second flanking region is a second operational region 17. Traditionally this operational region comprises a Stop codon. The operational region may alternatively comprise any translation control sequence or signal including a Stop codon. Multiple serial stop codons may also be used in the polynucleotide. In one embodiment, the operational region of the present invention may comprise two stop codons. The first stop codon may be “UGA” and the second stop codon may be selected from the group consisting of “UAA,” “UGA” or “UAG.”

rOAS1b Coding Region

The present invention provides an mRNA comprising a coding region encoding murine resistant 2′-5′ oligoadenylate synthetase 1b (rOas1b) or a variant thereof that is 80%, 85%, 90%, 95%, 96%, 97%, 98%, 99% or more homologous.

In one embodiment, the coding region comprises a polynucleotide sequence encoding the polypeptide of SEQ. ID. NO.: 1 (UniProt KB—Q60856), or a variant thereof that is 80%, 85%, 90%, 95%, 96%, 97%, 98%, 99% or more homologous:

(SEQ. ID. NO.: 1)
MEQDLRSIPASKLDKFIENHLPDTSFCADLREVIDALCALLKDRSFRGPVR
RMRASKGVKGKGTTLKGRSDADLVVFLNNLTSFEDQLNQQGVLIKEIKKQL
CEVQHERRCGVKFEVHSLRSPNSRALSFKLSAPDLLKEVKFDVLPAYDLLD
HLNILKKPNQQFYANLISGRTPPGKEGKLSICFMGLRKYFLNCRPTKLKRL
IRLVTHWYQLCKEKLGDPLPPQYALELLTVYAWEYGSRVTKFNTAQGFRTV
LELVTKYKQLQIYWTVYYDFRHQEVSEYLHQQLKKDRPVILDPADPTRNIA
GLNPKDWRRLAGEAAAWLQYPCFKYRDGSSVCSWEVPTEVGVPMKYLLCRI
FWLLFWSLFHFIFGKTSSG.

In another embodiment, the coding region comprises a polynucleotide sequence encoding a polypeptide of SEQ. ID. NO.: 1 with an A36S substitution, S45F substitution, R47Q substitution, V50G substitution, G63C substitution, T65A substitution, S83Y substitution, Q90R substitution, C103Y substitution, V105I substitution, C111F substitution, H118Q substitution, L151V substitution, P176L substitution, K181E substitution, S183L substitution, I184T substitution, R190Q substitution, R206H substitution, Q266R substitution, H277L substitution, Q278P substitution, D291V substitution, A299V substitution, I305V substitution, A322T substitution, S336P substitution, G347A substitution, M350T substitution, L354F substitution, F368L substitution, or combinations thereof. In one embodiment, the coding region encodes a polypeptide of SEQ. ID. NO.: 1 with an A36S substitution. In one embodiment, the coding region encodes a polypeptide of SEQ. ID. NO.: 1 with a S45F substitution. In one embodiment, the coding region encodes a polypeptide of SEQ ID. NO.: with a R47Q substitution. In one embodiment, the coding region encodes a polypeptide of SEQ. ID. NO.: 1 with a V50G substitution. In one embodiment, the coding region encodes a polypeptide of SEQ. ID. NO.: 1 with a G63C substitution. In one embodiment, the coding region encodes a polypeptide of SEQ. ID. NO.: 1 with a T65A substitution. In one embodiment, the coding region encodes a polypeptide of SEQ. ID. NO.: 1 with a S83Y substitution. In one embodiment, the coding region encodes a polypeptide of SEQ. ID. NO.: 1 with a Q90R substitution. In one embodiment, the coding region encodes a polypeptide of SEQ. ID. NO.: 1 with a C103Y substitution. In one embodiment, the coding region encodes a polypeptide of SEQ. ID. NO.: 1 with a V105I substitution. In one embodiment, the coding region encodes a polypeptide of SEQ. ID. NO.: 1 with a C111F substitution. In one embodiment, the coding region encodes a polypeptide of SEQ. ID. NO.: 1 with a Hi 18Q substitution. In one embodiment, the coding region encodes a polypeptide of SEQ. ID. NO.: 1 with a L151V substitution. In one embodiment, the coding region encodes a polypeptide of SEQ. ID. NO.: 1 with a P176L substitution. In one embodiment, the coding region encodes a polypeptide of SEQ. ID. NO.: 1 with a K181E substitution. In one embodiment, the coding region encodes a polypeptide of SEQ. ID. NO.: 1 with a S183L substitution. In one embodiment, the coding region encodes a polypeptide of SEQ. ID. NO.: 1 with an I184T substitution. In one embodiment, the coding region encodes a polypeptide of SEQ. ID. NO.: 1 with a R190Q substitution. In one embodiment, the coding region encodes a polypeptide of SEQ. ID. NO.: 1 with a R206H substitution. In one embodiment, the coding region encodes a polypeptide of SEQ. ID. NO.: 1 with a Q266R substitution. In one embodiment, the coding region encodes a polypeptide of SEQ. ID. NO.: 1 with a H277L substitution. In one embodiment, the coding region encodes a polypeptide of SEQ. ID. NO.: 1 with a Q278P substitution. In one embodiment, the coding region encodes a polypeptide of SEQ. ID. NO.: 1 with a D291V substitution. In one embodiment, the coding region encodes a polypeptide of SEQ. ID. NO.: 1 with an A299V substitution. In one embodiment, the coding region encodes a polypeptide of SEQ. ID. NO.: 1 with an I305V substitution. In one embodiment, the coding region encodes a polypeptide of SEQ. ID. NO.: 1 with an A322T substitution. In one embodiment, the coding region encodes a polypeptide of SEQ. ID. NO.: 1 with a S336P substitution. In one embodiment, the coding region encodes a polypeptide of SEQ. ID. NO.: 1 with a G347A substitution. In one embodiment, the coding region encodes a polypeptide of SEQ. ID. NO.: 1 with a M350T substitution. In one embodiment, the coding region encodes a polypeptide of SEQ. ID. NO.: 1 with a L354F substitution. In one embodiment, the coding region encodes a polypeptide of SEQ. ID. NO.: 1 with a F368L substitution.

In one embodiment, the coding region encodes a polypeptide of SEQ. ID. NO.: 1 with an A36S substitution, a G63C substitution, a T65A substitution, a S83Y substitution, a L151V substitution, a S183L substitution, an I184T substitution, a Q266R substitution, a H277L substitution, an M350T substitution, and an L354F substitution.

In one embodiment, the coding region encodes a polypeptide of SEQ. ID. No.: 1 with an A36S substitution, an R47Q substation, a V50G substitution, a G63C substitution, an S83Y substitution, a Q90R substitution, a V105I substitution, a C111F substitution, a K181E substitution, an I184T substitution, Q266R substitution, a Q278P substitution, a D291V substitution, an S336P substitution, a G347A substitution, and an L354F substitution.

In one embodiment, the coding region encodes a polypeptide of SEQ. ID. No.: 1 with an A36S substitution, a G63C substitution, a S83Y substitution, a C111F substitution, an H118Q substitution, an L151V substitution, a Q266R substitution, an A299V substitution, an I305V substitution, an S336P substitution, an L354F substitution, and an F368L substitution.

In one embodiment, the coding region encodes a polypeptide of SEQ. ID. No.: 1 with an S45F substitution, a G63C substitution, a T65A substitution, S83Y substitution, a C103Y substitution, a C111F substitution, an H118Q substitution, a P176L substitution, an S183L substitution, an I184T substitution, an R206H substitution, a Q266R substitution, an S336P substitution, a G347A substitution, and an L354F substitution.

In one embodiment, the coding region encodes a polypeptide of SEQ. ID. NO.: 1 with a T65A substitution and an R190Q substitution.

In one embodiment, the coding region encodes a polypeptide of SEQ. ID. NO.: 1 with a Q266R substitution, an A322T substitution, an S336P substitution, and an L354F substitution.

In one embodiment, the coding region comprises a polynucleotide sequence encoding a polypeptide of SEQ. ID. NO.: 2, or a variant thereof that is at least about 80%, 85%, 90%, 95%, 96%, 97%, 98%, 99% or more homologous: (SEQ. ID. NO.: 2)

(SEQ. ID. NO.: 2)
MEQDLRSIPASKLDKFIENHLPDTSFCADLREVIDALCALLKDRSFRGPVR
RMRASKGVKGKGTTLKGRSDADLVVFLNNLTSFEDQLNQQGVLIKEIKKQL
CEVQHERRCGVKFEVHSLRSPNSRALSFKLSAPDLLKEVKFDVLPAYDLLD
HLNILKKPNQQFYANLISGRTPPGKEGKLSICFMGLRKYFLNCRPTKLKRL
IRLVTHWYQLCKEKLGDPLPPQYALELLTVYAWEYGSRVTKFNTAQGFRTV
LELVTKYKQLRIYWTVYYDFRHQEVSEYLHQQLKKDRPVILDPADPTRNIA
GLNPKDWRRLAGEAATWLQYPCFKYRDGSPVCSWEVPTEVGVPMKYLFCRI
FWLLFWSLFHFIFGKTSSG.

In some embodiments, the coding region comprises a polynucleotide sequence encoding a polypeptide of SEQ. ID No.: 3, or a variant thereof that is at least about 80%, 85%, 90%, 95%, 96%, 97%, 98%, 99% or more homologous:

(SEQ. ID. NO.: 3)
MEQDLRSIPASKLDKFIENHLPDTSFCADLREVIDX¹LCALLKDRX²FX³G
PX⁴RRMRASKGVKGKX⁵TX⁶LKGRSDADLVVFLNNLTX⁷FEDQLNX⁸QGVL
IKEIKKQLX⁹EX¹⁰QHERRX¹¹GVKFEVX¹²SLRSPNSRALSFKLSAPDLL
KEVKFDVLPAYDX¹³LDHLNILKKPNQQFYANLISGRTPX¹⁴GKEGX¹⁵L
X¹⁶X¹⁷CFMGLX¹⁸KYFLNCRPTKLKRLIX¹⁹LVTHWYQLCKEKLGDPLPP
QYALELLTVYAWEYGSRVTKFNTAQGFRTVLELVTKYKQLX²⁰IYWTVYYD
FRX²¹X²²EVSEYLHQQLKKX²³RPVILDPX²⁴DPTRNX²⁵AGLNPKDWRR
LAGEAAX²⁶WLQYPCFKYRDGSX²⁷VCSWEVPTEVX²⁸VPX²⁹KYLX³⁰CR
IFWLLFWSLX³¹HFIFGKTSSG,

wherein X¹ to X³¹ are as defined in Table 1 below:

TABLE 1

| Variable | Amino Acid |
|---|---|
| X¹ | A or S |
| X² | F or S |
| X³ | Q or F |
| X⁴ | G or V |
| X⁵ | C or G |
| X⁶ | A or T |
| X⁷ | S or Y |
| X⁸ | Q or R |
| X⁹ | Y or C |
| X¹⁰ | I or V |
| X¹¹ | C or F |
| X¹² | H or Q |
| X¹³ | L or V |
| X¹⁴ | L or P |
| X¹⁵ | E or K |
| X¹⁶ | L or S |
| X¹⁷ | I or T |
| X¹⁸ | R or Q |
| X¹⁹ | R or H |
| X²⁰ | Q or R |
| X²¹ | H or L |
| X²² | Q or P |
| X²³ | D or V |
| X²⁴ | A or V |
| X²⁵ | I or V |
| X²⁶ | A or T |
| X²⁷ | S or P |
| X²⁸ | G or A |
| X²⁹ | M or T |
| X³⁰ | L or F |
| X³¹ | F or L |

In one embodiment, X¹ is A. In one embodiment, X¹ is S. In one embodiment, X² is F. In one embodiment, X² is S. In one embodiment, X³ is Q. In one embodiment, X³ is F. In one embodiment, X⁴ is G. In one embodiment, X⁴ is V. In one embodiment, X⁵ is C. In one embodiment, X⁵ is G. In one embodiment, X⁶ is A. In one embodiment, X⁶ is T. In one embodiment, X⁷ is S. In one embodiment, X⁷ is Y. In one embodiment, X⁸ is Q. In one embodiment, X⁸ is R. In one embodiment, X⁹ is Y. In one embodiment, X⁹ is C. In one embodiment, X¹⁰ is I. In one embodiment, X¹⁰ is V. In one embodiment, X¹¹ is C. In one embodiment, X¹¹ is F. In one embodiment, X¹² is H. In one embodiment, X¹² is Q. In one embodiment, X¹³ is L. In one embodiment, X¹³ is V. In one embodiment, X¹⁴ is L. In one embodiment, X¹⁴ is P. In one embodiment, X¹⁵ is E. In one embodiment, X¹⁵ is K. In one embodiment, X¹⁶ is L. In one embodiment, X¹⁶ is S. In one embodiment, X¹⁷ is I. In one embodiment, X¹⁷ is T. In one embodiment, X¹⁸ is R. In one embodiment, X¹⁸ is Q. In one embodiment, X¹⁹ is R. In one embodiment, X¹⁹ is H. In one embodiment, X²⁰ is Q. In one embodiment, X²⁰ is R. In one embodiment, X²¹ is H. In one embodiment, X²¹ is L. In one embodiment, X²² is Q. In one embodiment, X²² is P. In one embodiment, X²³ is D. In one embodiment, X²³ is V. In one embodiment, X²⁴ is A. In one embodiment, X²⁴ is V. In one embodiment, X²⁵ is I. In one embodiment, X²⁵ is V. In one embodiment, X²⁶ is A. In one embodiment, X²⁶ is T. In one embodiment, X²⁷ is S. In one embodiment, X²⁷ is P. In one embodiment, X²⁸ is G. In one embodiment, X²⁸ is A. In one embodiment, X²⁹ is M. In one embodiment, X²⁹ is T. In one embodiment, X³⁰ is L. In one embodiment, X³⁰ is G. In one embodiment, X³¹ is F. In one embodiment, X³¹ is L.

In one embodiment, the coding region encodes a polypeptide of SEQ. ID. NO.: 3 wherein X¹ is S, X⁵ is C, X⁶ is A, X⁷ is Y, X¹³ is V, X¹⁶ is L, X¹⁷ is T, X²⁰ is R, X²¹ is L, X²⁹ is T, and X³⁰ is F.

In one embodiment, the coding region encodes a polypeptide of SEQ. ID. No.: 3 wherein X¹ is S, X³ is Q, X⁴ is G, X⁵ is C, X⁷ is Y, X⁸ is R, X¹⁰ is I, X¹¹ is F, X¹⁵ is E, X¹⁷ is T, X²⁰ is R, X²² is P, X²³ is V, X²⁷ is P, X²⁸ is A, and X³⁰ is F.

In one embodiment, the coding region encodes a polypeptide of SEQ. ID. No.: 3 wherein X¹ is S, X⁵ is C, X⁷ is Y, X¹¹ is F, X¹² is Q, X¹³ is V, X²⁰ is R, X²⁴ is V, X²⁵ is V, X²⁷ is P, X³⁰ is F, and X³¹ is L.

In one embodiment, the coding region encodes a polypeptide of SEQ. ID. No.: 3 wherein X² is F, X⁵ is C, X⁶ is A, X⁷ is Y, X⁹ is Y, X¹¹ is F, X¹² is Q, X¹⁴ is L, X¹⁶ is L, X¹⁷ is T, X¹⁹ is H, X²⁰ is R, X²⁷ is P, X²⁸ is A, and X³⁰ is F.

In one embodiment, the coding region encodes a polypeptide of SEQ. ID. NO.: 3 wherein X⁶ is A and X¹⁸ is Q.

In one embodiment, the coding region encodes a polypeptide of SEQ. ID. NO.: 3 wherein X²⁰ is R, X²⁶ is T, X²⁷ is P, and X³⁰ is F.

In one embodiment, the mRNA comprises a coding sequence of SEQ. ID. NO.: 4, or a variant thereof that is 80%, 85%, 90%, 95%, 96%, 97%, 98%, 99% or more homologous:

(SEQ. ID. NO.: 4)
AUGGAGCAAGAUCUUAGAUCCAUACCAGCUUCCAAGCUCGAUAAGUUCAUU
GAGAAUCAUCUGCCCGACACCUCCUUUUGCGCUGACCUUAGAGAGGUCAUA
GAUGCCUUGUGCGCCCUUCUCAAAGACCGAUCAUUUAGAGGUCCUGUCCGG
CGCAUGAGAGCAUCAAAGGGGGUCAAGGGUAAAGGGACCACACUUAAGGGG
AGGUCUGACGCCGACCUGGUCGUCUUCCUCAACAACUUGACCAGCUUUGAG
GAUCAGCUCAAUCAACAAGGUGUACUUAUAAAAGAGAUCAAAAAACAACUC
UGUGAAGUGCAGCACGAACGCAGGUGUGGUGUUAAGUUUGAAGUUCAUUCA
CUUCGCAGUCCCAAUUCUCGGGCUCUCAGCUUUAAGCUGUCAGCCCCCGAC
UUGCUCAAAGAAGUGAAGUUUGAUGUGCUGCCCGCAUACGACCUUCUCGAC
CAUCUCAACAUACUGAAGAAACCAAACCAACAGUUUUACGCAAACCUCAUU
AGCGGACGAACUCCACCUGGCAAAGAGGGGAAAUUGAGCAUAUGUUUCAUG
GGACUUCGGAAGUACUUCCUGAACUGCCGCCCCACUAAACUGAAGAGGCUU
AUUAGGCUCGUCACUCACUGGUAUCAAUUGUGUAAGGAGAAGCUGGGGGAU
CCCCUCCCACCUCAAUACGCACUUGAACUCUUGACCGUUUACGCAUGGGAG
UACGGAAGUCGGGUCACUAAAUUCAAUACAGCUCAGGGCUUUCGGACCGUG
UUGGAACUCGUAACUAAGUAUAAGCAGUUGAGGAUAUACUGGACUGUAUAU
UAUGAUUUUAGACACCAAGAGGUUUCAGAGUACCUUCAUCAACAGCUUAAA
AAAGAUCGACCCGUUAUUCUGGACCCUGCCGACCCCACACGAAAUAUCGCC
GGCCUUAACCCUAAGGACUGGAGACGACUUGCUGGGGAAGCAGCCACUUGG
CUUCAGUAUCCCUGCUUCAAAUAUAGAGACGGCAGCCCUGUGUGUUCUUGG
GAAGUCCCCACAGAGGUCGGGGUCCCCAUGAAAUAUCUCUUCUGUCGCAUC
UUCUGGCUCUUGUUUUGGAGUCUGUUCCACUUCAUCUUUGGUAAAACCUCC
UCAGGUUAGUGAUAA.

### Chemical Modification of Nucleotides

The mRNA of the present invention includes at least one chemical modification. As used herein, the terms “chemical modification” or “chemically modified” refer to modification with respect to adenosine (A), guanosine (G), uridine (U), or cytidine (C) ribonucleosides in one or more of their position, pattern, percent, or population. Generally, these terms are not intended to refer to modifications in naturally occurring 5′-terminal mRNA cap moieties.

The chemical modifications may be various distinct modifications. In some embodiments, the mRNA may contain one, two, or more of the same or different nucleoside or nucleotide chemical modifications. In some embodiments, a modified mRNA may exhibit reduced degradation in the cell, as compared to an unmodified polynucleotide. Chemical modifications to the nucleosides as used in the present invention may be naturally occurring or may be artificial, i.e. not found in nature and synthesized by man.

In some embodiments, the one or more chemical modifications include modifications to an adenosine ribonucleoside within the mRNA. Representative examples of adenosine ribonucleoside modifications include, but are not limited to 2-methylthio-N6-(cishydroxyisopentenyl)adenosine (ms2i6A), 2-methylthio-N6-methyladenosine (ms2m6A), 2-methylthio-N6-threonylcarbamoyladenosine (ms2t6A), N6-glycinylcarbamoyladenosine (g6A), N6-isopentenyladenosine (i6A), N6-methyladenosine (m6A), N6-threonylcarbamoyladenosine (t6A), 1,2′-O-dimethyladenosine (m1Am), 1-methyladenosine (m1A), 2′-O-methyladenosine (Am), 2′-O-ribosyladenosine (phosphate) (Ar(p)), 2-methyladenosine (m2A), 2-methylthio-N6 isopentenyladenosine (ms2i6A), 2-methylthio-N6-hydroxynorvalylcarbamoyladenosine (ms2hn6A), 2′-O-methyladenosine (m6A), isopentenyladenosine (Iga), N6-(cis-hydroxyisopentenyl)adenosine (io6A), N6,2′-O-dimethyladenosine (m6Am), N6,N6,2′-O-trimethyladenosine (m62Am), N6,N6-dimethyladenosine (m62A), N6-acetyladenosine (ac6A), N6-hydroxynorvalylcarbamoyladenosine (hn6A), N6-methyl-N6-threonylcarbamoyladenosine (m6t6A), 2-methyladenosine (m2A), 2-methylthio-N-isopentenyladenosine (ms2i6A), 7-deaza-adenosine, N1-methyl-adenosine, N6,N6(dimethyl)adenine, N6-cis-hydroxy-isopentenyl-adenosine, α-thio-adenosine, 2(amino)adenine, 2(aminopropyl)adenine, 2(methylthio)N6(isopentenyl)adenine, 2-(alkyl)adenine, 2-(aminoalkyl)adenine, 2-(aminopropyl)adenine, 2-(halo)adenine, 2-(propyl)adenine, 2′-amino-2′-deoxy-ATP, 2′-azido-2′-deoxy-ATP, 2′-deoxy-2′-a-aminoadenosine TP, 2′-deoxy-2′-a-azidoadenosine TP, 6(alkyl)adenine, 6(methyl)adenine, 6-(alkyl)adenine, 6-(methyl)adenine, 7(deaza)adenine, 8(alkenyl)adenine, 8(alkynyl)adenine, 8(amino)adenine, 8(thioalkyl)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, 8-azido-adenosine, azaadenine, deazaadenine, N6(methyl)adenine, N6-(isopentyl)adenine, 7-deaza-8-aza-adenosine, 7-methyladenine, 1-deazaadenosine TP, 2′-fluoro-N6-Bz-deoxyadenosine TP, 2′-OMe-2-Amino-ATP, 2′-O-methyl-N6-Bz-deoxyadenosine TP, 2′-a-ethynyladenosine TP, 2-aminoadenine, 2-aminoadenosine TP, 2-amino-ATP, 2′-a-trifluoromethyladenosine TP, 2-azidoadenosine TP, 2′-b-ethynyladenosine TP, 2-bromoadenosine TP, 2′-b-trifluoromethyladenosine TP, 2-chloroadenosine TP, 2′-deoxy-2′,2′-difluoroadenosine TP, 2′-deoxy-2′-a-mercaptoadenosine TP, 2′-deoxy-2′-a-thiomethoxyadenosine TP, 2′-deoxy-2′-b-aminoadenosine TP, 2′-deoxy-2′-b-azidoadenosine TP, 2′-deoxy-2′-b-bromoadenosine TP, 2′-deoxy-2′-b-chloroadenosine TP, 2′-deoxy-2′-b-fluoroadenosine TP, 2′-deoxy-2′-b-iodoadenosine TP, 2′-deoxy-2′-b-mercaptoadenosine TP, 2′-deoxy-2′-b-thiomethoxyadenosine TP, 2-fluoroadenosine TP, 2-iodoadenosine TP, 2-mercaptoadenosine TP, 2-methoxy-adenine, 2-methylthio-adenine, 2-trifluoromethyladenosine TP, 3-deaza-3-bromoadenosine TP, 3-Deaza-3-chloroadenosine TP, 3-Deaza-3-fluoroadenosine TP, 3-Deaza-3-iodoadenosine TP, 3-deazaadenosine TP, 4′-azidoadenosine TP, 4′-carbocyclic adenosine TP, 4′-ethynyladenosine TP, 5′-homo-adenosine TP, 8-aza-ATP, 8-bromo-adenosine TP, 8-trifluoromethyladenosine TP, 9-deazaadenosine TP, 2-aminopurine, 7-deaza-2,6-diaminopurine, 7-deaza-8-aza-2,6-diaminopurine, 7-deaza-8-aza-2-aminopurine, 2,6-diaminopurine, 7-deaza-8-aza-adenine, and 7-deaza-2-aminopurine.

In one embodiment, from about 5% to 100% of the adenosine ribonucleosides within the mRNA are modified. In one embodiment, from about 5%, about 10%, about 15%, about 20%, about 30%, about 40%, about 50%, about 60%, about 70%, about 80%, about 90%, or about 100% of the adenosine ribonucleosides within the mRNA are modified.

In some embodiments, the one or more chemical modifications include modifications to a cytidine ribonucleoside within the mRNA. Representative examples of cytidine ribonucleoside modifications include, but are not limited to, 2-thiocytidine (s2C), 3-methylcytidine (m3C), 5-formylcytidine (f5C), 5-hydroxymethylcytidine (hm5C), 5-methylcytidine (m5C), N4-acetylcytidine (ac4C), 2′-O-methylcytidine (Cm), 5,2′-O-dimethylcytidine (m5 Cm), 5-formyl-2′-O-methylcytidine (f5Cm), lysidine (k2C), N4,2′-O-dimethylcytidine (m4Cm), N4-acetyl-2′-O-methylcytidine (ac4Cm), N4-methylcytidine (m4C), N4,N4-dimethyl-2′-OMe-Cytidine TP, 4-methylcytidine, 5-aza-cytidine, pseudo-iso-cytidine, pyrrolo-cytidine, α-thio-cytidine, 2-(thio)cytosine, 2′-amino-2′-deoxy-CTP, 2′-azido-2′-deoxy-CTP, 2′-deoxy-2′-a-aminocytidine TP, 2′-deoxy-2′-a-azidocytidine TP, 3(deaza)5(aza)cytosine, 3(methyl)cytosine, 3-(alkyl)cytosine, 3-(deaza)5(aza)cytosine, 3-(methyl)cytidine, 4,2′-O-dimethylcytidine, 5(halo)cytosine, 5(methyl)cytosine, 5(propynyl)cytosine, 5(trifluoromethyl)cytosine, 5-(alkyl)cytosine, 5-(alkynyl)cytosine, 5-(halo)cytosine, 5-(propynyl)cytosine, 5-(trifluoromethyl)cytosine, 5-bromo-cytidine, 5-iodo-cytidine, 5-propynyl cytosine, 6-(azo)cytosine, 6-aza-cytidine, azacytosine, deazacytosine, N4(acetyl)cytosine, 1-methyl-1-deaza-pseudoisocytidine, 1-methyl-pseudoisocytidine, 2-methoxy-5-methyl-cytidine, 2-methoxy-cytidine, 2-thio-5-methyl-cytidine, 4-methoxy-1-methyl-pseudoisocytidine, 4-methoxy-pseudoisocytidine, 4-thio-1-methyl-1-deaza-pseudoisocytidine, 4-thio-1-methyl-pseudoisocytidine, 4-thio-pseudoisocytidine, 5-aza-zebularine, 5-methyl-zebularine, pyrrolo-pseudoisocytidine, zebularine, (E)-5-(2-bromo-vinyl)cytidine TP, 2,2′-anhydro-cytidine TP hydrochloride, 2′-fluor-N4-Bz-cytidine TP, 2′-fluoro-N4-Acetyl-cytidine TP, 2′-O-methyl-N4-acetyl-cytidine TP, 2′-O-methyl-N4-Bz-cytidine TP, 2′-a-ethynylcytidine TP, 2′-a-trifluoromethylcytidine TP, 2′-b-ethynylcytidine TP, 2′-b-trifluoromethylcytidine TP, 2′-deoxy-2′,2′-difluorocytidine TP, 2′-deoxy-2′-a-mercaptocytidine TP, 2′-deoxy-2′-a-thiomethoxycytidine TP, 2′-deoxy-2′-b-aminocytidine TP, 2′-deoxy-2′-b-azidocytidine TP, 2′-deoxy-2′-b-bromocytidine TP, 2′-deoxy-2′-b-chlorocytidine TP, 2′-deoxy-2′-b-fluorocytidine TP, 2′-deoxy-2′-b-iodocytidine TP, 2′-deoxy-2′-b-mercaptocytidine TP, 2′-deoxy-2′-b-thiomethoxycytidine TP, 2′-O-methyl-5-(1-propynyl)cytidine TP, 3′-ethynylcytidine TP, 4′-szidocytidine TP, 4′-carbocyclic cytidine TP, 4′-ethynylcytidine TP, 5-(1-propynyl)ara-cytidine TP, 5-(2-chloro-phenyl)-2-thiocytidine TP, 5-(4-amino-phenyl)-2-thiocytidine TP, 5-aminoallyl-CTP, 5-cyanocytidine TP, 5-ethynylara-cytidine TP, 5-ethynylcytidine TP, 5′-homo-cytidine TP, 5-methoxycytidine TP, 5-trifluoromethyl-cytidine TP, N4-amino-cytidine TP, N4-benzoyl-cytidine TP, and pseudoisocytidine.

In one embodiment, from about 5% to 100% of the cytidine ribonucleosides within the mRNA are modified. In one embodiment, from about 5%, about 10%, about 15%, about 20%, about 30%, about 40%, about 50%, about 60%, about 70%, about 80%, about 90%, or about 100% of the cytidine ribonucleosides within the mRNA are modified.

In some embodiments, the one or more chemical modifications include modifications to a guanosine ribonucleoside within the mRNA. Representative examples of guanosine ribonucleoside modifications include, but are not limited to, 7-methylguanosine (m7G), N2,2′-O-dimethylguanosine (m2Gm), N2-methylguanosine (m2G), wyosine (imG), 1,2′-O-dimethylguanosine (m1Gm), 1-methylguanosine (m1G), 2′-O-methylguanosine (Gm), 2′-O-ribosylguanosine (phosphate) (Gr(p)), 7-aminomethyl-7-deazaguanosine (preQ1), 7-cyano-7-deazaguanosine (preQ0), archaeosine (G+), methylwyosine (mimG), N2,7-dimethylguanosine (m2,7G), N2,N2,2′-O-trimethylguanosine (m22Gm), N2,N2,7-trimethylguanosine (m2,2,7G), N2,N2-dimethylguanosine (m22G), N2,7,2′-O-trimethylguanosine (m2,7Gm), 6-thio-guanosine, 7-deaza-guanosine, 8-oxo-guanosine, N1-methyl-guanosine, a-thio-guanosine, 2(propyl)guanine, 2-(alkyl)guanine, 2′-amino-2′-deoxy-GTP, 2′-azido-2′-deoxy-GTP, 2′-deoxy-2′-a-aminoguanosine TP, 2′-deoxy-2′-a-azidoguanosine TP, 6(methyl)guanine, 6-(alkyl)guanine, 6-(methyl)guanine, 6-methyl-guanosine, 7(alkyl)guanine, 7(deaza)guanine, 7(methyl)guanine, 7-(alkyl)guanine, 7-(deaza)guanine, 7-(methyl)guanine, 8(alkyl)guanine, 8(alkynyl)guanine, 8(halo)guanine, 8(thioalkyl)guanine, 8-(alkenyl)guanine, 8-(alkyl)guanine, 8-(alkynyl)guanine, 8-(amino)guanine, 8-(halo)guanine, 8-(hydroxyl)guanine, 8-(thioalkyl)guanine, 8-(thiol)guanine, aza guanine, deaza guanine, N-(methyl)guanine, 1-methyl-6-thio-guanosine, 6-methoxy-guanosine, 6-thio-7-deaza-8-aza-guanosine, 6-thio-7-deaza-guanosine, 6-thio-7-methyl-guanosine, 7-deaza-8-aza-guanosine, 7-methyl-8-oxo-guanosine, N2,N2-dimethyl-6-thio-guanosine, N2-methyl-6-thio-guanosine, 1-Me-GTP, 2′-fluoro-N2-isobutyl-guanosine TP, 2′-O-methyl-N2-isobutyl-guanosine TP, 2′-a-ethynylguanosine TP, 2′-a-trifluoromethylguanosine TP, 2′-b-ethynylguanosine TP, 2′-b-trifluoromethylguanosine TP, 2′-deoxy-2′,2′-difluoroguanosine TP, 2′-deoxy-2′-a-mercaptoguanosine TP, 2′-deoxy-2′-a-thiomethoxyguanosine TP, 2′-deoxy-2′-b-aminoguanosine TP, 2′-deoxy-2′-b-azidoguanosine TP, 2′-deoxy-2′-b-bromoguanosine TP, 2′-deoxy-2′-b-chloroguanosine TP, 2′-deoxy-2′-b-fluoroguanosine TP, 2′-deoxy-2′-b-iodoguanosine TP, 2′-deoxy-2′-b-mercaptoguanosine TP, 2′-deoxy-2′-b-thiomethoxyguanosine TP, 4′-azidoguanosine TP, 4′-carbocyclic guanosine TP, 4′-ethynylguanosine TP, 5′-homo-guanosine TP, 8-bromo-guanosine TP, 9-deazaguanosine TP, and N2-isobutyl-guanosine TP.

In one embodiment, from about 5% to 100% of the guanosine ribonucleosides within the mRNA are modified. In one embodiment, from about 5%, about 10%, about 15%, about 20%, about 30%, about 40%, about 50%, about 60%, about 70%, about 80%, about 90%, or about 100% of the guanosine ribonucleosides within the mRNA are modified.

In some embodiments, the one or more chemical modifications include modifications to a uridine ribonucleoside within the mRNA. Representative examples of uridine ribonucleoside modifications include, but are not limited to, 2-thiouridine (s2U), 3-methyluridine (m3U), 5-carboxymethyluridine (cm5U), 5-hydroxyuridine (ho5U, 5-methyluridine (m5U), 5-taurinomethyl-2-thiouridine (im5s2U), 5-taurinomethyluridine (im5U), Dihydrouridine (D), Pseudouridine (Ψ), (3-(3-amino-3-carhoxypropyl)uridine (acp3U), 1-methyl-3-(3-amino-5-carboxypropyl)pseudouridine (m1acp3Ψ), 1-methylpseudouridine (m1Ψ), 2′-O-methyluridine (Um), 2′-O-methylpseudouridine (Ψm), 2′-O-methyluridine (Um), 2-thio-2′-O-methyluridine (s2Um), 3-(3-amino-3-carboxypropyl)uridine (acp3U), 3,2′-O-dimethyluridine (m3Um), 3-methyl-pseudo-Uridine TP, 4-thiouridine (s4U), 5-(carboxyhydroxymethyl)uridine (chm5U), 5-(carboxyhydroxymethyl)uridine methyl ester (mchm5U), 5,2′-O-dimethyluridine (m5Um), 5,6-dihydro-uridine, 5-aminomethyl-2-thiouridine (nm5s2U), 5-carbamoylmethyl-2′-O-methyluridine (ncm5Um), 5-carbamoylmethyluridine (ncm5U), 5-carboxyhydroxymethyluridine, 5-carboxyhydroxymethyluridine methyl ester, 5-carboxymethylaminomethyl-2′-O-methyluridine (cmnm5Um), 5-carboxymethylaminomethyl-2-thiouridine (cmnm5s2U), 5-carboxymethylaminomethyl-2-thiouridine, 5-carboxymethylaminomethyluridine (cmnm5U), 5-carboxymethylaminomethyluridine, 5-carbamoylmethyluridine TP, 5-methoxycarbonylmethyl-2′-O-methyluridine (mcm5Um), 5-methoxycarbonylmethyl-2-thiouridine (mcm5s2U), 5-methoxycarbonylmethyluridine (mcm5U), 5-methoxyuridine (mo5U), 5-methyl-2-thiouridine (m5s2U), 5-methylaminomethyl-2-selenouridine (mnm5se2U), 5-methylaminomethyl-2-thiouridine (mnm5s2U), 5-methylaminomethyluridine (mnm5U), 5-methyldihydrouridine, 5-oxyacetic acid-uridine TP, 5-oxyacetic acid-methyl ester-uridine TP, N1-methyl-pseudo-uridine, uridine 5-oxyacetic acid (cmo5U), uridine 5-oxyacetic acid methyl ester (mcmo5U), 3-(3-Amino-3-carboxypropyl)-Uridine TP, 5-(iso-pentenylaminomethyl)-2-thiouridine TP, 5-(iso-pentenylaminomethyl)-2′-O-methyluridine TP, 5-(iso-pentenylaminomethyl)uridine TP, 5-propynyl uracil, a-thio-uridine, 1(aminoalkylamino-carbonylethylenyl)-2(thio)-pseudouracil, 1(aminoalkylaminocarbonylethylenyl)-2,4-(dithio)pseudouracil, 1(aminoalkylaminocarbonylethylenyl)-4(thio)pseudouracil, 1(aminoalkylaminocarbonylethylenyl)-pseudouracil, 1(aminocarbonylethylenyl)-2(thio)-pseudouracil, 1(aminocarbonylethylenyl)-2,4-(dithio)pseudouracil, 1(aminocarbonylethylenyl)-4-(thio)pseudouracil, 1(aminocarbonylethylenyl)-pseudouracil, 1 substituted 2(thio)-pseudouracil, 1 substituted 2,4-(dithio)pseudouracil, 1 substituted 4(thio)pseudouracil, 1 substituted pseudouracil, 1-(aminoalkylamino-carbonylethylenyl)-2-(thio)-pseudouracil, 1-methyl-3-(3-amino-3-carboxypropyl)pseudouridine TP, 1-methyl-3-(3-amino-3-carboxypropyl)pseudo-UTP, 1-methyl-pseudo-UTP, 2 (thio)pseudouracil, 2′-deoxy uridine, 2′-fluorouridine, 2-(thio)uracil, 2,4-(dithio)psuedouracil, 2′-methyl,2′-amino,2′-azido,2′-fluro-guanosine, 2′-Amino-2′-deoxy-UTP, 2′-azido-2′-deoxy-UTP, 2′-azido-deoxyuridine TP, 2′-O-methylpseudouridine, 2′-deoxy uridine, 2′-fluorouridine, 2′-deoxy-2′-a-aminouridine TP, 2′-deoxy-2′-a-azidouridine TP, 2-methylpseudouridine (m3′P), 3(3 amino-3 carboxypropyl)uracil, 4(thio)pseudouracil, 4-(thio)pseudouracil, 4-(thio)uracil, 4-thiouracil, 5(1,3-diazole-1-alkyl)uracil, 5(2-aminopropyl)uracil, 5(aminoalkyl)uracil, 5(dimethylaminoalkyl)uracil, 5(guanidiniumalkyl)uracil, 5(methoxycarbonylmethyl)-2-(thio)uracil, 5(methoxycarbonyl-methyl)uracil, 5(methyl)2(thio)uracil, 5(methyl)2,4(dithio)uracil, 5(methyl)4(thio)uracil, 5(methylaminomethyl)-2(thio)uracil, 5(methylaminomethyl)-2,4(dithio)uracil, 5(methylaminomethyl)-4(thio)uracil, 5(propynyl)uracil, 5(trifluoromethyl)uracil, 5-(2-aminopropyl)uracil, 5-(alkyl)-2-(thio)pseudouracil, 5-(alkyl)-2,4(dithio)pseudouracil, 5-(alkyl)-4(thio)pseudouracil, 5-(alkyl)pseudouracil, 5-(alkyl)uracil, 5-(alkynyl)uracil, 5-(allylamino)uracil, 5-(cyanoalkyl)uracil, 5-(dialkylaminoalkyl)uracil, 5-(dimethylaminoalkyl)uracil, 5-(guanidiniumalkyl)uracil, 5-(halo)uracil, 5-(1,3-diazole-1-alkyl)uracil, 5-(methoxy)uracil, 5-(methoxycarbonylmethyl)-2-(thio)uracil, 5-(methoxycarbonyl-methyl)uracil, 5-(methyl)2(thio)uracil, 5-(methyl)2,4(dithio)uracil, 5-(methyl)4(thio)uracil, 5-(methyl)-2-(thio)pseudouracil, 5-(methyl)-2,4(dithio)pseudouracil, 5-(methyl)-4(thio)pseudouracil, 5-(methyl)pseudouracil, 5-(methylaminomethyl)-2(thio)uracil, 5-(methylaminomethyl)-2,4(dithio)uracil, 5-(methylaminomethyl)-4-(thio)uracil, 5-(propynyl)uracil, 5-(trifluoromethyl)uracil, 5-aminoallyl-uridine, 5-bromo-uridine, 5-iodo-uridine, 5-uracil, 6(azo)uracil, 6-(azo)uracil, 6-aza-uridine, allyamino-uracil, aza uracil, deaza uracil, N3(methyl)uracil, pseudo-UTP-1-2-ethanoic acid, pseudouracil, 4-thio-pseudo-UTP, 1-carboxymethyl-pseudouridine, 1-methyl-1-deaza-pseudouridine, 1-propynyl-uridine, 1-taurinomethyl-1-methyl-uridine, 1-taurinomethyl-4-thio-uridine, 1-taurinomethyl-pseudouridine, 2-methoxy-4-thio-pseudouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, (1)1-(2-hydroxypropyl)pseudouridine TP, (2R)-1-(2-hydroxypropyl)pseudouridine TP, (2S)-1-(2-hydroxypropyl)pseudouridine TP, (E)-5-(2-bromo-vinyl)ara-uridine TP, (E)-5-(2-bromo-vinyl)uridine TP, (Z)-5-(2-bromo-vinyl)ara-uridine TP, (Z)-5-(2-bromo-vinyl)uridine TP, 1-(2,2,2-trifluoroethyl)-pseudo-UTP, 1-(2,2,3,3,3-pentafluoropropyl)pseudouridine TP, 1-(2,2-diethoxyethyl)pseudouridine TP, 1-(2,4,6-trimethylbenzyl)pseudouridine TP, 1-(2,4,6-trimethyl-benzyl)pseudo-UTP, 1-(2,4,6-trimethyl-phenyl)pseudo-UTP, 1-(2-amino-2-carboxyethyl)pseudo-UTP, 1-(2-amino-ethyl)pseudo-UTP, 1-(2-hydroxyethyl)pseudouridine TP, 1-(2-methoxyethyl)pseudouridine TP, 1-(3,4-bis-trifluoromethoxybenzyl)pseudouridine TP, 1-(3,4-dimethoxybenzyl)pseudouridine TP, 1-(3-amino-3-carboxypropyl)pseudo-UTP, 1-(3-amino-propyl)pseudo-UTP, 1-(3-cyclopropyl-prop-2-ynyl)pseudouridine TP, 1-(4-amino-4-carboxybutyl)pseudo-UTP, 1-(4-amino-benzyl)pseudo-UTP, 1-(4-amino-butyl)pseudo-UTP, 1-(4-amino-phenyl)pseudo-UTP, 1-(4-azidobenzyl)pseudouridine TP, 1-(4-bromobenzyl)pseudouridine TP, 1-(4-chlorobenzyl)pseudouridine TP, 1-(4-fluorobenzyl)pseudouridine TP, 1-(4-iodobenzyl)pseudouridine TP, 1-(4-methanesulfonylbenzyl)pseudouridine TP, 1-(4-methoxybenzyl)pseudouridine TP, 1-(4-methoxy-benzyl)pseudo-UTP, 1-(4-methoxy-phenyl)pseudo-UTP, 1-(4-methylbenzyl)pseudouridine TP, 1-(4-methyl-benzyl)pseudo-UTP, 1-(4-nitrobenzyl)pseudouridine TP, 1-(4-nitro-benzyl)pseudo-UTP, 1(4-nitro-phenyl)pseudo-UTP, 1-(4-thiomethoxybenzyl)pseudouridine TP, 1-(4-trifluoromethoxybenzyl)pseudouridine TP, 1-(4-trifluoromethylbenzyl)pseudouridine TP, 1-(5-amino-pentyl)pseudo-UTP, 1-(6-amino-hexyl)pseudo-UTP, 1,6-dimethyl-pseudo-UTP, 1-[3-(2-{2-[2-(2-aminoethoxy)-ethoxy]-ethoxy}-ethoxy)-propionyl]pseudouridine TP, 1-{3-[2-(2-aminoethoxy)-ethoxy]-propionyl}pseudouridine TP, 1-acetylpseudouridine TP, 1-alkyl-6-(1-propynyl)-pseudo-UTP, 1-alkyl-6-(2-propynyl)-pseudo-UTP, 1-alkyl-6-allyl-pseudo-UTP, 1-alkyl-6-ethynyl-pseudo-UTP, 1-alkyl-6-homoallyl-pseudo-UTP, 1-alkyl-6-vinyl-pseudo-UTP, 1-allylpseudouridine TP, 1-aminomethyl-pseudo-UTP, 1-benzoylpseudouridine TP, 1-benzyloxymethylpseudouridine TP, 1-benzyl-pseudo-UTP, 1-biotinyl-PEG2-pseudouridine TP, 1-biotinylpseudouridine TP, 1-butyl-pseudo-UTP, 1-cyanomethylpseudouridine TP, 1-cyclobutylmethyl-pseudo-UTP, 1-cyclobutyl-pseudo-UTP, 1-cycloheptylmethyl-pseudo-UTP, 1-cycloheptyl-pseudo-UTP, 1-cyclohexylmethyl-pseudo-UTP, 1-cyclohexyl-pseudo-UTP, 1-cyclooctylmethyl-pseudo-UTP, 1-cyclooctyl-pseudo-UTP, 1-cyclopentylmethyl-pseudo-UTP, 1-cyclopentyl-pseudo-UTP, 1-cyclopropylmethyl-pseudo-UTP, 1-cyclopropyl-pseudo-UTP, 1-ethyl-pseudo-UTP, 1-hexyl-pseudo-UTP, 1-homoallylpseudouridine TP, 1-hydroxymethylpseudouridine TP, 1-iso-propyl-pseudo-UTP, 1-Me-2-thio-pseudo-UTP, 1-Me-4-thio-pseudo-UTP, 1-Me-alpha-thio-pseudo-UTP, 1-methanesulfonylmethylpseudouridine TP, 1-methoxymethylpseudouridine TP, 1-methyl-6-(2,2,2-Trifluoroethyl)pseudo-UTP, 1-methyl-6-(4-morpholino)-pseudo-UTP, 1-methyl-6-(4-thiomorpholino)-pseudo-UTP, 1-methyl-6-(substituted phenyl)pseudo-UTP, 1-methyl-6-amino-pseudo-UTP, 1-methyl-6-azido-pseudo-UTP, 1-methyl-6-bromo-pseudo-UTP, 1-methyl-6-butyl-pseudo-UTP, 1-methyl-6-chloro-pseudo-UTP, 1-methyl-6-cyano-pseudo-UTP, 1-methyl-6-dimethylamino-pseudo-UTP, 1-methyl-6-ethoxy-pseudo-UTP, 1-methyl-6-ethylcarboxylate-pseudo-UTP, 1-methyl-6-ethyl-pseudo-UTP, 1-methyl-6-fluoro-pseudo-UTP, 1-methyl-6-formyl-pseudo-UTP, 1-methyl-6-hydroxyamino-pseudo-UTP, 1-methyl-6-hydroxy-pseudo-UTP, 1-methyl-6-iodo-pseudo-UTP, 1-methyl-6-iso-propyl-pseudo-UTP, 1-methyl-6-methoxy-pseudo-UTP, 1-methyl-6-methylamino-pseudo-UTP, 1-methyl-6-phenyl-pseudo-UTP, 1-methyl-6-propyl-pseudo-UTP, 1-methyl-6-tert-butyl-pseudo-UTP, 1-methyl-6-trifluoromethoxy-pseudo-UTP, 1-methyl-6-trifluoromethyl-pseudo-UTP, 1-morpholinomethylpseudouridine TP, 1-pentyl-pseudo-UTP, 1-phenyl-pseudo-UTP, 1-pivaloylpseudouridine TP, 1-propargylpseudouridine TP, 1-propyl-pseudo-UTP, 1-propynyl-pseudouridine, 1-p-tolyl-pseudo-UTP, 1-tert-butyl-pseudo-UTP, 1-yhiomethoxymethylpseudouridine TP, 1-yhiomorpholinomethylpseudouridine TP, 1-yrifluoroacetylpseudouridine TP, 1-yrifluoromethyl-pseudo-UTP, 1-vinylpseudouridine TP, 2,2′-anhydro-uridine TP, 2′-bromo-deoxyuridine TP, 2-F-5-Methyl-2′-deoxy-UTP, 2′-OMe-5-Me-UTP, 2-OMe-pseudo-UTP, 2′-a-ethynyluridine TP, 2′-a-trifluoromethyluridine TP, 2′-b-ethynyluridine TP, 2′-b-trifluoromethyluridine TP, 2′-deoxy-2′,2′-difluorouridine TP, 2′-deoxy-2′-a-mercaptouridine TP, 2′-deoxy-2′-a-thiomethoxyuridine TP, 2′-deoxy-2′-b-aminouridine TP, 2′-deoxy-2′-b-azidouridine TP, 2′-deoxy-2′-b-bromouridine TP, 2′-deoxy-2′-b-chlorouridine TP, 2′-deoxy-2′-b-fluorouridine TP, 2′-deoxy-2′-b-iodouridine TP, 2′-deoxy-2′-b-mercaptouridine TP, 2′-deoxy-2′-b-thiomethoxyuridine TP, 2-methoxy-4-thio-uridine, 2-methoxyuridine, 2′-O-methyl-5-(1-propynyl)uridine TP, 3-alkyl-pseudo-UTP, 4′-azidouridine TP, 4′-carbocyclic uridine TP, 4′-ethynyluridine TP, 5-(1-propynyl)ara-uridine TP, 5-(2-furanyl)uridine TP, 5-cyanouridine TP, 5-dimethylaminouridine TP, 5′-homo-uridine TP, 5-iodo-2′-fluoro-deoxyuridine TP, 5-phenylethynyluridine TP, 5-trideuteromethyl-6-deuterouridine TP, 5-trifluoromethyl-Uridine TP, 5-vinylarauridine TP, 6-(2,2,2-trifluoroethyl)-pseudo-UTP, 6-(4-morpholino)-pseudo-UTP, 6-(4-thiomorpholino)-pseudo-UTP, 6-(substituted-phenyl)-pseudo-UTP, 6-amino-pseudo-UTP, 6-azido-pseudo-UTP, 6-bromo-pseudo-UTP, 6-butyl-pseudo-UTP, 6-chloro-pseudo-UTP, 6-cyano-pseudo-UTP, 6-dimethylamino-pseudo-UTP, 6-ethoxy-pseudo-UTP, 6-ethylcarboxylate-pseudo-UTP, 6-ethyl-pseudo-UTP, 6-fluoro-pseudo-UTP, 6-formyl-pseudo-UTP, 6-hydroxyamino-pseudo-UTP, 6-hydroxy-pseudo-UTP, 6-iodo-pseudo-UTP, 6-iso-propyl-pseudo-UTP, 6-methoxy-pseudo-UTP, 6-methylamino-pseudo-UTP, 6-methyl-pseudo-UTP, 6-phenyl-pseudo-UTP, 6-propyl-pseudo-UTP, 6-tert-butyl-pseudo-UTP, 6-trifluoromethoxy-pseudo-UTP, 6-trifluoromethyl-pseudo-UTP, alpha-thio-pseudo-UTP, pseudouridine 1-(4-methylhenzenesulfonic acid) TP, pseudouridine 1-(4-methylbenzoic acid) TP, pseudouridine TP 1-[3-(2-ethoxy)]propionic acid, pseudouridine TP 1-[3-{2-(2-[2-(2-ethoxy)-ethoxy]-ethoxy)-ethoxy}]propionic acid, pseudouridine TP 1-[3-{2-(2-[2-{2(2-ethoxy)-ethoxy}-ethoxy]-ethoxy)-ethoxy}]propionic acid, pseudouridine TP 1-[3-{2-(2-[2-ethoxy]-ethoxy)-ethoxy}]propionic acid. pseudouridine TP 1-[3-{2-(2-ethoxy)-ethoxy}] propionic acid, pseudouridine TP 1-methylphosphonic acid, pseudouridine TP 1-methylphosphonic acid diethyl ester, pseudo-UTP-N1-3-propionic acid, pseudo-UTP-N1-4-butanoic acid, pseudo-UTP-N1-5-peritanoic acid, pseudo-UTP-N1-6-hexanoic acid, pseudo-UTP-N1-7-heptanoic acid, pseudo-UTP-N1-methyl-p-benzoic acid, and pseudo-UTP-N1-p-benzoic acid.

In one embodiment, from about 5% to 100% of the uridine ribonucleosides within the mRNA are modified. In one embodiment, from about 5%, about 10%, about 15%, about 20%, about 30%, about 40%, about 50%, about 60%, about 70%, about 80%, about 90%, or about 100% of the uridine ribonucleosides within the mRNA are modified.

### 5′ Untranslated Regions (5′ UTR) and 3′ Untranslated Regions (3′ UTR)

The mRNA of the present invention may comprise one or more regions or parts, which act or function as an untranslated region. By definition, wild type untranslated regions (UTRs) of a gene are transcribed but not translated. In mRNA, the 5′ UTR starts at the transcription start site and continues to the start codon but does not include the start codon. The 3′ UTR starts immediately following the stop codon and continues until the transcriptional termination signal. There is increasing evidence that UTRs play a regulatory role in terms of stability of the polynucleotide and translation. The regulatory features of a UTR can be incorporated into the mRNA of the present invention to enhance the stability of the molecule, for example. Specific features can also be incorporated to ensure controlled down-regulation of the transcription when present in undesired organ sites, for example miRNA binding sites and RNA binding protein (RBP) binding sites.

Specific 5′ UTR and 3′UTR for use in the present invention can be any suitable UTR sequence, for example, a natural UTR sequence, a derivatized naturally occurring UTR, or a synthetic UTR. In some embodiments, the 5′ UTR and/or the 3′ UTR is a naturally occurring human UTR or a human-derived UTR. The use of human-derived UTRs may facilitate the expression of the polypeptide encoded by the coding region in human cells. In other embodiments, the 5′ UTR and/or the 3′ UTR are synthetic, i.e. not completely homologous with a UTR found in any species. The 5′ UTR is operably linked to the 5′ end of the coding region. The 3′ UTR is operably linked to the 3′ end of the coding region.

Natural 5′ UTRs have features which play roles in translation initiation. They can harbor, for example, Kozak consensus sequences which are known to be involved in the process by which the ribosome initiates translation. The Kozak consensus has the sequence GCCNCCAUGG (SEQ. ID. NO.: 5), where N is a purine (adenine or guanine) three nucleobases upstream from the start codon AUG. 5′ UTRs have also been known to form secondary structures which are involved in elongation factor binding.

By using the polynucleotide sequence features found in abundantly expressed genes of target organs, one can enhance the stability and protein production of the mRNA. For example, incorporating a 5′ UTR sequence from liver-expressed mRNA, for example such as albumin, serum amyloid A, Apolipoprotein A/B/E, transferrin, alpha fetoprotein, erythropoietin, or Factor VIII, into the mRNA of the present invention could be used to enhance its expression within hepatic cell. Likewise, use of a 5′ UTR from other tissue-specific mRNA to improve expression in that tissue is possible for muscle (for example, but not limited to, MyoD, Myosin, Myoglobin, Myogenin, Herculin), for endothelial cells (for example, but not limited to, Tie-1, CD36), for myeloid cells (for example, but not limited to, C/EBP, AML1, G-CSF, GM-CSF, CD11b, MSR, Fr-1, i-NOS), for leukocytes (for example, but not limited to, CD45, CD18), for adipose tissue (for example, but not limited to, CD36, GLUT4, ACRP30, adiponectin), and for lung epithelial cells (for example, but not limited to, SP-A/B/C/D). Untranslated regions useful in the design and manufacture of mRNA include, but are not limited to, those disclosed in International Application Publication No. WO2014164253, incorporated herein by reference in its entirety.

Other non-UTR sequences may be also used as regions or subregions within the mRNA. Combinations of features may be included in regions flanking the coding region and may be contained within other features. For example, the coding region may be flanked by a 5′ UTR which may contain a strong Kozak consensus sequence. WO2014164253 provides a list of exemplary UTRs which may be used as flanking regions and is incorporated herein by reference. Variants of 5′ or 3′ UTRs may be used wherein one or more nucleotides are added or removed at the termini.

Any UTR may be incorporated into the mRNA. For example, multiple wild-type UTRs may be used. Alternatively, UTRs derivatized from a wild-type UTR may be used. In addition, artificial UTRs may be used that are not variants of wild-type regions. These UTRs or portions thereof may be placed in the same orientation as the transcript from which they were selected or may be altered in orientation or location. A 5′ or 3′ UTR may be shortened, lengthened, or made from one or more other 5′ or 3′ UTRs. A UTR may be “altered”, meaning that the UTR has been changed in some way relative to the reference sequence. For example, a 5′ or 3′ UTR may be altered relative to the native UTR by a change in orientation or location, by the inclusion of additional nucleotides, deletion of nucleotides, or by swapping or transposing nucleotides.

In one embodiment, a double, triple, or quadruple UTR such as a 5′ or 3′ UTR may be used. A double UTR is one in which two copies of the UTR are encoded in series or substantially in series. In another embodiment, a patterned 3′ or 5′ UTR may be used. A patterned UTR are those which reflect a repeating or alternative pattern, such as ABABAB or AABBAABBAABB or ABCABCABC or variants thereof repeated once, twice, or more than three times. In these patterns, each letter A, B, or C represents a different UTR at the nucleotide level.

In one embodiment, flanking regions are selected from a family of transcripts whose proteins share a common function, structure, feature, or property. For example, the Oas1b protein belongs to the 2′-5′ oligoadenylate synthetase family of proteins. The UTRs of any of these genes may be swapped for any other UTRs of the same or different family of proteins to create a new mRNA.

The untranslated region may also include translation enhancer elements (TEEs). As a non-limiting example, the TEE may include those described in U.S. Patent Publication No. 20090226470, which is incorporated herein by reference.

Native 3′ UTRs are known to have stretches of adenosines and uridines embedded in them. These AU rich signatures are particularly prevalent in genes with high rates of turnover. Based on their sequence features and functional properties, these AU rich elements (AREs) can be separated into three classes. Class I AREs contain several dispersed copies of an AUUUA motif within uridine-rich regions. Class II AREs contain two or more UUAUUUA(U/A)(U/A) nonamers. Class III AREs are less well defined; these uridine-rich regions do not contain an AUUUA motif. Most proteins binding to AREs are known to destabilize mRNA, wherein members of the ELAV family, most notably HuR, have been documented to increase the stability of mRNA. HuR binds to AREs of all the three classes. Engineering the HuR specific binding sites into the 3′ UTR of the mRNA of the present invention will lead to HuR binding and subsequent stabilization of the mRNA in vivo.

Introduction, removal, or modification of 3′ UTR AU rich elements (AREs) can be used to modify the stability of the mRNA of the present invention. One or more copies of an ARE can be introduced into the 3′UTR of the mRNA to make it less stable, leading to lowered translation and decreased production of the resultant protein. Alternatively, AREs can be identified and removed or mutated to increase the intracellular stability, increasing translation and production of the resultant protein.

MicroRNAs (or miRNA) are 19-25 nucleotide long noncoding RNAs that bind to the 3′ UTR of nucleic acid molecules and down-regulate gene expression either by reducing nucleic acid molecule stability or by inhibiting translation. The mRNA of the invention may comprise one or more microRNA target or binding sequences. microRNA target or binding sequences are well known in the art.

A microRNA sequence comprises a “seed” region, i.e., a sequence in the region of positions 2-8 of the mature microRNA, which sequence has perfect Watson-Crick complementarity to the miRNA target sequence. A microRNA seed may comprise positions 2-8 or 2-7 of the mature microRNA. In some embodiments, a microRNA seed may comprise 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed may comprise 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K, Johnston W K, Garrett-Engele P, Lim L P, Bartel D P; Mol Cell. 2007 Jul. 6; 27(1):91-105, which is incorporated herein by reference. The bases of the microRNA seed have complete complementarity with the target sequence. By engineering microRNA target sequences into the polynucleotides (e.g., in a 3′UTR like region or other region) of the invention one can target the molecule for degradation or reduced translation, provided the microRNA in question is available. This process will reduce the hazard of off target effects upon nucleic acid molecule delivery. Identification of microRNA, microRNA target regions, and their expression patterns and role in biology have been reported (Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414, which are incorporated herein by reference).

For example, if the nucleic acid molecule is an mRNA and is not intended to be delivered to the liver but ends up there, then miR-122, a microRNA abundant in liver, can inhibit the expression of the gene of interest if one or multiple target sites of miR-122 are engineered into the 3′UTR region of the polynucleotides. Introduction of one or multiple binding sites for different microRNA can be engineered to further decrease the longevity, stability, and protein translation of polynucleotides.

As used herein, the term “microRNA target sequence” or “microRNA binding sequence” refers to a microRNA target site or a microRNA recognition site, or any nucleotide sequence to which a microRNA binds or associates. It should be understood that “binding” may follow traditional Watson-Crick hybridization rules or may reflect any stable association of the microRNA with the target sequence at or adjacent to the microRNA site.

Conversely, for the purposes of the mRNA of the present invention, microRNA binding sites can be engineered out of (i.e. removed from) sequences in which they occur, e.g., in order to increase protein expression in specific tissues. For example, miR-122 binding sites may be removed to improve protein expression in the liver. Regulation of expression in multiple tissues can be accomplished through introduction or removal or one or several microRNA binding sites.

Examples of tissues where microRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126). MicroRNA can also regulate complex biological processes such as angiogenesis (miR-132) (Anand and Cheresh Curr Opin Hematol 2011 18:171-176; herein incorporated by reference in its entirety).

In the mRNA of the present invention, binding sites for microRNAs that are involved in such processes may be removed or introduced, in order to tailor the expression of the polynucleotides to biologically relevant cell types or to the context of relevant biological processes.

Examples of use of microRNA to drive tissue or disease-specific gene expression are described in, for example Getner and Naldini, Tissue Antigens. 2012, 80:393-403, herein incorporated by reference in its entirety. In addition, microRNA binding sequences can be incorporated into mRNA to decrease expression in certain cells which results in a biological improvement.

Lastly, through an understanding of the expression patterns of microRNA in different cell types, mRNA can be engineered for more targeted expression in specific cell types or only under specific biological conditions. Through introduction of tissue-specific microRNA binding sites, polynucleotides can be designed that would be optimal for protein expression in a tissue or in the context of a biological condition.

Table 2 below provides exemplary 5′ UTRs that may be used in the mRNA s of the present invention. Variations of these 5′ UTRs may be used wherein one or more nucleotides are added or removed to the termini, including A, U, C, or G.

TABLE 2
Exemplary 5′ Untranslated Regions

| SEQ. | |
|---|---|
| ID. | |
| NO. | Sequence |
| 6 | GGAAAUAAGAGAGAAAAGAAGAGUAAGAAGAAAUAUAAGAGCC |
| | ACC |
| 7 | GGGAAAUAAGAGAGAAAAGAAGAGUAAGAAGAAAUAUAAGAGC |
| | CACC |
| 8 | GGGAGAUCAGAGAGAAAAGAAGAGUAAGAAGAAAUAUAAGAGC |
| | CACC |
| 9 | GGAAUAAAAGUCUCAACACAACAUAUACAAAACAAACGAAUCUC |
| | AAGCAAUCAAGCAUUCUACUUCUAUUGCAGCAAUUUAAAUCAUU |
| | UCUUUUAAAGCAAAAGCAAUUUUCUGAAAAUUUUCACCAUUUA |
| | CGAACGAUAGCAAC |
| 10 | GGGAGACAAGCUUGGCAUUCCGGUACUGUUGGUAAAGCCACC |
| 11 | UCAAGCUUUUGGACCCUCGUACAGAAGCUAAUACGACUCACUAU |
| | AGGGAAAUAAGAGAGAAAAGAAGAGUAAGAAGAAAUAUAAGAG |
| | CCACC |
| 12 | AUGGCCGGACCCGCGACUCAGAGCCCAAUGAAGCUGAUGGCUCU |
| | UCA |
| 13 | GGGAAAUAAGAGAGAAAAGAAGAGUAAGAAGAAAUAUAAGAGC |
| | CACC |
| 14 | GGGAAUUAACAGAGAAAAGAAGAGUAAGAAGAAAUAUAAGAGC |
| | CACC |
| 15 | GGGAAAUUAGACAGAAAAGAAGAGUAAGAAGAAAUAUAAGAGC |
| | CACC |
| 16 | GGGAAAUAAGAGAGUAAAGAACAGUAAGAAGAAAUAUAAGAGC |
| | CACC |
| 17 | GGGAAAAAAGAGAGAAAAGAAGACUAAGAAGAAAUAUAAGAGC |
| | CACC |
| 18 | GGGAAAUAAGAGAGAAAAGAAGAGUAAGAAGAUAUAUAAGAGC |
| | CACC |
| 19 | GGGAAAUAAGAGACAAAACAAGAGUAAGAAGAAAUAUAAGAGC |
| | CACC |
| 20 | GGGAAAUUAGAGAGUAAAGAACAGUAAGUAGAAUUAAAAGAGC |
| | CACC |
| 21 | GGGAAAUAAGAGAGAAUAGAAGAGUAAGAAGAAAUAUAAGAGC |
| | CACC |
| 22 | GGGAAAUAAGAGAGAAAAGAAGAGUAAGAAGAAAAUUAAGAGC |
| | CACC |
| 23 | GGGAAAUAAGAGAGAAAAGAAGAGUAAGAAGAAAUUUAAGAGC |
| | CACC |

Table 3 below provides exemplary 3′UTRs that may be used in the mRNA of the present invention. Variations of these 3′UTRs may be used wherein one or more nucleotides are added or removed to the termini, including A, U, C, or G.

TABLE 3
Exemplary 3′ Untranslated Regions

| SEQ. | |
|---|---|
| ID. | |
| NO. | Sequence |
| 24 | GCUGCCUUCUGCGGGGCUUGCCUUCUGGCCAUGCCCUUCUUCUC |
| | UCCCUUGCACCUGUACCUCUUGGUCUUUGAAUAAAGCCUGAGUA |
| | GGAAGGCGGCCGCAAAAA |
| 25 | GCGCCUGCCCACCUGCCACCGACUGCUGGAACCCAGCCAGUGGG |
| | AGGGCCUGGCCCACCAGAGUCCUGCUCCCUCACUCCUCGCCCCGC |
| | CCCCUGUCCCAGAGUCCCACCUGGGGGCUCUCUCCACCCUUCUCA |
| | GAGUUCCAGUUUCAACCAGAGUUCCAACCAAUGGGCUCCAUCCU |
| | CUGGAUUCUGGCCAAUGAAAUAUCUCCCUGGCAGGGUCCUCUUC |
| | UUUUCCCAGAGCUCCACCCCAACCAGGAGCUCUAGUUAAUGGAG |
| | AGCUCCCAGCACACUCGGAGCUUGUGCUUUGUCUCCACGCAAAG |
| | CGAUAAAUAAAAGCAUUGGUGGCCUUUGGUCUUUGAAUAAAGC |
| | CUGAGUAGGAAGUCUAGA |
| 26 | GCCCCUGCCGCUCCCACCCCCACCCAUCUGGGCCCCGGGUUCAAG |
| | AGAGAGCGGGGUCUGAUCUCGUGUAGCCAUAUAGAGUUUGCUU |
| | CUGAGUGUCUGCUUUGUUUAGUAGAGGUGGGCAGGAGGAGCUG |
| | AGGGGCUGGGGCUGGGGUGUUGAAGUUGGCUUUGCAUGCCCAG |
| | CGAUGCGCCUCCCUGUGGGAUGUCAUCACCCUGGGAACCGGGAG |
| | UGGCCCUUGGCUCACUGUGUUCUGCAUGGUUUGGAUCUGAAUUA |
| | AUUGUCCUUUCUUCUAAAUCCCAACCGAACUUCUUCCAACCUCC |
| | AAACUGGCUGUAACCCCAAAUCCAAGCCAUUAACUACACCUGAC |
| | AGUAGCAAUUGUCUGAUUAAUCACUGGCCCCUUGAAGACAGCAG |
| | AAUGUCCCUUUGCAAUGAGGAGGAGAUCUGGGCUGGGCGGGCCA |
| | GCUGGGGAAGCAUUUGACUAUCUGGAACUUGUGUGUGCCUCCUC |
| | AGGUAUGGCAGUGACUCACCUGGUUUUAAUAAAACAACCUGCAA |
| | CAUCUCAUGGUCUUUGAAUAAAGCCUGAGUAGGAAGUCUAGA |
| 27 | ACACACUCCACCUCCAGCACGCGACUUCUCAGGACGACGAAUCU |
| | UCUCAAUGGGGGGGCGGCUGAGCUCCAGCCACCCCGCAGUCACU |
| | UUCUUUGUAACAACUUCCGUUGCUGCCAUCGUAAACUGACACAG |
| | UGUUUAUAACGUGUACAUACAUUAACUUAUUACCUCAUUUUGU |
| | UAUUUUUCGAAACAAAGCCCUGUGGAAGAAAAUGGAAAACUUG |
| | AAGAAGCAUUAAAGUCAUUCUGUUAAGCUGCGUAAAUGGUCUU |
| | UGAAUAAAGCCUGAGUAGGAAGUCUAGA |
| 28 | CAUCACAUUUAAAAGCAUCUCAGCCUACCAUGAGAAUAAGAGAA |
| | AGAAAAUGAAGAUCAAAAGCUUAUUCAUCUGUUUUUCUUUUUC |
| | GUUGGUGUAAAGCCAACACCCUGUCUAAAAAACAUAAAUUUCUU |
| | UAAUCAUUUUGCCUCUUUUCUCUGUGCUUCAAUUAAUAAAAAA |
| | UGGAAAGAAUCUAAUAGAGUGGUACAGCACUGUUAUUUUUCAA |
| | AGAUGUGUUGCUAUCCUGAAAAUUCUGUAGGUUCUGUGGAAGU |
| | UCCAGUGUUCUCUCUUAUUCCACUUCGGUAGAGGAUUUCUAGUU |
| | UCUUGUGGGCUAAUUAAAUAAAUCAUUAAUACUCUUCUAAUGG |
| | UCUUUGAAUAAAGCCUGAGUAGGAAGUCUAGA |
| 29 | GCUGCCUUCUGCGGGGCUUGCCUUCUGGCCAUGCCCUUCUUCUC |
| | UCCCUUGCACCUGUACCUCUUGGUCUUUGAAUAAAGCCUGAGUA |
| | GGAAGGCGGCCGCUCGAGCAUGCAUCUAGA |
| 30 | GCCAAGCCCUCCCCAUCCCAUGUAUUUAUCUCUAUUUAAUAUUU |
| | AUGUCUAUUUAAGCCUCAUAUUUAAAGACAGGGAAGAGCAGAA |
| | CGGAGCCCCAGGCCUCUGUGUCCUUCCCUGCAUUUCUGAGUUUC |
| | AUUCUCCUGCCUGUAGCAGUGAGAAAAAGCUCCUGUCCUCCCAU |
| | CCCCUGGACUGGGAGGUAGAUAGGUAAAUACCAAGUAUUUAUU |
| | ACUAUGACUGCUCCCCAGCCCUGGCUCUGCAAUGGGCACUGGGA |
| | UGAGCCGCUGUGAGCCCCUGGUCCUGAGGGUCCCCACCUGGGAC |
| | CCUUGAGAGUAUCAGGUCUCCCACGUGGGAGACAAGAAAUCCCU |
| | GUUUAAUAUUUAAACAGCAGUGUUCCCCAUCUGGGUCCUUGCAC |
| | CCCUCACUCUGGCCUCAGCCGACUGCACAGCGGCCCCUGCAUCCC |
| | CUUGGCUGUGAGGCCCCUGGACAAGCAGAGGUGGCCAGAGCUGG |
| | GAGGCAUGGCCCUGGGGUCCCACGAAUUUGCUGGGGAAUCUCGU |
| | UUUUCUUCUUAAGACUUUUGGGACAUGGUUUGACUCCCGAACAU |
| | CACCGACGCGUCUCCUGUUUUUCUGGGUGGCCUCGGGACACCUG |
| | CCCUGCCCCCACGAGGGUCAGGACUGUGACUCUUUUUAGGGCCA |
| | GGCAGGUGCCUGGACAUUUGCCUUGCUGGACGGGGACUGGGGAU |
| | GUGGGAGGGAGCAGACAGGAGGAAUCAUGUCAGGCCUGUGUGU |
| | GAAAGGAAGCUCCACUGUCACCCUCCACCUCUUCACCCCCCACUC |
| | ACCAGUGUCCCCUCCACUGUCACAUUGUAACUGAACUUCAGGAU |
| | AAUAAAGUGUUUGCCUCCAUGGUCUUUGAAUAAAGCCUGAGUA |
| | GGAAGGCGGCCGCUCGAGCAUGCAUCUAGA |
| 31 | ACUCAAUCUAAAUUAAAAAAGAAAGAAAUUUGAAAAAACUUUC |
| | UCUUUGCCAUUUCUUCUUCUUCUUUUUUAACUGAAAGCUGAAUC |
| | CUUCCAUUUCUUCUGCACAUCUACUUGCUUAAAUUGUGGGCAAA |
| | AGAGAAAAAGAAGGAUUGAUCAGAGCAUUGUGCAAUACAGUUU |
| | CAUUAACUCCUUCCCCCGCUCCCCCAAAAAUUUGAAUUUUUUUU |
| | UCAACACUCUUACACCUGUUAUGGAAAAUGUCAACCUUUGUAAG |
| | AAAACCAAAAUAAAAAUUGAAAAAUAAAAACCAUAAACAUUUG |
| | CACCACUUGUGGCUUUUGAAUAUCUUCCACAGAGGGAAGUUUAA |
| | AACCCAAACUUCCAAAGGUUUAAACUACCUCAAAACACUUUCCC |
| | AUGAGUGUGAUCCACAUUGUUAGGUGCUGACCUAGACAGAGAU |
| | GAACUGAGGUCCUUGUUUUGUUUUGUUCAUAAUACAAAGGUGC |
| | UAAUUAAUAGUAUUUCAGAUACUUGAAGAAUGUUGAUGGUGCU |
| | AGAAGAAUUUGAGAAGAAAUACUCCUGUAUUGAGUUGUAUCGU |
| | GUGGUGUAUUUUUUAAAAAAUUUGAUUUAGCAUUCAUAUUUUC |
| | CAUCUUAUUCCCAAUUAAAAGUAUGCAGAUUAUUUGCCCAAAUC |
| | UUCUUCAGAUUCAGCAUUUGUUCUUUGCCAGUCUCAUUUUCAUC |
| | UUCUUCCAUGGUUCCACAGAAGCUUUGUUUCUUGGGCAAGCAGA |
| | AAAAUUAAAUUGUACCUAUUUUGUAUAUGUGAGAUGUUUAAAU |
| | AAAUUGUGAAAAAAAUGAAAUAAAGCAUGUUUGGUUUUCCAAA |
| | AGAACAUAU |
| 32 | CGCCGCCGCCCGGGCCCCGCAGUCGAGGGUCGUGAGCCCACCCCG |
| | UCCAUGGUGCUAAGCGGGCCCGGGUCCCACACGGCCAGCACCGC |
| | UGCUCACUCGGACGACGCCCUGGGCCUGCACCUCUCCAGCUCCU |
| | CCCACGGGGUCCCCGUAGCCCCGGCCCCCGCCCAGCCCCAGGUCU |
| | CCCCAGGCCCUCCGCAGGCUGCCCGGCCUCCCUCCCCCUGCAGCC |
| | AUCCCAAGGCUCCUGACCUACCUGGCCCCUGAGCUCUGGAGCAA |
| | GCCCUGACCCAAUAAAGGCUUUGAACCCAU |
| 33 | GGGGCUAGAGCCCUCUCCGCACAGCGUGGAGACGGGGCAAGGAG |
| | GGGGGUUAUUAGGAUUGGUGGUUUUGUUUUGCUUUGUUUAAAG |
| | CCGUGGGAAAAUGGCACAACUUUACCUCUGUGGGAGAUGCAACA |
| | CUGAGAGCCAAGGGGUGGGAGUUGGGAUAAUUUUUAUAUAAAA |
| | GAAGUUUUUCCACUUUGAAUUGCUAAAAGUGGCAUUUUUCCUA |
| | UGUGCAGUCACUCCUCUCAUUUCUAAAAUAGGGACGUGGCCAGG |
| | CACGGUGGCUCAUGCCUGUAAUCCCAGCACUUUGGGAGGCCGAG |
| | GCAGGCGGCUCACGAGGUCAGGAGAUCGAGACUAUCCUGGCUAA |
| | CACGGUAAAACCCUGUCUCUACUAAAAGUACAAAAAAUUAGCUG |
| | GGCGUGGUGGUGGGCACCUGUAGUCCCAGCUACUCGGGAGGCUG |
| | AGGCAGGAGAAAGGCAUGAAUCCAAGAGGCAGAGCUUGCAGUG |
| | AGCUGAGAUCACGCCAUUGCACUCCAGCCUGGGCAACAGUGUUA |
| | AGACUCUGUCUCAAAUAUAAAUAAAUAAAUAAAUAAAUAAAUA |
| | AAUAAAUAAAAAUAAAGCGAGAUGUUGCCCUCAAA |
| 34 | UGAUAAUAGGCUGGAGCCUCGGUGGCCAUGCUUCUUGCCCCUUG |
| | GGCCUCCCCCCAGCCCCUCCUCCCCUUCCUGCACCCGUACCCCCG |
| | UGGUCUUUGAAUAAAGUCUGAGUGGGCGGC |
| 35 | AUAUUAAGGAUCAAGCUGUUAGCUAAUAAUGCCACCUCUGCAGU |
| | UUUGGGAACAGGCAAAUAAAGUAUCAGUAUACAUGGUGAUGUA |
| | CAUCUGUAGCAAAGCUCUUGGAGAAAAUGAAGACUGAAGAAAG |
| | CAAAGCAAAAACUGUAUAGAGAGAUUUUUCAAAAGCAGUAAUC |
| | CCUCAAUUUUAAAAAAGGAUUGAAAAUUCUAAAUGUCUUUCUG |
| | UGCAUAUUUUUUGUGUUAGGAAUCAAAAGUAUUUUAUAAAAGG |
| | AGAAAGAACAGCCUCAUUUUAGAUGUAGUCCUGUUGGAUUUUU |
| | UAUGCCUCCUCAGUAACCAGAAAUGUUUUAAAAAACUAAGUGU |
| | UUAGGAUUUCAAGACAACAUUAUACAUGGCUCUGAAAUAUCUG |
| | ACACAAUGUAAACAUUGCAGGCACCUGCAUUUUAUGUUUUUUU |
| | UUUCAACAAAUGUGACUAAUUUGAAACUUUUAUGAACUUCUGA |
| | GCUGUCCCCUUGCAAUUCAACCGCAGUUUGAAUUAAUCAUAUCA |
| | AAUCAGUUUUAAUUUUUUAAAUUGUACUUCAGAGUCUAUAUUU |
| | CAAGGGCACAUUUUCUCACUACUAUUUUAAUACAUUAAAGGACU |
| | AAAUAAUCUUUCAGAGAUGCUGGAAACAAAUCAUUUGCUUUAU |
| | AUGUUUCAUUAGAAUACCAAUGAAACAUACAACUUGAAAAUUA |
| | GUAAUAGUAUUUUUGAAGAUCCCAUUUCUAAUUGGAGAUCUCU |
| | UUAAUUUCGAUCAACUUAUAAUGUGUAGUACUAUAUUAAGUGC |
| | ACUUGAGUGGAAUUCAACAUUUGACUAAUAAAAUGAGUUCAUC |
| | AUGUUGGCAAGUGAUGUGGCAAUUAUCUCUGGUGACAAAAGAG |
| | UAAAAUCAAAUAUUUCUGCCUGUUACAAAUAUCAAGGAAGACC |
| | UGCUACUAUGAAAUAGAUGACAUUAAUCUGUCUUCACUGUUUA |
| | UAAUACGGAUGGAUUUUUUUUCAAAUCAGUGUGUGUUUUGAGG |
| | UCUUAUGUAAUUGAUGACAUUUGAGAGAAAUGGUGGCUUUUUU |
| | UAGCUACCUCUUUGUUCAUUUAAGCACCAGUAAAGAUCAUGUCU |
| | UUUUAUAGAAGUGUAGAUUUUCUUUGUGACUUUGCUAUCGUGC |
| | CUAAAGCUCUAAAUAUAGGUGAAUGUGUGAUGAAUACUCAGAU |
| | UAUUUGUCUCUCUAUAUAAUUAGUUUGGUACUAAGUUUCUCAA |
| | AAAAUUAUUAACACAUGAAAGACAAUCUCUAAACCAGAAAAAG |
| | AAGUAGUACAAAUUUUGUUACUGUAAUGCUCGCGUUUAGUGAG |
| | UUUAAAACACACAGUAUCUUUUGGUUUUAUAAUCAGUUUCUAU |
| | UUUGCUGUGCCUGAGAUUAAGAUCUGUGUAUGUGUGUGUGUGU |
| | GUGUGUGCGUUUGUGUGUUAAAGCAGAAAAGACUUUUUUAAAA |
| | GUUUUAAGUGAUAAAUGCAAUUUGUUAAUUGAUCUUAGAUCAC |
| | UAGUAAACUCAGGGCUGAAUUAUACCAUGUAUAUUCUAUUAGA |
| | AGAAAGUAAACACCAUCUUUAUUCCUGCCCUUUUUCUUCUCUCA |
| | AAGUAGUUGUAGUUAUAUCUAGAAAGAAGCAAUUUUGAUUUCU |
| | UGAAAAGGUAGUUCCUGCACUCAGUUUAAACUAAAAAUAAUCA |
| | UACUUGGAUUUUAUUUAUUUUUGUCAUAGUAAAAAUUUUAAUU |
| | UAUAUAUAUUUUUAUUUAGUAUUAUCUUAUUCUUUGCUAUUUG |
| | CCAAUCCUUUGUCAUCAAUUGUGUUAAAUGAAUUGAAAAUUCA |
| | UGCCCUGUUCAUUUUAUUUUACUUUAUUGGUUAGGAUAUUUAA |
| | AGGAUUUUUGUAUAUAUAAUUUCUUAAAUUAAUAUUCCAAAAG |
| | GUUAGUGGACUUAGAUUAUAAAUUAUGGCAAAAAUCUAAAAAC |
| | AACAAAAAUGAUUUUUAUACAUUCUAUUUCAUUAUUCCUCUUU |
| | UUCCAAUAAGUCAUACAAUUGGUAGAUAUGACUUAUUUUAUUU |
| | UUGUAUUAUUCACUAUAUCUUUAUGAUAUUUAAGUAUAAAUAA |
| | UUAAAAAAAUUUAUUGUACCUUAUAGUCUGUCACCAAAAAAAA |
| | AAAAUUAUCUGUAGGUAGUGAAAUGCUAAUGUUGAUUUGUCUU |
| | UAAGGGCUUGUUAACUAUCCUUUAUUUUCUCAUUUGUCUUAAA |
| | UUAGGAGUUUGUGUUUAAAUUACUCAUCUAAGCAAAAAAUGUA |
| | UAUAAAUCCCAUUACUGGGUAUAUACCCAAAGGAUUAUAAAUC |
| | AUGCUGCUAUAAAGACACAUGCACACGUAUGUUUAUUGCAGCAC |
| | UAUUCACAAUAGCAAAGACUUGGAACCAACCCAAAUGUCCAUCA |
| | AUGAUAGACUUGAUUAAGAAAAUGUGCACAUAUACACCAUGGA |
| | AUACUAUGCAGCCAUAAAAAAGGAUGAGUUCAUGUCCUUUGUA |
| | GGGACAUGGAUAAAGCUGGAAACCAUCAUUCUGAGCAAACUAU |
| | UGCAAGGACAGAAAACCAAACACUGCAUGUUCUCACUCAUAGGU |
| | GGGAAUUGAACAAUGAGAACACUUGGACACAAGGUGGGGAACA |
| | CCACACACCAGGGCCUGUCAUGGGGUGGGGGGAGUGGGGAGGGA |
| | UAGCAUUAGGAGAUAUACCUAAUGUAAAUGAUGAGUUAAUGGG |
| | UGCAGCACACCAACAUGGCACAUGUAUACAUAUGUAGCAAACCU |
| | GCACGUUGUGCACAUGUACCCUAGAACUUAAAGUAUAAUUAAA |
| | AAAAAAAAGAAAACAGAAGCUAUUUAUAAAGAAGUUAUUUGCU |
| | GAAAUAAAUGUGAUCUUUCCCAUUAAAAAAAUAAAGAAAUUUU |
| | GGGGUAAAAAAACACAAUAUAUUGUAUUCUUGAAAAAUUCUAA |
| | GAGAGUGGAUGUGAAGUGUUCUCACCACAAAAGUGAUAACUAA |
| | UUGAGGUAAUGCACAUAUUAAUUAGAAAGAUUUUGUCAUUCCA |
| | CAAUGUAUAUAUACUUAAAAAUAUGUUAUACACAAUAAAUACA |
| | UACAUUAAAAAAUAAGUAAAUGUA |
| 36 | CCCACCCUGCACGCCGGCACCAAACCCUGUCCUCCCACCCCUCCC |
| | CACUCAUCACUAAACAGAGUAAAAUGUGAUGCGAAUUUUCCCGA |
| | CCAACCUGAUUCGCUAGAUUUUUUUUAAGGAAAAGCUUGGAAA |
| | GCCAGGACACAACGCUGCUGCCUGCUUUGUGCAGGGUCCUCCGG |
| | GGCUCAGCCCUGAGUUGGCAUCACCUGCGCAGGGCCCUCUGGGG |
| | CUCAGCCCUGAGCUAGUGUCACCUGCACAGGGCCCUCUGAGGCU |
| | CAGCCCUGAGCUGGCGUCACCUGUGCAGGGCCCUCUGGGGCUCA |
| | GCCCUGAGCUGGCCUCACCUGGGUUCCCCACCCCGGGCUCUCCU |
| | GCCCUGCCCUCCUGCCCGCCCUCCCUCCUGCCUGCGCAGCUCCUU |
| | CCCUAGGCACCUCUGUGCUGCAUCCCACCAGCCUGAGCAAGACG |
| | CCCUCUCGGGGCCUGUGCCGCACUAGCCUCCCUCUCCUCUGUCCC |
| | CAUAGCUGGUUUUUCCCACCAAUCCUCACCUAACAGUUACUUUA |
| | CAAUUAAACUCAAAGCAAGCUCUUCUCCUCAGCUUGGGGCAGCC |
| | AUUGGCCUCUGUCUCGUUUUGGGAAACCAAGGUCAGGAGGCCGU |
| | UGCAGACAUAAAUCUCGGCGACUCGGCCCCGUCUCCUGAGGGUC |
| | CUGCUGGUGACCGGCCUGGACCUUGGCCCUACAGCCCUGGAGGC |
| | CGCUGCUGACCAGCACUGACCCCGACCUCAGAGAGUACUCGCAG |
| | GGGCGCUGGCUGCACUCAAGACCCUCGAGAUUAACGGUGCUAAC |
| | CCCGUCUGCUCCUCCCUCCCGCAGAGACUGGGGCCUGGACUGGA |
| | CAUGAGAGCCCCUUGGUGCCACAGAGGGCUGUGUCUUACUAGAA |
| | ACAACGCAAACCUCUCCUUCCUCAGAAUAGUGAUGUGUUCGACG |
| | UUUUAUCAAAGGCCCCCUUUCUAUGUUCAUGUUAGUUUUGCUCC |
| | UUCUGUGUUUUUUUCUGAACCAUAUCCAUGUUGCUGACUUUUCC |
| | AAAUAAAGGUUUUCACUCCUCUC |
| 37 | AGAGGCCUGCCUCCAGGGCUGGACUGAGGCCUGAGCGCUCCUGC |
| | CGCAGAGCUGGCCGCGCCAAAUAAUGUCUCUGUGAGACUCGAGA |
| | ACUUUCAUUUUUUUCCAGGCUGGUUCGGAUUUGGGGUGGAUUU |
| | UGGUUUUGUUCCCCUCCUCCACUCUCCCCCACCCCCUCCCCGCCC |
| | UUUUUUUUUUUUUUUUUUAAACUGGUAUUUUAUCUUUGAUUCU |
| | CCUUCAGCCCUCACCCCUGGUUCUCAUCUUUCUUGAUCAACAUC |
| | UUUUCUUGCCUCUGUCCCCUUCUCUCAUCUCUUAGCUCCCCUCC |
| | AACCUGGGGGGCAGUGGUGUGGAGAAGCCACAGGCCUGAGAUU |
| | UCAUCUGCUCUCCUUCCUGGAGCCCAGAGGAGGGCAGCAGAAGG |
| | GGGUGGUGUCUCCAACCCCCCAGCACUGAGGAAGAACGGGGCUC |
| | UUCUCAUUUCACCCCUCCCUUUCUCCCCUGCCCCCAGGACUGGGC |
| | CACUUCUGGGUGGGGCAGUGGGUCCCAGAUUGGCUCACACUGAG |
| | AAUGUAAGAACUACAAACAAAAUUUCUAUUAAAUUAAAUUUUG |
| | UGUCUCC |
| 38 | CUCCCUCCAUCCCAACCUGGCUCCCUCCCACCCAACCAACUUUCC |
| | CCCCAACCCGGAAACAGACAAGCAACCCAAACUGAACCCCCUCA |
| | AAAGCCAAAAAAUGGGAGACAAUUUCACAUGGACUUUGGAAAA |
| | UAUUUUUUUCCUUUGCAUUCAUCUCUCAAACUUAGUUUUUAUCU |
| | UUGACCAACCGAACAUGACCAAAAACCAAAAGUGCAUUCAACCU |
| | UACCAAAAAAAAAAAAAAAAAAAGAAUAAAUAAAUAACUUUUU |
| | AAAAAAGGAAGCUUGGUCCACUUGCUUGAAGACCCAUGCGGGGG |
| | UAAGUCCCUUUCUGCCCGUUGGGCUUAUGAAACCCCAAUGCUGC |
| | CCUUUCUGCUCCUUUCUCCACACCCCCCUUGGGGCCUCCCCUCCA |
| | CUCCUUCCCAAAUCUGUCUCCCCAGAAGACACAGGAAACAAUGU |
| | AUUGUCUGCCCAGCAAUCAAAGGCAAUGCUCAAACACCCAAGUG |
| | GCCCCCACCCUCAGCCCGCUCCUGCCCGCCCAGCACCCCCAGGCC |
| | CUGGGGGACCUGGGGUUCUCAGACUGCCAAAGAAGCCUUGCCAU |
| | CUGGCGCUCCCAUGGCUCUUGCAACAUCUCCCCUUCGUUUUUGA |
| | GGGGGUCAUGCCGGGGGAGCCACCAGCCCCUCACUGGGUUCGGA |
| | GGAGAGUCAGGAAGGGCCACGACAAAGCAGAAACAUCGGAUUU |
| | GGGGAACGCGUGUCAAUCCCUUGUGCCGCAGGGCUGGGCGGGAG |
| | AGACUGUUCUGUUCCUUGUGUAACUGUGUUGCUGAAAGACUACC |
| | UCGUUCUUGUCUUGAUGUGUCACCGGGGCAACUGCCUGGGGGCG |
| | GGGAUGGGGGCAGGGUGGAAGCGGCUCCCCAUUUUAUACCAAAG |
| | GUGCUACAUCUAUGUGAUGGGUGGGGUGGGGAGGGAAUCACUG |
| | GUGCUAUAGAAAUUGAGAUGCCCCCCCAGGCCAGCAAAUGUUCC |
| | UUUUUGUUCAAAGUCUAUUUUUAUUCCUUGAUAUUUUUCUUUU |
| | UUUUUUUUUUUUUUUGUGGAUGGGGACUUGUGAAUUUUUCUAA |
| | AGGUGCUAUUUAACAUGGGAGGAGAGCGUGUGCGGCUCCAGCCC |
| | AGCCCGCUGCUCACUUUCCACCCUCUCUCCACCUGCCUCUGGCUU |
| | CUCAGGCCUCUGCUCUCCGACCUCUCUCCUCUGAAACCCUCCUCC |
| | ACAGCUGCAGCCCAUCCUCCCGGCUCCCUCCUAGUCUGUCCUGC |
| | GUCCUCUGUCCCCGGGUUUCAGAGACAACUUCCCAAAGCACAAA |
| | GCAGUUUUUCCCCCUAGGGGUGGGAGGAAGCAAAAGACUCUGUA |
| | CCUAUUUUGUAUGUGUAUAAUAAUUUGAGAUGUUUUUAAUUAU |
| | UUUGAUUGCUGGAAUAAAGCAUGUGGAAAUGACCCAAACAUAA |
| | UCCGCAGUGGCCUCCUAAUUUCCUUCUUUGGAGUUGGGGGAGGG |
| | GUAGACAUGGGGAAGGGGCUUUGGGGUGAUGGGCUUGCCUUCC |
| | AUUCCUGCCCUUUCCCUCCCCACUAUUCUCUUCUAGAUCCCUCC |
| | AUAACCCCACUCCCCUUUCUCUCACCCUUCUUAUACCGCAAACC |
| | UUUCUACUUCCUCUUUCAUUUUCUAUUCUUGCAAUUUCCUUGCA |
| | CCUUUUCCAAAUCCUCUUCUCCCCUGCAAUACCAUACAGGCAAU |
| | CCACGUGCACAACACACACACACACUCUUCACAUCUGGGGUUGU |
| | CCAAACCUCAUACCCACUCCCCUUCAAGCCCAUCCACUCUCCACC |
| | CCCUGGAUGCCCUGCACUUGGUGGCGGUGGGAUGCUCAUGGAUA |
| | CUGGGAGGGUGAGGGGAGUGGAACCCGUGAGGAGGACCUGGGG |
| | GCCUCUCCUUGAACUGACAUGAAGGGUCAUCUGGCCUCUGCUCC |
| | CUUCUCACCCACGCUGACCUCCUGCCGAAGGAGCAACGCAACAG |
| | GAGAGGGGUCUGCUGAGCCUGGCGAGGGUCUGGGAGGGACCAG |
| | GAGGAAGGCGUGCUCCCUGCUCGCUGUCCUGGCCCUGGGGGAGU |
| | GAGGGAGACAGACACCUGGGAGAGCUGUGGGGAAGGCACUCGCA |
| | CCGUGCUCUUGGGAAGGAAGGAGACCUGGCCCUGCUCACCACGG |
| | ACUGGGUGCCUCGACCUCCUGAAUCCCCAGAACACAACCCCCCU |
| | GGGCUGGGGUGGUCUGGGGAACCAUCGUGCCCCCGCCUCCCGCC |
| | UACUCCUUUUUAAGCUU |
| 39 | UUGGCCAGGCCUGACCCUCUUGGACCUUUCUUCUUUGCCGACAA |
| | CCACUGCCCAGCAGCCUCUGGGACCUCGGGGUCCCAGGGAACCC |
| | AGUCCAGCCUCCUGGCUGUUGACUUCCCAUUGCUCUUGGAGCCA |
| | CCAAUCAAAGAGAUUCAAAGAGAUUCCUGCAGGCCAGAGGCGGA |
| | ACACACCUUUAUGGCUGGGGCUCUCCGUGGUGUUCUGGACCCAG |
| | CCCCUGGAGACACCAUUCACUUUUACUGCUUUGUAGUGACUCGU |
| | GCUCUCCAACCUGUCUUCCUGAAAAACCAAGGCCCCCUUCCCCC |
| | ACCUCUUCCAUGGGGUGAGACUUGAGCAGAACAGGGGCUUCCCC |
| | AAGUUGCCCAGAAAGACUGUCUGGGUGAGAAGCCAUGGCCAGAG |
| | CUUCUCCCAGGCACAGGUGUUGCACCAGGGACUUCUGCUUCAAG |
| | UUUUGGGGUAAAGACACCUGGAUCAGACUCCAAGGGCUGCCCUG |
| | AGUCUGGGACUUCUGCCUCCAUGGCUGGUCAUGAGAGCAAACCG |
| | UAGUCCCCUGGAGACAGCGACUCCAGAGAACCUCUUGGGAGACA |
| | GAAGAGGCAUCUGUGCACAGCUCGAUCUUCUACUUGCCUGUGGG |
| | GAGGGGAGUGACAGGUCCACACACCACACUGGGUCACCCUGUCC |
| | UGGAUGCCUCUGAAGAGAGGGACAGACCGUCAGAAACUGGAGA |
| | GUUUCUAUUAAAGGUCAUUUAAACCA |
| 40 | UCCUCCGGGACCCCAGCCCUCAGGAUUCCUGAUGCUCCAAGGCG |
| | ACUGAUGGGCGCUGGAUGAAGUGGCACAGUCAGCUUCCCUGGGG |
| | GCUGGUGUCAUGUUGGGCUCCUGGGGCGGGGGCACGGCCUGGCA |
| | UUUCACGCAUUGCUGCCACCCCAGGUCCACCUGUCUCCACUUUC |
| | ACAGCCUCCAAGUCUGUGGCUCUUCCCUUCUGUCCUCCGAGGGG |
| | CUUGCCUUCUCUCGUGUCCAGUGAGGUGCUCAGUGAUCGGCUUA |
| | ACUUAGAGAAGCCCGCCCCCUCCCCUUCUCCGUCUGUCCCAAGA |
| | GGGUCUGCUCUGAGCCUGCGUUCCUAGGUGGCUCGGCCUCAGCU |
| | GCCUGGGUUGUGGCCGCCCUAGCAUCCUGUAUGCCCACAGCUAC |
| | UGGAAUCCCCGCUGCUGCUCCGGGCCAAGCUUCUGGUUGAUUAA |
| | UGAGGGCAUGGGGUGGUCCCUCAAGACCUUCCCCUACCUUUUGU |
| | GGAACCAGUGAUGCCUCAAAGACAGUGUCCCCUCCACAGCUGGG |
| | UGCCAGGGGCAGGGGAUCCUCAGUAUAGCCGGUGAACCCUGAUA |
| | CCAGGAGCCUGGGCCUCCCUGAACCCCUGGCUUCCAGCCAUCUC |
| | AUCGCCAGCCUCCUCCUGGACCUCUUGGCCCCCAGCCCCUUCCCC |
| | ACACAGCCCCAGAAGGGUCCCAGAGCUGACCCCACUCCAGGACC |
| | UAGGCCCAGCCCCUCAGCCUCAUCUGGAGCCCCUGAAGACCAGU |
| | CCCACCCACCUUUCUGGCCUCAUCUGACACUGCUCCGCAUCCUGC |
| | UGUGUGUCCUGUUCCAUGUUCCGGUUCCAUCCAAAUACACUUUC |
| | UGGAACAAA |
| 41 | GCUGGAGCCUCGGUGGCCAUGCUUCUUGCCCCUUGGGCCUCCCC |
| | CCAGCCCCUCCUCCCCUUCCUGCACCCGUACCCCCGUGGUCUUUG |
| | AAUAAAGUCUGAGUGGGCGGC |

In one embodiment, the mRNA comprises a 5′ UTR comprising a sequence selected from SEQ. ID. NOS.: 6-23, a coding sequence encoding an rOAS1b amino acid sequence selected from SEQ. ID. NOS.: 1-3, and a 3′ UTR comprising a sequence selected from SEQ. ID. NOS.: 24-41.

In one embodiment, the mRNA comprises a 5′ UTR comprising the sequence of SEQ. ID. NO. 6, a coding sequence encoding an rOAS1b amino acid sequence selected from SEQ. ID. NOS.: 1-3, a 3′ UTR comprising the sequence of SEQ. ID. NO. 24.

In one embodiment, the mRNA comprises a 5′ UTR comprising a sequence selected from SEQ. ID. NOS.: 6-23, a coding sequence of SEQ. ID. NO.: 4, and a 3′ UTR comprising a sequence selected from SEQ. ID. NOS.: 24-41.

In one embodiment, the mRNA comprises a 5′ UTR comprising the sequence of SEQ. ID. NO. 6, a coding sequence of SEQ. ID. NO.: 4, and a 3′ UTR comprising the sequence of SEQ. ID. NO.: 24.

In one embodiment, the mRNA comprises a sequence of SEQ. ID. NO.: 42, or a variant thereof that is 80%, 85%, 90%, 95%, 96%, 97%, 98%, 99% or more homologous:

(SEQ. ID. NO.: 42)
GGAAAUAAGAGAGAAAAGAAGAGUAAGAAGAAAUAUAAGAGCCACCAUGGA
GCAAGAUCUUAGAUCCAUACCAGCUUCCAAGCUCGAUAAGUUCAUUGAGAA
UCAUCUGCCCGACACCUCCUUUUGCGCUGACCUUAGAGAGGUCAUAGAUGC
CUUGUGCGCCCUUCUCAAAGACCGAUCAUUUAGAGGUCCUGUCCGGCGCAU
GAGAGCAUCAAAGGGGGUCAAGGGUAAAGGGACCACACUUAAGGGGAGGUC
UGACGCCGACCUGGUCGUCUUCCUCAACAACUUGACCAGCUUUGAGGAUCA
GCUCAAUCAACAAGGUGUACUUAUAAAAGAGAUCAAAAAACAACUCUGUGA
AGUGCAGCACGAACGCAGGUGUGGUGUUAAGUUUGAAGUUCAUUCACUUCG
CAGUCCCAAUUCUCGGGCUCUCAGCUUUAAGCUGUCAGCCCCCGACUUGCU
CAAAGAAGUGAAGUUUGAUGUGCUGCCCGCAUACGACCUUCUCGACCAUCU
CAACAUACUGAAGAAACCAAACCAACAGUUUUACGCAAACCUCAUUAGCGG
ACGAACUCCACCUGGCAAAGAGGGGAAAUUGAGCAUAUGUUUCAUGGGACU
UCGGAAGUACUUCCUGAACUGCCGCCCCACUAAACUGAAGAGGCUUAUUAG
GCUCGUCACUCACUGGUAUCAAUUGUGUAAGGAGAAGCUGGGGGAUCCCCU
CCCACCUCAAUACGCACUUGAACUCUUGACCGUUUACGCAUGGGAGUACGG
AAGUCGGGUCACUAAAUUCAAUACAGCUCAGGGCUUUCGGACCGUGUUGGA
ACUCGUAACUAAGUAUAAGCAGUUGAGGAUAUACUGGACUGUAUAUUAUGA
UUUUAGACACCAAGAGGUUUCAGAGUACCUUCAUCAACAGCUUAAAAAAGA
UCGACCCGUUAUUCUGGACCCUGCCGACCCCACACGAAAUAUCGCCGGCCU
UAACCCUAAGGACUGGAGACGACUUGCUGGGGAAGCAGCCACUUGGCUUCA
GUAUCCCUGCUUCAAAUAUAGAGACGGCAGCCCUGUGUGUUCUUGGGAAGU
CCCCACAGAGGUCGGGGUCCCCAUGAAAUAUCUCUUCUGUCGCAUCUUCUG
GCUCUUGUUUUGGAGUCUGUUCCACUUCAUCUUUGGUAAAACCUCCUCAGG
UUAGUGAUAAGCUGCCUUCUGCGGGGCUUGCCUUCUGGCCAUGCCCUUCUU
CUCUCCCUUGCACCUGUACCUCUUGGUCUUUGAAUAAAGCCUGAGUAGGAA
GGCGGCCGCAAAAA.

### 5′ Terminal Cap

The 5′ terminal cap structure of natural mRNA is involved in nuclear transport, increasing mRNA stability and binds the mRNA Cap Binding Protein (CBP), which is responsible for mRNA stability in the cell and translation competency through the association of CBP with poly(A) binding protein to form the mature cyclic mRNA species. The 5′ terminal cap is operably linked to the 5′ end of the mRNA as described herein.

Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

In some embodiments, mRNA of the present invention may be designed to incorporate a cap moiety. Modifications to the polynucleotides of the present invention may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, Mass.) may be used with a-thio-guanosine nucleotides according to the manufacturer's instructions to create a phosphorothioate linkage in the 5′-ppp-5′ cap. Additional modified guanosine nucleotides may be used such as a-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide which functions as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which may equivalently be designated 3′-O-Me-m7G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

In one embodiment, the cap is a dinucleotide cap analog. As a non-limiting example, the dinucleotide cap analog may be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m3-0 G(5′)ppp(5′)G cap analog (See e.g., the various cap analogs and the methods of synthesizing cap analogs described in Kore et al. Bioorganic & Medicinal Chemistry 2013 21:4570-4574, herein incorporated by reference in its entirety). In another embodiment, a cap analog of the present invention is a 4-chloro/bromophenoxyethyl analog.

While cap analogs allow for the concomitant capping of a polynucleotide or a region thereof, in an in vitro transcription reaction up to 20% of transcripts can remain uncapped. This, as well as the structural differences of a cap analog from an endogenous 5′-cap structures of nucleic acids produced by the endogenous, cellular transcription machinery, may lead to reduced translational competency and reduced cellular stability. mRNA of the invention may also be capped post-manufacture (whether by IVT or chemical synthesis), using enzymes, in order to generate more authentic 5′-cap structures, for example to closely mirror or mimic, either structurally or functionally, an endogenous or wild type feature.

Non-limiting examples of such 5′cap structures of the present invention are those which, among other things, have enhanced binding of cap binding proteins, increased half-life, reduced susceptibility to 5′ endonucleases and/or reduced 5′ decapping, as compared to synthetic 5′ cap structures known in the art (or to a wild-type, natural or physiological 5′ cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′ cap analog structures known in the art. Cap structures include, but are not limited to, 7mG(5′)ppp(5′)N,pN2p (cap 0), 7mG(5′)ppp(5′)NlmpNp (cap 1), and 7mG(5′)-ppp(5′)NlmpN2mp (cap 2).

5′-terminal caps may include endogenous caps or cap analogs. A 5′ terminal cap may comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### 3′ Polyadenylation

During RNA processing, a long chain of adenine nucleotides, known as the poly(A) tail, may be added to the mRNA molecule in order to increase stability. Immediately after transcription, the 3′ end of the transcript may be cleaved to free a 3′ hydroxyl. Then poly(A) polymerase adds a chain of adenine nucleotides to the RNA. The process, called polyadenylation, adds a poly(A) tail that can be between, for example, approximately 80 to approximately 250 residues long, including approximately 80, 90, 100, 110, 120, 130, 140, 150, 160, 170, 180, 190, 200, 210, 220, 230, 240 or 250 residues long. The poly(A) tail is operably linked to 3′ end of the mRNA as described herein. Poly(A) tails may also be added after the construct is exported from the nucleus.

Terminal groups on the poly(A) tail may be incorporated for stabilization into mRNA of the present invention. Polynucleotides of the present invention may include des-3′ hydroxyl tails. They may also include structural moieties or 2′-O-methyl modifications as taught by Junjie Li, et al. (Current Biology, Vol. 15, 1501-1507, Aug. 23, 2005, herein incorporated by reference in its entirety).

The mRNA of the present invention may be designed to encode transcripts with alternative poly(A) tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi:10.1038/nrm3645, herein incorporated by reference in its entirety).

Unique poly(A) tail lengths provide certain advantages to the mRNA of the present invention. Generally, the length of a poly(A) tail, when present, is greater than 30 nucleotides in length. In another embodiment, the poly(A) tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the polynucleotide or region thereof includes from about 30 to about 3,000 nucleotides.

In one embodiment, the poly(A) tail is designed relative to the length of the overall polynucleotide or the length of a particular region of the polynucleotide. This design may be based on the length of the coding region, the length of a particular feature or region or based on the length of the ultimate product expressed.

In this context the poly(A) tail may be 10, 20, 30, 40, 50, 60, 70, 80, 90, or 100% greater in length than the polynucleotide or feature thereof. The poly(A) tail may also be designed as a fraction of the polynucleotides to which it belongs. In this context, the poly(A) tail may be 10, 20, 30, 40, 50, 60, 70, 80, or 90% or more of the total length of the construct, a construct region or the total length of the construct minus the poly(A) tail. Further, engineered binding sites and conjugation of polynucleotides for Poly(A) binding protein may enhance expression.

Additionally, multiple distinct polynucleotides may be linked together via the PABP (Poly-A binding protein) through the 3′-end using modified nucleotides at the 3′-terminus of the poly(A) tail.

In one embodiment, the mRNA of the present invention is designed to include a poly(A) G Quartet region. The G-quartet is a cyclic hydrogen bonded array of four guanine nucleotides that can be formed by G-rich sequences in both DNA and RNA. In this embodiment, the G-quartet is incorporated at the end of the poly(A) tail. The resultant polynucleotide is assayed for stability, protein production and other parameters including half-life at various time points. It has been discovered that the poly(A) G quartet results in protein production from an mRNA equivalent to at least 75% of that seen using a poly(A) tail of 120 nucleotides alone.

### Start Codon Region

In some embodiments, the mRNA of the present invention may have regions that are analogous to or function like a start codon region.

In one embodiment, the translation of the mRNA may initiate on a codon which is not the start codon AUG. Translation of the polynucleotide may initiate on an alternative start codon such as, but not limited to, ACG, AGG, AAG, CUG, GUG, AUA, AUU, UUG (see Touriol et al. Biology of the Cell 95 (2003) 169-178 and Matsuda and Mauro PLoS ONE, 2010 5:11, herein incorporated by reference in its entirety). As a non-limiting example, the translation of a polynucleotide begins on the alternative start codon ACG. As another non-limiting example, polynucleotide translation begins on the alternative start codon CUG. As yet another non-limiting example, the translation of a polynucleotide begins on the alternative start codon GUG.

### Stop Codon Region

In one embodiment, the mRNA of the present invention may include at least two stop codons before the 3′untranslated region (UTR). The stop codon may be selected from UGA, UAA and UAG. In one embodiment, the polynucleotides of the present invention include the stop codon UGA and one additional stop codon. In a further embodiment the addition stop codon may be UAA. In another embodiment, the polynucleotides of the present invention include three stop codons.

### Codon Optimization

The coding region of the mRNA of the present invention and their regions or parts or subregions may be codon optimized. Codon optimization methods are known in the art and may be useful in efforts to achieve one or more of several goals. These goals include to match codon frequencies in target and host organisms to ensure proper folding, to bias GC content to increase mRNA stability or reduce secondary structures, to minimize tandem repeat codons or base runs that may impair gene construction or expression, to customize translational control regions, to insert or remove protein trafficking sequences, to remove/add post translation modification sites in encoded protein (e.g. glycosylation sites), to add or remove or shuffle protein domains, to insert or delete restriction sites, to modify ribosome binding sites and mRNA degradation sites, to adjust translational rates to allow the various domains of the protein to fold properly, or to reduce or eliminate problematic secondary structures within the polynucleotide. Codon optimization tools, algorithms and services are known in the art, non-limiting examples of which include services from GeneArt (Life Technologies), DNA2.0 (Menlo Park Calif.) and/or proprietary methods. In one embodiment, the coding region sequence is optimized using optimization algorithms. mRNA Codon options for each amino acid are given in Table 3.

TABLE 3
Codon Options for Each Amino Acid

| Amino Acid | Single Letter Code | Codon Options |
|---|---|---|
| Isoleucine | I | AUU, AUC, AUA |
| Leucine | L | CUU, CUC, CUA, CUG, UUA, UUG |
| Valine | V | GUU, GUC, GUA, GUG |
| Phenylalanine | F | UUU, UUC |
| Methionine | M | AUG |
| Cysteine | C | UGU, UGC |
| Alanine | A | GCU, GCC, GCA, GCG |
| Glycine | G | GGU, GGC, GGA, GGG |
| Proline | P | CCU, CCC, CCA, CCG |
| Threonine | T | ACU, ACC, ACA, ACG |
| Serine | S | UCU, UCC, UCA, UCG, AGU, AGC |
| Tyrosine | Y | UAU, UAC |
| Tryptophan | W | UGG |
| Glutamine | Q | CAA, CAG |
| Asparagine | N | AAU, AAC |
| Histidine | H | CAU, CAC |
| Glutamic Acid | E | GAA, GAG |
| Aspartic Acid | D | GAU, GAC |
| Lysine | K | AAA, AAG |
| Arginine | R | CGU, CGC, CGA, CGG, AGA, AGG |
| Selenocysteine | Sec | UGA in mRNA in presence of SECIS |
| Stop Codons | Stop | UAA, UAG, UGA |

### Pharmaceutical Compositions

The mRNA of the present invention can be formulated using one or more excipients to increase stability, increase cell transfection, permit sustained or delayed release, alter biodistribution, increase in vivo translation of the encoded protein, and/or alter the in vivo release profile of the encoded protein. In addition to traditional excipients such as any and all solvents, dispersion media, diluents or other liquid vehicles, dispersion or suspension aids, surface active agents, isotonic agents, thickening or emulsifying agents, and preservatives, excipients of the present invention can include, without limitation, lipidoids, liposomes, lipid nanoparticles, polymers, lipoplexes, core-shell nanoparticles, and combinations thereof.

Accordingly, the formulations of the present invention can include one or more excipients, each in an amount that may increase the stability of polynucleotide, increase cell transfection by the polynucleotide, increase the expression of the encoded protein, or alter the release profile of the encoded protein.

Formulations as described herein may be prepared by any method known in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the mRNA. The amount of the mRNA is generally equal to the dosage of the mRNA to be delivered and/or a convenient fraction of such a dosage such as, for example, one-half or one-third of such a dosage.

Relative amounts of the mRNA, the excipient, and/or any other additional ingredients in the pharmaceutical composition with the present disclosure may vary, depending upon the identity, size, and/or condition of the subject being treated and further depending upon the route by which the composition is being administered. For example, the composition may comprise between 0.1% and 99% (w/w) of the mRNA. By way of example, the composition may contain between 0.1% and 100%, e.g., between 0.5% and 50%, between 1% and 30%, between 5% and 80%, and at least 80% (w/w) of the mRNA.

Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, Md. 2006, herein incorporated by reference in its entirety). The use of a conventional excipient medium may be contemplated within the scope of the present disclosure, except as insofar as a convention excipient medium may be incompatible with a substance or its derivatives, such as by producing an undesirable biological effect or otherwise interacting in a deleterious manner with any of the components of the pharmaceutical composition.

Pharmaceutically acceptable excipients include, but are not limited to, inert diluents, surface active agents and/or emulsifiers, preservatives, buffering agents, lubricating agents, and/or oils. Such excipients may optionally be including in the pharmaceutical formulations of the invention.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions. The composition may also include excipients such as cocoa butter and suppository waxes, coloring agents, coating agents, sweetening, flavoring, and/or perfuming agents.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); amino acids (e.g., glycine); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Oxidation is a potential degradation pathway for mRNA, especially for liquid mRNA formulations. In order to prevent oxidation, antioxidants can be added to the formulation. Exemplary antioxidants include, but are not limited to, alpha tocopherol, ascorbic acid, acorbyl palmitate, benzyl alcohol, butylated hydroxyanisole, EDTA, m-cresol, methionine, butylated hydroxytoluene, monothioglycerol, potassium metabisulfite, propionic acid, propyl gallate, sodium ascorbate, sodium bisulfite, sodium metabisulfite, thioglycerol and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybcnzoatc, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

In some embodiments, the pH of the mRNA solutions is maintained between pH 5 and pH 8 to improve stability. Exemplary buffers to control pH may include, but are not limited to sodium phosphate, sodium citrate, sodium succinate, histidine (or histidine-HCl), sodium carbonate, and/or sodium malate. In another embodiment, the exemplary buffers listed above may be used with additional monovalent counterions (including, but not limited to potassium). Divalent cations may also be used as buffer counterions; however, these are not preferred due to complex formation and/or mRNA degradation.

Exemplary buffering agents may also include, but are not limited to, citrate buffer solutions, acetate buffer solutions, phosphate buffer solutions, ammonium chloride, calcium carbonate, calcium chloride, calcium citrate, calcium glucionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

Exemplary additives include physiologically biocompatible buffers (e.g., trimethylamine hydrochloride), addition of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). In addition, antioxidants and suspending agents can be used.

In certain embodiments, the pharmaceutical composition for administration further includes the polynucleotide described herein and optionally comprises one or more of a phosphoglyceride; phosphatidylcholine; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohol such as polyethylene glycol (PEG); polyoxyethylene-9-lauryl ether; a surface active fatty acid, such as palmitic acid or oleic acid; fatty acid; fatty acid monoglyceride; fatty acid diglyceride; fatty acid amide; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebroside; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipid; synthetic and/or natural detergent having high surfactant properties; deoxycholate; cyclodextrin; chaotropic salt; ion pairing agent; glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid; pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan, mannitol, sorbitol, xylitol, erythritol, maltitol, and lactitol, a pluronic polymer, polyethylene, polycarbonate (e.g. poly(1,3-dioxan-2one)), polyanhydride (e.g. poly(sebacic anhydride)), polypropylfumerate, polyamide (e.g. polycaprolactam), polyacetal, polyether, polyester (e.g., polylactide, polyglycolide, polylactide-co-glycolide, polycaprolactone, polyhydroxyacid (e.g. poly((β-hydroxyalkanoate))), poly(orthoester), polycyanoacrylate, polyvinyl alcohol, polyurethane, polyphosphazene, polyacrylate, polymethacrylate, polyurea, polystyrene, and polyamine, polylysine, polylysine-PEG copolymer, and poly(ethyleneimine), poly(ethylene imine)-PEG copolymer, glycerol monocaprylocaprate, propylene glycol, Vitamin E TPGS (also known as d-a-Tocopheryl polyethylene glycol 1000 succinate), gelatin, titanium dioxide, polyvinylpyrrolidone (PVP), hydroxypropyl methyl cellulose (HPMC), hydroxypropyl cellulose (HPC), methyl cellulose (MC), block copolymers of ethylene oxide and propylene oxide (PEO/PPO), polyethyleneglycol (PEG), sodium carboxymethylcellulose (NaCMC), hydroxypropylmethyl cellulose acetate succinate (HPMCAS).

Pharmaceutical compositions, and methods of manufacturing such compositions, suitable for administration as contemplated herein are known in the art. Examples of known techniques include, for example, U.S. Pat. Nos. 4,983,593, 5,013,557, 5,456,923, 5,576,025, 5,723,269, 5,858,411, 6,254,889, 6,303,148, 6,395,302, 6,497,903, 7,060,296, 7,078,057, 7,404,828, 8,202,912, 8,257,741, 8,263,128, 8,337,899, 8,431,159, 9,028,870, 9,060,938, 9,211,261, 9,265,731, 9,358,478, and 9,387,252, incorporated by reference herein.

Additional non-limiting examples of drug delivery devices and methods include, for example, US20090203709 titled “Pharmaceutical Dosage Form For Oral Administration Of Tyrosine Kinase Inhibitor” (Abbott Laboratories); US20050009910 titled “Delivery of an active drug to the posterior part of the eye via subconjunctival or periocular delivery of a prodrug”, US 20130071349 titled “Biodegradable polymers for lowering intraocular pressure”, U.S. Pat. No. 8,481,069 titled “Tyrosine kinase microspheres”, U.S. Pat. No. 8,465,778 titled “Method of making tyrosine kinase microspheres”, U.S. Pat. No. 8,409,607 titled “Sustained release intraocular implants containing tyrosine kinase inhibitors and related methods”, U.S. Pat. No. 8,512,738 and US 2014/0031408 titled “Biodegradable intravitreal tyrosine kinase implants”, US 2014/0294986 titled “Microsphere Drug Delivery System for Sustained Intraocular Release”, U.S. Pat. No. 8,911,768 titled “Methods For Treating Retinopathy With Extended Therapeutic Effect” (Allergan, Inc.); U.S. Pat. No. 6,495,164 titled “Preparation of injectable suspensions having improved injectability” (Alkermes Controlled Therapeutics, Inc.); WO 2014/047439 titled “Biodegradable Microcapsules Containing Filling Material” (Akina, Inc.); WO 2010/132664 titled “Compositions And Methods For Drug Delivery” (Baxter International Inc. Baxter Healthcare SA); US20120052041 titled “Polymeric nanoparticles with enhanced drug loading and methods of use thereof” (The Brigham and Women's Hospital, Inc.); US20140178475, US20140248358, and US20140249158 titled “Therapeutic Nanoparticles Comprising a Therapeutic Agent and Methods of Making and Using Same” (BIND Therapeutics, Inc.); U.S. Pat. No. 5,869,103 titled “Polymer microparticles for drug delivery” (Danbiosyst UK Ltd.); U.S. Pat. No. 8,628,801 titled “Pegylated Nanoparticles” (Universidad de Navarra); US2014/0107025 titled “Ocular drug delivery system” (Jade Therapeutics, LLC); U.S. Pat. No. 6,287,588 titled “Agent delivering system comprised of microparticle and biodegradable gel with an improved releasing profile and methods of use thereof”, U.S. Pat. No. 6,589,549 titled “Bioactive agent delivering system comprised of microparticles within a biodegradable to improve release profiles” (Macromed, Inc.); U.S. Pat. Nos. 6,007,845 and 5,578,325 titled “Nanoparticles and microparticles of non-linear hydrophilichydrophobic multiblock copolymers” (Massachusetts Institute of Technology); US20040234611, US20080305172, US20120269894, and US20130122064 titled “Ophthalmic depot formulations for periocular or subconjunctival administration (Novartis Ag); U.S. Pat. No. 6,413,539 titled “Block polymer” (Poly-Med, Inc.); US 20070071756 titled “Delivery of an agent to ameliorate inflammation” (Peyman); US 20080166411 titled “Injectable Depot Formulations And Methods For Providing Sustained Release Of Poorly Soluble Drugs Comprising Nanoparticles” (Pfizer, Inc.); U.S. Pat. No. 6,706,289 titled “Methods and compositions for enhanced delivery of bioactive molecules” (PR Pharmaceuticals, Inc.); and U.S. Pat. No. 8,663,674 titled “Microparticle containing matrices for drug delivery” (Surmodics), herein incorporated by reference in their entirety.

### Lipidoid Formulations

The synthesis of lipidoids has been extensively described and formulations containing these compounds are particularly suited for delivery of polynucleotides (see Mahon et al., Bioconjug Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001, and US 2016/0317647, herein incorporated by reference in their entirety).

While these lipidoids have been used to effectively deliver double stranded small interfering RNA molecules in rodents and non-human primates (see Akinc et al., Nat Biotechnol. 2008 26:561-569; Frank-Kamenetsky et al., Proc Natl Acad Sci USA. 2008 105:11915-11920; Akinc et al., Mol Ther. 2009 17:872-879; Love et al., Proc Natl Acad Sci USA. 2010 107:1864-1869; Leuschner et al., Nat Biotechnol. 2011 29:1005-1010, herein incorporated by reference in their entirety), the present disclosure describes their formulation and use in delivering mRNA contained therein.

Complexes, micelles, liposomes or particles can be prepared containing these lipidoids and therefore, can result in an effective delivery of the polynucleotide, as judged by the production of an encoded protein, following the injection of a lipidoid formulation via localized and/or systemic routes of administration. Lipidoid complexes of polynucleotides can be administered by various means including, but not limited to, intravenous, intramuscular, or subcutaneous routes.

In vivo delivery of nucleic acids may be affected by many parameters, including, but not limited to, the formulation composition, nature of particle PEGylation, degree of loading, polynucleotide to lipid ratio, and biophysical parameters such as, but not limited to, particle size (Akinc et al., Mol Ther. 2009 17:872-879, herein incorporated by reference in its entirety). As an example, small changes in the anchor chain length of poly(ethylene glycol) (PEG) lipids may result in significant effects on in vivo efficacy. Formulations with the different lipidoids, including, but not limited to penta[3-(1-laurylaminopropionyl)]-triethylenetetramine hydrochloride (TETA-SLAP; aka 98N12-5, see Murugaiah et al., Analytical Biochemistry, 401:61 (2010), herein incorporated by reference in its entirety, and MD1, can be tested for in vivo activity.

The lipidoid referred to herein as “98N12-5” is disclosed by Akinc et al., Mol Ther. 2009 17:872-879, herein incorporated by reference in its entirety. The lipidoid referred to herein as “C12-200” is disclosed by Love et al., Proc Natl Acad Sci USA. 2010 107:1864-1869 and Liu and Huang, Molecular Therapy. 2010 669-670, herein incorporated by reference in their entirety. The lipidoid formulations can include particles comprising either 3 or 4 or more components in addition to polynucleotides. As an example, formulations with certain lipidoids, include, but are not limited to, 98N12-5 and may contain 42% lipidoid, 48% cholesterol and 10% PEG (C14 alkyl chain length). As another example, formulations with certain lipidoids, include, but are not limited to, C12-200 and may contain 50% lipidoid, 10% disteroylphosphatidyl choline, 38.5% cholesterol, and 1.5% PEG-DMG.

In one embodiment, a polynucleotide formulated with a lipidoid for systemic intravenous administration can target the liver. For example, a final optimized intravenous formulation using polynucleotides, and comprising a lipid molar composition of 42% 98N12-5, 48% cholesterol, and 10% PEG-lipid with a final weight ratio of about 7.5 to 1 total lipid to polynucleotides, and a C14 alkyl chain length on the PEG lipid, with a mean particle size of roughly 50-60 nm, can result in the distribution of the formulation to be greater than 90% to the liver. (see, Akinc et al., Mol Ther. 2009 17:872-879, herein incorporated by reference in its entirety). In another example, an intravenous formulation using a C12-200 (see U.S. published international application WO2010129709, which is herein incorporated by reference in its entirety) lipidoid may have a molar ratio of 50/10/38.5/1.5 of C12-200/disteroylphosphatidyl choline/cholesterol/PEG-DMG, with a weight ratio of 7 to 1 total lipid to polynucleotides, and a mean particle size of 80 nm may be effective to deliver polynucleotides to hepatocytes (see, Love et al., Proc Natl Acad Sci USA. 2010 107:1864, herein incorporated by reference in its entirety). In another embodiment, an MD1 lipidoid-containing formulation may be used to effectively deliver polynucleotides to hepatocytes in vivo.

The characteristics of optimized lipidoid formulations for intramuscular or subcutaneous routes may vary significantly depending on the target cell type and the ability of formulations to diffuse through the extracellular matrix into the blood stream. While a particle size of less than 150 nm may be desired for effective hepatocyte delivery due to the size of the endothelial fenestrae (see, Akinc et al., Mol Ther. 2009 17:872-879, herein incorporated by reference in its entirety), use of a lipidoid-formulated compositions to deliver the formulation to other cells types including, but not limited to, endothelial cells, myeloid cells, and muscle cells may not be similarly size-limited.

Use of lipidoid formulations to deliver RNA in vivo to other non-hepatocyte cells such as myeloid cells and endothelium has been reported (see Akinc et al., Nat Biotechnol. 2008 26:561-569; Leuschner et al., Nat Biotechnol. 2011 29:1005-1010; Cho et al. Adv. Funct. Mater. 2009 19:3112-3118; 8th International Judah Folkman Conference, Cambridge, Mass. Oct. 8-9, 2010, herein incorporated by reference in their entirety). For effective delivery to myeloid cells, such as monocytes, lipidoid formulations may have a similar component molar ratio. Different ratios of lipidoids and other components including, but not limited to, disteroylphosphatidyl choline, cholesterol and PEG-DMG, may be used to optimize the formulation of the mRNA composition for delivery to different cell types including, but not limited to, hepatocytes, myeloid cells, muscle cells, etc. For example, the component molar ratio may include, but is not limited to, 50% C12-200, 10% disteroylphosphatidyl choline, 38.5% cholesterol, and 1.5% PEG-DMG (see Leuschner et al., Nat Biotechnol 2011 29:1005-1010, herein incorporated by reference in its entirety). The use of lipidoid formulations for the localized delivery of nucleic acids to cells (such as, but not limited to, adipose cells and muscle cells) via either subcutaneous or intramuscular delivery, may not require all of the formulation components desired for systemic delivery, and as such may comprise only the lipidoid and the mRNA.

Combinations of different lipidoids may be used to improve the efficacy of polynucleotides directed protein production as the lipidoids may be able to increase cell transfection by the mRNA; and/or increase the translation of encoded protein (see Whitehead et al., Mol. Ther. 2011, 19:1688-1694, herein incorporated by reference in its entirety).

### Liposomal, Lipoplex, and Lipid Nanoparticle Formulations

The mRNA of the invention can be formulated using one or more liposomes, lipoplexes, or lipid nanoparticles. In one embodiment, pharmaceutical compositions include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unilamellar vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, the contents of each of which are herein incorporated by reference in its entirety.

In one embodiment, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.).

In one embodiment, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104, herein incorporated by reference in their entirety). The original manufacture method by Wheeler et al. was a detergent dialysis method, which was later improved by Jeffs et al. and is referred to as the spontaneous vesicle formation method. The liposome formulations are composed of 3 to 4 lipid components in addition to the polynucleotide. As an example a liposome can contain, but is not limited to, 55% cholesterol, 20% distcroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In some embodiments, liposome formulations may comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In a preferred embodiment, formulations may comprise a percentage of cholesterol selected from the group consisting of 28.5%, 31.5%, 33.5%, 36.5%, 37.0%, 38.5%, 39.0% and 43.5%. In some embodiments, formulations may comprise from about 5.0% to about 10.0% DSPC and/or from about 7.0% to about 15.0% DSPC.

In one embodiment, pharmaceutical compositions may include liposomes which may be formed to deliver mRNA of the present invention. The polynucleotide may be encapsulated by the liposome and/or it may be contained in an aqueous core which may then be encapsulated by the liposome (see International Pub. Nos. WO2012031046, WO2012031043, WO2012030901 and WO2012006378 and US Patent Publication No. US20130189351, US20130195969 and US20130202684; the contents of each of which are herein incorporated by reference in their entirety).

In another embodiment, liposomes may be formulated for targeted delivery. As a non-limiting example, the liposome may be formulated for targeted delivery to the liver. The liposome used for targeted delivery may include, but is not limited to, the liposomes described in and methods of making liposomes described in US Patent Publication No. US20130195967, the contents of which are herein incorporated by reference in its entirety.

In another embodiment, the mRNA of the present invention may be formulated in a cationic oil-in-water emulsion where the emulsion particle comprises an oil core and a cationic lipid which can interact with the polynucleotide anchoring the molecule to the emulsion particle (see International Pub. No. WO2012006380; herein incorporated by reference in its entirety).

In one embodiment, the mRNA of the present invention may be formulated in a water-in-oil emulsion comprising a continuous hydrophobic phase in which the hydrophilic phase is dispersed. As a non-limiting example, the emulsion may be made by the methods described in International Publication No. WO201087791, the contents of which are herein incorporated by reference in its entirety.

In another embodiment, the lipid formulation may include at least a cationic lipid, a lipid which may enhance transfection and a least one lipid which contains a hydrophilic head group linked to a lipid moiety (International Pub. No. WO2011076807 and U.S. Pub. No. 20110200582; the contents of each of which is herein incorporated by reference in their entirety). In another embodiment, the mRNA of the present invention may be formulated in a lipid vesicle which may have crosslinks between functionalized lipid bilayers (see U.S. Pub. No. 20120177724, the contents of which is herein incorporated by reference in its entirety).

In one embodiment, the polynucleotides may be formulated in a liposome as described in International Patent Publication No. WO2013086526, the contents of which is herein incorporated by reference in their entirety. The mRNA may be encapsulated in a liposome using reverse pH gradients and/or optimized internal buffer compositions as described in International Patent Publication No. WO2013086526, the contents of which is herein incorporated by reference in its entirety.

In one embodiment, the mRNA pharmaceutical compositions may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713, herein incorporated by reference in its entirety) and hyaluronan-coated liposomes (Quiet Therapeutics, Israel).

In one embodiment, the cationic lipid may be a low molecular weight cationic lipid such as those described in US Patent Application No. 20130090372, the contents of which are herein incorporated by reference in its entirety.

In one embodiment, the mRNA may be formulated in a lipid vesicle which may have crosslinks between functionalized lipid bilayers.

In one embodiment, the mRNA may be formulated in a liposome comprising a cationic lipid. The liposome may have a molar ratio of nitrogen atoms in the cationic lipid to the phosphates in the RNA (N:P ratio) of between 1:1 and 20:1 as described in International Publication No. WO2013006825, herein incorporated by reference in its entirety. In another embodiment, the liposome may have a N:P ratio of greater than 20:1 or less than 1:1.

In one embodiment, the mRNA may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Pub. No. WO2012013326 or US Patent Pub. No. US20130142818; each of which is herein incorporated by reference in its entirety. In another embodiment, the mRNA may be formulated in a lipid-polycation complex which may further include a non-cationic lipid such as, but not limited to, cholesterol or dioleoyl phosphatidylethanolamine (DOPE).

In one embodiment, the mRNA may be formulated in an aminoalcohol lipidoid. Aminoalcohol lipidoids which may be used in the present invention may be prepared by the methods described in U.S. Pat. No. 8,450,298, herein incorporated by reference in its entirety.

The liposome formulation may be influenced by, but not limited to, the selection of the cationic lipid component, the degree of cationic lipid saturation, the nature of the PEGylation, ratio of all components and biophysical parameters such as size. In one example by Semple et al. (Nature Biotech. 2010 28:172-176, herein incorporated by reference in its entirety), the liposome formulation was composed of 57.1% cationic lipid, 7.1% dipalmitoylphosphatidylcholine, 34.3% cholesterol, and 1.4% PEG-c-DMA. As another example, changing the composition of the cationic lipid could more effectively deliver siRNA to various antigen presenting cells (Basha et al. Mol Ther. 2011 19:2186-2200, herein incorporated by reference in its entirety). In some embodiments, liposome formulations may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to mRNA in liposomes may be from about 5:1 to about 20:1, from about 10:1 to about 25:1, from about 15:1 to about 30:1 and/or at least 30:1.

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain from about 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of PEG-c-DOMG (R-3-[(ω-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In another embodiment the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In one embodiment, the mRNA may be formulated in a lipid nanoparticle such as those described in International Publication No. WO2012170930, the contents of which is herein incorporated by reference in its entirety.

In one embodiment, the mRNA formulation is a nanoparticle which may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In another aspect, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in US Patent Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In one embodiment, the lipid nanoparticle formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of about 20-60% cationic lipid:5-25% neutral lipid:25-55% sterol; 0.5-15% PEG-lipid.

In one embodiment, the formulation includes from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In one embodiment, the formulation includes from about 0.5% to about 15% on a molar basis of the neutral lipid e.g., from about 3 to about 12%, from about 5 to about 10% or about 15%, about 10%, or about 7.5% on a molar basis. Exemplary neutral lipids include, but are not limited to, DSPC, POPC, DPPC, DOPE and SM. In one embodiment, the formulation includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In one embodiment, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis. In one embodiment, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Exemplary PEG-modified lipids include, but are not limited to, PEG-distearoyl glycerol (PEG-DMG) (also referred herein as PEG-C14 or C14-PEG), PEG-cDMA (further discussed in Reyes et al. J. Controlled Release, 107, 276-287 (2005), herein incorporated by reference in its entirety).

In one embodiment, the formulations of the inventions include 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In one embodiment, the formulations of the inventions include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In one embodiment, the formulations of the inventions include 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In one embodiment, the formulations of the inventions include about 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.5% of the neutral lipid, about 31% of the sterol, and about 1.5% of the PEG or PEG-modified lipid on a molar basis.

In one embodiment, the formulations of the inventions include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 38.5% of the sterol, and about 1.5% of the PEG or PEG-modified lipid on a molar basis.

In one embodiment, the formulations of the inventions include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 35% of the sterol, about 4.5% or about 5% of the PEG or PEG-modified lipid, and about 0.5% of the targeting lipid on a molar basis.

In one embodiment, the formulations of the inventions include about 40% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 15% of the neutral lipid, about 40% of the sterol, and about 5% of the PEG or PEG-modified lipid on a molar basis.

In one embodiment, the formulations of the inventions include about 57.2% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.1% of the neutral lipid, about 34.3% of the sterol, and about 1.4% of the PEG or PEG-modified lipid on a molar basis.

In one embodiment, the formulations of the inventions include about 57.5% of a cationic lipid selected from the PEG lipid is PEG-cDMA (PEG-cDMA is further discussed in Reyes et al. (J. Controlled Release, 107, 276-287 (2005), the contents of which are herein incorporated by reference in its entirety), about 7.5% of the neutral lipid, about 31.5% of the sterol, and about 3.5% of the PEG or PEG-modified lipid on a molar basis.

In preferred embodiments, lipid nanoparticle formulation consists essentially of a lipid mixture in molar ratios of about 20-70% cationic lipid: 5-45% neutral lipid: 20-55% cholesterol: 0.5-15% PEG-modified lipid; more preferably in a molar ratio of about 20-60% cationic lipid: 5-25% neutral lipid: 25-55% cholesterol: 0.5-15% PEG-modified lipid.

In particular embodiments, the molar lipid ratio is approximately 50/10/38.5/1.5 mol % cationic lipid/neutral lipid, e.g., DSPC/Chol/PEG-modified lipid, e.g., PEG-DMG, PEG-DSG or PEG-DPG, 57.2/7.1134.3/1.4 mol % cationic lipid/neutral lipid, e.g., DPPC/Chol/PEG-modified lipid, e.g., PEG-cDMA, 40/15/40/5 mol % cationic lipid/neutral lipid, e.g., DSPC/Chol/PEG-modified lipid, e.g., PEG-DMG, 50/10/35/4.5/0.5 mol % cationic lipid/neutral lipid, e.g., DSPC/Chol/PEG-modified lipid, e.g., PEG-DSG, 50/10/35/5 cationic lipid/neutral lipid, e.g., DSPC/Chol/PEG-modified lipid, e.g., PEG-DMG, 40/10/40/10 mol % cationic lipid/neutral lipid, e.g., DSPC/Chol/PEG-modified lipid, e.g., PEG-DMG or PEG-cDMA, 35/15/40/10 mol % cationic lipid/neutral lipid, e.g., DSPC/Chol/PEG-modified lipid, e.g., PEG-DMG or PEG-cDMA or 52/13/30/5 mol % cationic lipid/neutral lipid, e.g., DSPC/Chol/PEG-modified lipid, e.g., PEG-DMG or PEG-cDMA.

Exemplary lipid nanoparticle compositions and methods of making same are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578, herein incorporated by reference in its entirety.

In one embodiment, the lipid nanoparticle formulations described herein may comprise a cationic lipid, a PEG lipid and a structural lipid and optionally comprise a non-cationic lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In one embodiment, the cationic lipid may be any cationic lipid described herein such as, but not limited to, DLin-KC2-DMA, DLin-MC3-DMA and L319.

In one embodiment, the lipid nanoparticle formulations described herein may be 4 component lipid nanoparticles. The lipid nanoparticle may comprise a cationic lipid, a non-cationic lipid, a PEG lipid and a structural lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In one embodiment, the cationic lipid may be any cationic lipid described herein such as, but not limited to, DLin-KC2-DMA, DLin-MC3-DMA and L319.

In one embodiment, the lipid nanoparticle formulations described herein may comprise a cationic lipid, a non-cationic lipid, a PEG lipid and a structural lipid. As a non-limiting example, the lipid nanoparticle comprise about 50% of the cationic lipid DLin-KC2-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DMG and about 38.5% of the structural lipid cholesterol. As yet another non-limiting example, the lipid nanoparticle comprise about 55% of the cationic lipid L319, about 10% of the non-cationic lipid DSPC, about 2.5% of the PEG lipid PEG-DMG and about 32.5% of the structural lipid cholesterol.

In one embodiment, the cationic lipid may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865, WO2008103276, WO2013086373 and WO2013086354, U.S. Pat. Nos. 7,893,302, 7,404,969, 8,283,333, and 8,466,122 and US Patent Publication Nos. US20100036115, US20120202871, US20130064894, US20130129785, US20130150625, US20130178541 and US20130225836; the contents of each of which are herein incorporated by reference in their entirety. In another embodiment, the cationic lipid may be selected from, but not limited to, formula A described in International Publication Nos. WO2012040184, WO2011153120, WO2011149733, WO2011090965, WO2011043913, WO2011022460, WO2012061259, WO2012054365, WO2012044638 and WO2013116126 or US Patent Publication Nos. US20130178541 and US20130225836; the contents of each of which is herein incorporated by reference in their entirety. In yet another embodiment, the cationic lipid may be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula I-VI of US Patent Publication No. US20100036115, formula I of US Patent Publication No US20130123338; each of which is herein incorporated by reference in their entirety. As a non-limiting example, the cationic lipid may be selected from (20Z,23Z) N,N-dimethylnonacosa-20,23-dien-10-amine, (17Z,20Z)—N,N-dimemylhexacosa-17,20-dien-9-amine, (1Z,19Z)—N5N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-diLen-6-amine, (15Z,18Z)—N,N-dimethyltetracosa-15,18-dien-7-amine, (18Z,21Z)—N,N-dimethylheptacosa-18,21-dien-10-amine, (15Z,18Z) N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21 Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21 Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)N-ethyl-N-methylnonacosa-20,23-dien-10-amine, 1-[(11Z,14Z)-1-nonylicosa-11,14-dien-1-yl] pyrrolidine, (20Z)—N,N-dimethylheptacos-20-en-1 0-amine, (15Z)—N,N-dimethyl eptacos-15-en-1 0-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-1 0-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl] eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine,N,N-dimethyl-1-[(1S,2S)-2-{[(1R,2R)-2-pentylcycIopropyl]methyl}cyclopropyl]no nadecan-10-amine,N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S) N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)—N,N-dimethylnonacosa-11,20,2-trien-10-amine or a pharmaceutically acceptable salt or stereoisomer thereof.

In one embodiment, the lipid may be a cleavable lipid such as those described in International Publication No. WO2012170889, herein incorporated by reference in its entirety.

In another embodiment, the lipid may be a cationic lipid such as, but not limited to, Formula (I) of U.S. Patent Application No. US20130064894, the contents of which are herein incorporated by reference in its entirety.

In one embodiment, the cationic lipid may be synthesized by methods known in the art and/or as described in International Publication Nos. WO2012040184, WO2011153120, WO2011149733, WO2011090965, WO2011043913, WO2011022460, WO2012061259, WO2012054365, WO2012044638, WO2010080724, WO201021865, WO2013086373 and WO2013086354; the contents of each of which are herein incorporated by reference in their entirety.

In another embodiment, the cationic lipid may be a trialkyl cationic lipid. Non-limiting examples of trialkyl cationic lipids and methods of making and using the trialkyl cationic lipids are described in International Patent Publication No. WO2013126803, the contents of which are herein incorporated by reference in its entirety.

In one embodiment, the lipid nanoparticle formulations may contain PEG-c-DOMG at 3% lipid molar ratio. In another embodiment, the LNP formulations may contain PEG-c-DOMG at 1.5% lipid molar ratio.

In one embodiment, the pharmaceutical compositions may include at least one of the PEGylated lipids described in International Publication No. WO2012099755, the contents of which is herein incorporated by reference in its entirety.

In one embodiment, the lipid nanoparticle formulation may contain PEG-DMG 2000 (1,2-dimyristoyl-sn-glycero-3-phophoethanolamine-N-[methoxy(polyethylene glycol)-2000). In one embodiment, the LNP formulation may contain PEG-DMG 2000, a cationic lipid known in the art and at least one other component. In another embodiment, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48 (see e.g., Geall et al., Nonviral delivery of self-amplifying RNA vaccines, PNAS 2012; PMID: 22908294, herein incorporated by reference in its entirety).

In one embodiment, the lipid nanoparticle formulation may be formulated by the methods described in International Publication Nos. WO2011127255 or WO2008103276, the contents of each of which is herein incorporated by reference in their entirety. As a non-limiting example, the mRNA described herein may be encapsulated in LNP formulations as described in WO2011127255 and/or WO2008103276; each of which is herein incorporated by reference in their entirety.

In one embodiment, the mRNA described herein may be formulated in a nanoparticle to be delivered by a parenteral route as described in U.S. Pub. No. US20120207845; the contents of which are herein incorporated by reference in its entirety.

In one embodiment, the mRNA may be formulated in a lipid nanoparticle made by the methods described in US Patent Publication No. US20130156845 or International Publication Nos. WO2013093648 or WO2012024526, each of which is herein incorporated by reference in its entirety.

The lipid nanoparticles described herein may be made in a sterile environment by the system and/or methods described in US Patent Publication No. US20130164400, herein incorporated by reference in its entirety.

In one embodiment, the mRNA may be formulated in a nanoparticle such as a nucleic acid-lipid particle described in U.S. Pat. No. 8,492,359, the contents of which are herein incorporated by reference in its entirety. As a non-limiting example, the lipid particle may comprise one or more active agents or therapeutic agents; one or more cationic lipids comprising from about 50 mol % to about 85 mol % of the total lipid present in the particle; one or more non-cationic lipids comprising from about 13 mol % to about 49.5 mol % of the total lipid present in the particle; and one or more conjugated lipids that inhibit aggregation of particles comprising from about 0.5 mol % to about 2 mol % of the total lipid present in the particle. The nucleic acid in the nanoparticle may be the polynucleotides described herein and/or are known in the art.

In one embodiment, the lipid nanoparticle formulation may be formulated by the methods described in International Publication Nos. WO2011127255 or WO2008103276, the contents of each of which are herein incorporated by reference in their entirety. As a non-limiting example, modified RNA polynucleotide described herein may be encapsulated in lipid nanoparticle formulations as described in WO2011127255 and/or WO2008103276; the contents of each of which are herein incorporated by reference in their entirety.

In one embodiment, lipid nanoparticle formulations described herein may comprise a polycationic composition. As a non-limiting example, the polycationic composition may be selected from formula 1-60 of US Patent Publication No. US20050222064; the content of which is herein incorporated by reference in its entirety. In another embodiment, the LNP formulations comprising a polycationic composition may be used for the delivery of the modified RNA described herein in vivo and/or in vitro.

In one embodiment, the LNP formulations described herein may additionally comprise a permeability enhancer molecule. Non-limiting permeability enhancer molecules are described in US Patent Publication No. US20050222064; the content of which is herein incorporated by reference in its entirety.

In one embodiment, the mRNA pharmaceutical compositions may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer as described in Landen et al. Cancer Biology & Therapy 2006 5(12):1708-1713, herein incorporated by reference in its entirety) and hyaluronan-coated liposomes (Quiet Therapeutics, Israel).

In one embodiment, the mRNA may be formulated in a lyophilized gel-phase liposomal composition as described in US Publication No. US2012060293, herein incorporated by reference in its entirety.

The nanoparticle formulations may comprise a phosphate conjugate. The phosphate conjugate may increase in vivo circulation times and/or increase the targeted delivery of the nanoparticle. Phosphate conjugates for use with the present invention may be made by the methods described in International Application No. WO2013033438 or US Patent Publication No. US20130196948, the contents of each of which are herein incorporated by reference in its entirety. As a non-limiting example, the phosphate conjugates may include a compound of any one of the formulas described in International Application No. WO2013033438, herein incorporated by reference in its entirety.

The nanoparticle formulation may comprise a polymer conjugate. The polymer conjugate may be a water soluble conjugate. The polymer conjugate may have a structure as described in U.S. Patent Publication No. US20130059360, the contents of which are herein incorporated by reference in its entirety. In one aspect, polymer conjugates with the polynucleotides of the present invention may be made using the methods and/or segmented polymeric reagents described in U.S. Patent Publication No. US20130072709, herein incorporated by reference in its entirety. In another aspect, the polymer conjugate may have pendant side groups comprising ring moieties such as, but not limited to, the polymer conjugates described in U.S. Patent Publication No. US20130196948, the contents of which is herein incorporated by reference in its entirety.

In another embodiment, pharmaceutical compositions comprising the polynucleotides of the present invention and a conjugate which may have a degradable linkage. Non-limiting examples of conjugates include an aromatic moiety comprising an ionizable hydrogen atom, a spacer moiety, and a water-soluble polymer. As a non-limiting example, pharmaceutical compositions comprising a conjugate with a degradable linkage and methods for delivering such pharmaceutical compositions are described in US Patent Publication No. US20130184443, the contents of which are herein incorporated by reference in its entirety.

The nanoparticle formulations may be a carbohydrate nanoparticle comprising a carbohydrate carrier and an mRNA. As a non-limiting example, the carbohydrate carrier may include, but is not limited to, an anhydride-modified phytoglycogen or glycogen-type material, phytoglycogen octenyl succinate, phytoglycogen beta-dextrin, anhydride-modified phytoglycogen beta-dextrin. (See e.g., International Publication No. WO2012109121; the contents of which are herein incorporated by reference in its entirety).

Nanoparticle formulations of the present invention may be coated with a surfactant or polymer in order to improve the delivery of the particle. In one embodiment, the nanoparticle may be coated with a hydrophilic coating such as, but not limited to, PEG coatings and/or coatings that have a neutral surface charge. The hydrophilic coatings may help to deliver nanoparticles with larger payloads such as, but not limited to, the polynucleotides within the central nervous system. As a non-limiting example nanoparticles comprising a hydrophilic coating and methods of making such nanoparticles are described in U.S. Patent Publication No. US20130183244, the contents of which are herein incorporated by reference in its entirety.

In one embodiment, the lipid nanoparticles of the present invention may be hydrophilic polymer particles. Non-limiting examples of hydrophilic polymer particles and methods of making hydrophilic polymer particles are described in U.S. Patent Publication No. US20130210991, the contents of which are herein incorporated by reference in its entirety.

In another embodiment, the lipid nanoparticles of the present invention may be hydrophobic polymer particles.

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In one embodiment, the internal ester linkage may be located on either side of the saturated carbon.

Lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosal tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104(5):1482-487; Lai et al. Adv Drug Deliv Rev. 2009 61(2): 158-171, herein incorporated by reference in their entirety). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Publication No. WO2013110028, the contents of each of which are herein incorporated by reference in its entirety.

The lipid nanoparticle engineered to penetrate mucus may comprise a polymeric material (i.e. a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyleneimines, polyisocyanates, polyacrylates, polymethacrylates, polyacrylonitriles, and polyarylates. The polymeric material may be biodegradable and/or biocompatible. Non-limiting examples of biocompatible polymers are described in International Publication No. WO2013116804, the contents of which are herein incorporated by reference in its entirety. The polymeric material may additionally be irradiated. As a non-limiting example, the polymeric material may be gamma irradiated (See e.g., International Publication No. WO201282165, herein incorporated by reference in its entirety). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), PEG-PLGA-PEG and trimethylene carbonate, polyvinylpyrrolidone. The lipid nanoparticle may be coated or associated with a co-polymer such as, but not limited to, a block co-polymer (such as a branched polyether-polyamide block copolymer described in International Publication No. WO2013012476, herein incorporated by reference in its entirety), and (poly(ethylene glycol))-(poly(propylene oxide))-(poly(ethylene glycol)) triblock copolymer (see e.g., US Publication 20120121718 and US Publication No. 20100003337 and U.S. Pat. No. 8,263,665; each of which is herein incorporated by reference in their entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:2597-2600, herein incorporated by reference in its entirety). A non-limiting scalable method to produce nanoparticles which can penetrate human mucus is described by Xu et al. (J Control Release 2013, 170(2):279-86, herein incorporated by reference in its entirety).

The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

The lipid nanoparticle engineered to penetrate mucus may include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 dornase alfa, neltenexine, erdosteine) and various DNases including rhDNase. The surface altering agent may be embedded or enmeshed in the particle's surface or disposed (e.g., by coating, adsorption, covalent linkage, or other process) on the surface of the lipid nanoparticle. (see e.g., U.S. Publication Nos. 20100215580, US20080166414, and US20130164343; the contents of each of which is herein incorporated by reference in their entirety).

In one embodiment, the mucus penetrating lipid nanoparticles may comprise at least one polynucleotide described herein. The polynucleotide may be encapsulated in the lipid nanoparticle and/or disposed on the surface of the particle. The polynucleotide may be covalently coupled to the lipid nanoparticle. Formulations of mucus penetrating lipid nanoparticles may comprise a plurality of nanoparticles. Further, the formulations may contain particles which may interact with the mucus and alter the structural and/or adhesive properties of the surrounding mucus to decrease mucoadhesion which may increase the delivery of the mucus penetrating lipid nanoparticles to the mucosal tissue.

In another embodiment, the mucus penetrating lipid nanoparticles may be a hypotonic formulation comprising a mucosal penetration enhancing coating. The formulation may be hypotonic for the epithelium to which it is being delivered. Non-limiting examples of hypotonic formulations may be found in International Patent Publication No. WO2013110028, the contents of which are herein incorporated by reference in its entirety.

In one embodiment, in order to enhance the delivery through the mucosal barrier the polynucleotide formulation may comprise or be a hypotonic solution, see e.g., Ensign et al. Biomaterials 2013 34(28):6922-9, herein incorporated by reference in its entirety.

In one embodiment, the mRNA is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, Mass.), and polyethyleneimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293 Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132, herein incorporated by reference in their entirety).

In one embodiment such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133, herein incorporated by reference in their entirety). One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18:1357-1364, herein incorporated by reference in its entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133, herein incorporated by reference in its entirety).

In one embodiment, the mRNA is formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In a further embodiment, the lipid nanoparticle may be a self-assembly lipid-polymer nanoparticle (see Zhang et al., ACS Nano, 2008, 2 (8), pp 1696-1702, herein incorporated by reference in its entirety). As a non-limiting example, the SLN may be the SLN described in International Patent Publication No. WO2013105101, the contents of which are herein incorporated by reference in its entirety. As another non-limiting example, the SLN may be made by the methods or processes described in International Patent Publication No. WO2013105101, the contents of which are herein incorporated by reference in its entirety.

Liposomes, lipoplexes, or lipid nanoparticles may be used to improve the efficacy of polynucleotides directed protein production as these formulations may be able to increase cell transfection by the mRNA; and/or increase the translation of encoded protein. One such example involves the use of lipid encapsulation to enable the effective systemic delivery of polyplex plasmid DNA (Heyes et al., Mol Ther. 2007 15:713-720; herein incorporated by reference in its entirety). The liposomes, lipoplexes, or lipid nanoparticles may also be used to increase the stability of the polynucleotide.

In one embodiment, the mRNA of the present invention can be formulated for controlled release and/or targeted delivery. As used herein, “controlled release” refers to a pharmaceutical composition or compound release profile that conforms to a particular pattern of release to effect a therapeutic outcome. In one embodiment, the polynucleotide may be encapsulated into a delivery agent described herein and/or known in the art for controlled release and/or targeted delivery. As used herein, the term “encapsulate” means to enclose, surround or encase. As it relates to the formulation of the compounds of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the invention may be enclosed, surrounded or encased within the delivery agent. “Partially encapsulated” means that less than 10, 10, 20, 30, 40 50 or less of the pharmaceutical composition or compound of the invention may be enclosed, surrounded or encased within the delivery agent. Advantageously, encapsulation may be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the delivery agent.

In one embodiment, the controlled release formulation may include, but is not limited to, tri-block co-polymers. As a non-limiting example, the formulation may include two different types of tri-block co-polymers (International Publication Nos. WO2012131104 and WO2012131106; the contents of each of which is herein incorporated by reference in its entirety).

In another embodiment, the mRNA may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

In another embodiment, the lipid nanoparticle may be encapsulated into any polymer known in the art which may form a gel when injected into a subject. As another non-limiting example, the lipid nanoparticle may be encapsulated into a polymer matrix which may be biodegradable.

In one embodiment, the mRNA formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In one embodiment, the mRNA controlled release and/or targeted delivery formulation may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In one embodiment, the mRNA controlled release and/or targeted delivery formulation comprising at least one polynucleotide may comprise at least one PEG and/or PEG related polymer derivatives as described in U.S. Pat. No. 8,404,222, herein incorporated by reference in its entirety.

In another embodiment, the mRNA controlled release delivery formulation comprising at least one polynucleotide may be the controlled release polymer system described in U.S. Patent Publication No. US20130130348, herein incorporated by reference in its entirety.

In one embodiment, the mRNA of the present invention may be encapsulated in a therapeutic nanoparticle, referred to herein as “therapeutic nanoparticles.” Therapeutic nanoparticles may be formulated by methods described herein and known in the art such as, but not limited to, International Publication Nos. WO2010005740, WO2010030763, WO2010005721, WO2010005723, WO2012054923, US Patent Publication Nos. US20110262491, US20100104645, US20100087337, US20100068285, US20110274759, US20100068286, US20120288541, US20130123351 and US20130230567 and U.S. Pat. Nos. 8,206,747, 8,293,276, 8,318,208 and 8,318,211; the contents of each of which are herein incorporated by reference in their entirety. In another embodiment, therapeutic polymer nanoparticles may be identified by the methods described in U.S. Publication No. US20120140790, the contents of which is herein incorporated by reference in its entirety.

In one embodiment, the therapeutic nanoparticles may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the polynucleotides of the present invention (see International Pub No. 2010075072 and US Pub No. US20100216804, US20110217377 and US20120201859, each of which is herein incorporated by reference in their entirety). In another non-limiting example, the sustained release formulation may comprise agents which permit persistent bioavailability such as, but not limited to, crystals, macromolecular gels and/or particulate suspensions (see US Patent Publication No US20130150295, the contents of which is herein incorporated by reference in its entirety).

In one embodiment, the therapeutic nanoparticles may be formulated to be target specific. As a non-limiting example, the therapeutic nanoparticles may include a corticosteroid (see International Publication No. WO2011084518; herein incorporated by reference in its entirety). As a non-limiting example, the therapeutic nanoparticles may be formulated in nanoparticles described in International Publication Nos. WO2008121949, WO2010005726, WO2010005725, WO2011084521 and US Publication Nos. US20100069426, US20120004293 and US20100104655, each of which is herein incorporated by reference in their entirety.

In one embodiment, the nanoparticles of the present invention may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysinc, poly(ethylene imine), poly(serine ester), poly(L-lactide-co-L-lysine), poly(4-hydroxy-L-proline ester) or combinations thereof.

In one embodiment, the therapeutic nanoparticle comprises a diblock copolymer. In one embodiment, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysinc, poly(ethylene imine), poly(serine ester), poly(L-lactide-co-L-lysine), poly(4-hydroxy-L-proline ester) or combinations thereof. In yet another embodiment, the diblock copolymer may be a high-X diblock copolymer such as those described in International Patent Publication No. WO2013120052, the contents of which are herein incorporated by reference in its entirety.

As a non-limiting example the therapeutic nanoparticle comprises a PLGA-PEG block copolymer (see U.S. Publication No. US20120004293 and U.S. Pat. No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968 and International Publication No. WO2012166923, the contents of each of which are herein incorporated by reference in its entirety). In yet another non-limiting example, the therapeutic nanoparticle is a stealth nanoparticle or a target-specific stealth nanoparticle as described in US Patent Publication No. US20130172406, the contents of which are herein incorporated by reference in its entirety.

In one embodiment, the therapeutic nanoparticle may comprise a multiblock copolymer (See e.g., U.S. Pat. Nos. 8,263,665 and 8,287,910 and US Patent Publication No. US20130195987; the contents of each of which are herein incorporated by reference in its entirety).

In yet another non-limiting example, the lipid nanoparticle comprises the block copolymer PEG-PLGA-PEG (see e.g., the thermosensitive hydrogel (PEG-PLGA-PEG) was used as a TGF-betal gene delivery vehicle in Lee et al. Thermosensitive Hydrogel as a Tgf-β1 Gene Delivery Vehicle Enhances Diabetic Wound Healing. Pharmaceutical Research, 2003 20(12): 1995-2000; as a controlled gene delivery system in Li et al. Controlled Gene Delivery System Based on Thermosensitive Biodegradable Hydrogel. Pharmaceutical Research 2003 20(6):884-888; and Chang et al., Non-ionic amphiphilic biodegradable PEG-PLGA-PEG copolymer enhances gene delivery efficiency in rat skeletal muscle. J Controlled Release. 2007 118:245-253, herein incorporated by reference in their entirety). The mRNA of the present invention may be formulated in lipid nanoparticles comprising the PEG-PLGA-PEG block copolymer.

In one embodiment, the therapeutic nanoparticle may comprise a multiblock copolymer (See e.g., U.S. Pat. Nos. 8,263,665 and 8,287,910 and U.S. Patent Publication No. US20130195987; the contents of each of which are herein incorporated by reference in its entirety).

In one embodiment, the block copolymers described herein may be included in a polyion complex comprising a non-polymeric micelle and the block copolymer. (See e.g., U.S. Publication No. 20120076836; herein incorporated by reference in its entirety).

In one embodiment, the therapeutic nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the therapeutic nanoparticles may comprise at least one poly(vinyl ester) polymer. The poly(vinyl ester) polymer may be a copolymer such as a random copolymer. As a non-limiting example, the random copolymer may have a structure such as those described in International Publication No. WO2013032829 or US Patent Publication No. US20130121954, the contents of which are herein incorporated by reference in its entirety. In one aspect, the poly(vinyl ester) polymers may be conjugated to the polynucleotides described herein. In another aspect, the poly(vinyl ester) polymer which may be used in the present invention may be those described in, herein incorporated by reference in its entirety.

In one embodiment, the therapeutic nanoparticle may comprise at least one diblock copolymer. The diblock copolymer may be, but it not limited to, a poly(lactic) acid-poly(ethylene)glycol copolymer (see e.g., International Patent Publication No. WO2013044219; herein incorporated by reference in its entirety).

In one embodiment, the therapeutic nanoparticles may comprise at least one cationic polymer described herein and/or known in the art.

In one embodiment, the therapeutic nanoparticles may comprise at least one amine-containing polymer such as, but not limited to polylysine, polyethylene imine, poly(amidoamine) dendrimers, poly(beta-amino esters) (see e.g., U.S. Pat. No. 8,287,849; herein incorporated by reference in its entirety) and combinations thereof.

In another embodiment, the nanoparticles described herein may comprise an amine cationic lipid such as those described in International Patent Application No. WO2013059496, the contents of which are herein incorporated by reference in its entirety. In one aspect the cationic lipids may have an amino-amine or an amino-amide moiety.

In one embodiment, the therapeutic nanoparticles may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In one embodiment, the therapeutic nanoparticles may be formulated using the methods described by Podobinski et al in U.S. Pat. No. 8,404,799, the contents of which are herein incorporated by reference in its entirety.

In one embodiment, the mRNA may be encapsulated in, linked to and/or associated with synthetic nanocarriers. Synthetic nanocarriers include, but are not limited to, those described in International Publication Nos. WO2010005740, WO2010030763, WO201213501, WO2012149252, WO2012149255, WO2012149259, WO2012149265, WO2012149268, WO2012149282, WO2012149301, WO2012149393, WO2012149405, WO2012149411, WO2012149454 and WO2013019669, and U.S. Publication Nos. US20110262491, US20100104645, US20100087337 and US20120244222, each of which is herein incorporated by reference in their entirety. The synthetic nanocarriers may be formulated using methods known in the art and/or described herein. As a non-limiting example, the synthetic nanocarriers may be formulated by the methods described in International Publication Nos. WO2010005740, WO2010030763 and WO201213501 and US Publication Nos. US20110262491, US20100104645, US20100087337 and US2012024422, each of which is herein incorporated by reference in their entirety. In another embodiment, the synthetic nanocarrier formulations may be lyophilized by methods described in International Publication No. WO2011072218 and U.S. Pat. No. 8,211,473; the content of each of which is herein incorporated by reference in their entirety. In yet another embodiment, formulations of the present invention, including, but not limited to, synthetic nanocarriers, may be lyophilized or reconstituted by the methods described in U.S. Patent Publication No. US20130230568, the contents of which are herein incorporated by reference in its entirety.

In one embodiment, the synthetic nanocarriers may contain reactive groups to release the polynucleotides described herein (see International Pub. No. WO20120952552 and US Pub No. US20120171229, each of which is herein incorporated by reference in their entirety).

In one embodiment, the synthetic nanocarriers may be formulated for targeted release. In one embodiment, the synthetic nanocarrier is formulated to release the polynucleotides at a specified pH and/or after a desired time interval. As a non-limiting example, the synthetic nanoparticle may be formulated to release the mRNA after 24 hours and/or at a pH of 4.5 (see International Publication Nos. WO2010138193 and WO2010138194 and U.S. Publication Nos. US20110020388 and US20110027217, each of which is herein incorporated by reference in their entireties).

In one embodiment, the synthetic nanocarriers may be formulated for controlled and/or sustained release of the polynucleotides described herein. As a non-limiting example, the synthetic nanocarriers for sustained release may be formulated by methods known in the art, described herein and/or as described in International Publication No. WO2010138192 and U.S. Publication No. 20100303850, each of which is herein incorporated by reference in their entirety.

In one embodiment, the mRNA may be formulated for controlled and/or sustained release wherein the formulation comprises at least one polymer that is a crystalline side chain (CYSC) polymer. CYSC polymers are described in U.S. Pat. No. 8,399,007, herein incorporated by reference in its entirety.

In one embodiment, the synthetic nanocarrier may include at least one adjuvant. As non-limiting example, the adjuvant may comprise dimethyldioctadecylammonium-bromide, dimethyldioctadecylammonium-chloride, dimethyldioctadecylammonium-phosphate or dimethyldioctadecylammonium-acetate (DDA) and an apolar fraction or part of said apolar fraction of a total lipid extract of a mycobacterium (See e.g., U.S. Pat. No. 8,241,610; herein incorporated by reference in its entirety). In another embodiment, the synthetic nanocarrier may comprise at least one polynucleotide and an adjuvant. As a non-limiting example, the synthetic nanocarrier comprising and adjuvant may be formulated by the methods described in International Publication No. WO2011150240 and U.S. Publication No. US20110293700, each of which is herein incorporated by reference in its entirety.

In one embodiment, the mRNA may be encapsulated in, linked to and/or associated with zwitterionic lipids. Non-limiting examples of zwitterionic lipids and methods of using zwitterionic lipids are described in U.S. Patent Publication No. US20130216607, the contents of which are herein incorporated by reference in its entirety. I n one aspect, the zwitterionic lipids may be used in the liposomes and lipid nanoparticles described herein.

In one embodiment, the mRNA may be formulated in colloid nanocarriers as described in U.S. Patent Publication No. US20130197100, the contents of which are herein incorporated by reference in its entirety.

In some embodiments, lipid nanoparticles comprise the lipid KL52 (an amino-lipid disclosed in U.S. Patent Publication No. 2012/0295832 expressly incorporated herein by reference in its entirety). Activity and/or safety (as measured by examining one or more of ALT/AST, white blood cell count and cytokine induction) of lipid nanoparticle administration may be improved by incorporation of such lipids. Lipid nanoparticles comprising KL52 may be administered intravenously and/or in one or more doses. In some embodiments, administration of lipid nanoparticles comprising KL52 results in equal or improved mRNA and/or protein expression as compared to LNPs comprising MC3.

In some embodiments, the mRNA may be delivered using smaller lipid nanoparticles. Such particles may comprise a diameter from below 0.1 um up to 100 nm such as, but not limited to, less than 0.1 um, less than 1.0 um, less than 5 um, less than 10 um, less than 15 um, less than 20 um, less than 25 um, less than 30 um, less than 35 um, less than 40 um, less than 50 um, less than 55 um, less than 60 um, less than 65 um, less than 70 um, less than 75 um, less than 80 um, less than 85 um, less than 90 um, less than 95 um, less than 100 um, less than 125 um, less than 150 um, less than 175 um, less than 200 um, less than 225 um, less than 250 um, less than 275 um, less than 300 um, less than 325 um, less than 350 um, less than 375 um, less than 400 um, less than 425 um, less than 450 um, less than 475 um, less than 500 um, less than 525 um, less than 550 um, less than 575 um, less than 600 um, less than 625 um, less than 650 um, less than 675 um, less than 700 um, less than 725 um, less than 750 um, less than 775 um, less than 800 um, less than 825 um, less than 850 um, less than 875 um, less than 900 um, less than 925 um, less than 950 um, less than 975 um,

In another embodiment, the mRNA may be delivered using smaller lipid nanoparticles which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nM, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm.

In some embodiments, such lipid nanoparticles are synthesized using methods comprising microfluidic mixers. Exemplary microfluidic mixers may include, but are not limited to a slit interdigitial micromixer including, but not limited to those manufactured by Microinnova (Allerheiligen bei Wildon, Austria) and/or a staggered herringbone micromixer (SHM) (Zhigaltsev, I. V. et al., Bottom-up design and synthesis of limit size lipid nanoparticle systems with aqueous and triglyceride cores using millisecond microfluidic mixing have been published (Langmuir. 2012. 28:3633-40; Belliveau, N. M. et al., Microfluidic synthesis of highly potent limit-size lipid nanoparticles for in vivo delivery of siRNA. Molecular Therapy-Nucleic Acids. 2012. 1:e37; Chen, D. et al., Rapid discovery of potent siRNA-containing lipid nanoparticles enabled by controlled microfluidic formulation. J Am Chem Soc. 2012. 134(16):6948-51). In some embodiments, methods of lipid nanoparticle generation comprising SHM, further comprise the mixing of at least two input streams wherein mixing occurs by microstructure-induced chaotic advection (MICA). According to this method, fluid streams flow through channels present in a herringbone pattern causing rotational flow and folding the fluids around each other. This method may also comprise a surface for fluid mixing wherein the surface changes orientations during fluid cycling. Methods of generating lipid nanoparticles using SHM include those disclosed in U.S. Patent Publication Nos. 2004/0262223 and 2012/0276209, each of which is expressly incorporated herein by reference in their entirety.

In one embodiment, the mRNA of the present invention may be formulated in lipid nanoparticles created using a micromixer such as, but not limited to, a Slit Interdigital Microstructured Mixer (SIMM-V2) or a Standard Slit Interdigital Micro Mixer (SSIMM) or Caterpillar (CPMM) or Impinging-jet (IJMM) from the Institut für Mikrotechnik Mainz GmbH, Mainz Germany).

In one embodiment, the mRNA of the present invention may be formulated in lipid nanoparticles created using microfluidic technology (see Whitesides, George M. The Origins and the Future of Microfluidics. Nature, 2006 442: 368-373; and Abraham et al. Chaotic Mixer for Microchannels. Science, 2002 295: 647-651, herein incorporated by reference in their entirety). As a non-limiting example, controlled microfluidic formulation includes a passive method for mixing streams of steady pressure-driven flows in micro channels at a low Reynolds number (See e.g., Abraham et al. Chaotic Mixer for Microchannels. Science, 2002 295: 647-651, herein incorporated by reference in its entirety).

In one embodiment, the mRNA of the present invention may be formulated in lipid nanoparticles created using a micromixer chip such as, but not limited to, those from Harvard Apparatus (Holliston, Mass.) or Dolomite Microfluidics (Royston, UK). A micromixer chip can be used for rapid mixing of two or more fluid streams with a split and recombine mechanism.

In one embodiment, the mRNA of the invention may be formulated for delivery using the drug encapsulating microspheres described in International Patent Publication No. WO2013063468 or U.S. Pat. No. 8,440,614, each of which is herein incorporated by reference in its entirety. The microspheres may comprise a compound of the formula (I), (II), (III), (IV), (V) or (VI) as described in International Patent Publication No. WO2013063468, the contents of which are herein incorporated by reference in its entirety. In another aspect, the amino acid, peptide, polypeptide, and lipids (APPL) are useful in delivering the mRNA of the invention to cells as described in International Patent Publication No. WO2013063468, the contents of which is herein incorporated by reference in its entirety.

In one embodiment, the mRNA of the invention may be formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the lipid nanoparticles may have a diameter from about 10 to 500 nm.

In one embodiment, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In one aspect, the lipid nanoparticle may be a limit size lipid nanoparticle described in International Patent Publication No. WO2013059922, the contents of which are herein incorporated by reference in its entirety. The limit size lipid nanoparticle may comprise a lipid bilayer surrounding an aqueous core or a hydrophobic core; where the lipid bilayer may comprise a phospholipid such as, but not limited to, diacylphosphatidylcholine, a diacylphosphatidylethanolamine, a ceramide, a sphingomyelin, a dihydrosphingomyelin, a cephalin, a cerebroside, a C8-C20 fatty acid diacylphophatidylcholine, and 1-palmitoyl-2-oleoyl phosphatidylcholine (POPC). In another aspect the limit size lipid nanoparticle may comprise a polyethylene glycol-lipid such as, but not limited to, DLPE-PEG, DMPE-PEG, DPPC-PEG and DSPE-PEG.

In one embodiment, the mRNA may be delivered, localized and/or concentrated in a specific location using the delivery methods described in International Patent Publication No. WO2013063530, the contents of which are herein incorporated by reference in its entirety. As a non-limiting example, a subject may be administered an empty polymeric particle prior to, simultaneously with or after delivering the mRNA to the subject. The empty polymeric particle undergoes a change in volume once in contact with the subject and becomes lodged, embedded, immobilized or entrapped at a specific location in the subject.

In one embodiment, the mRNA may be formulated in an active substance release system (See e.g., U.S. Patent Publication No. US20130102545, the contents of which is herein incorporated by reference in its entirety). The active substance release system may comprise 1) at least one nanoparticle bonded to an oligonucleotide inhibitor strand which is hybridized with a catalytically active nucleic acid and 2) a compound bonded to at least one substrate molecule bonded to a therapeutically active substance (e.g., polynucleotides described herein), where the therapeutically active substance is released by the cleavage of the substrate molecule by the catalytically active nucleic acid.

In one embodiment, the mRNA may be formulated in a nanoparticle comprising an inner core comprising a non-cellular material and an outer surface comprising a cellular membrane. The cellular membrane may be derived from a cell or a membrane derived from a virus. As a non-limiting example, the nanoparticle may be made by the methods described in International Patent Publication No. WO2013052167, herein incorporated by reference in its entirety. As another non-limiting example, the nanoparticle described in International Patent Publication No. WO2013052167, herein incorporated by reference in its entirety, may be used to deliver the mRNA described herein.

In one embodiment, the mRNA may be formulated in porous nanoparticle-supported lipid bilayers (protocells). Protocells are described in International Patent Publication No. WO2013056132, the contents of which are herein incorporated by reference in its entirety.

In one embodiment, the mRNA described herein may be formulated in polymeric nanoparticles as described in or made by the methods described in U.S. Pat. Nos. 8,420,123 and 8,518,963 and European Patent No. EP2073848B1, the contents of each of which are herein incorporated by reference in their entirety. As a non-limiting example, the polymeric nanoparticle may have a high glass transition temperature such as the nanoparticles described in or nanoparticles made by the methods described in U.S. Pat. No. 8,518,963, the contents of which are herein incorporated by reference in its entirety. As another non-limiting example, the polymer nanoparticle for oral and parenteral formulations may be made by the methods described in European Patent No. EP2073848B1, the contents of which are herein incorporated by reference in its entirety.

In another embodiment, the mRNA described herein may be formulated in nanoparticles used in imaging. The nanoparticles may be liposome nanoparticles such as those described in US Patent Publication No US20130129636, herein incorporated by reference in its entirety. As a non-limiting example, the liposome may comprise gadolinium(III)2-{4,7-bis-carboxymethyl-10-[(N,N-distearylamidomethyl-N amido-methyl]-1,4,7,10-tetra-azacyclododec-1-yl}-acetic acid and a neutral, fully saturated phospholipid component (see e.g., US Patent Publication No US20130129636, the contents of which is herein incorporated by reference in its entirety).

In one embodiment, the nanoparticles which may be used in the present invention are formed by the methods described in U.S. Patent Publication No. US20130130348, the contents of which is herein incorporated by reference in its entirety.

The nanoparticles of the present invention may further include nutrients such as, but not limited to, those which deficiencies can lead to health hazards from anemia to neural tube defects (see e.g., the nanoparticles described in International Patent Publication No. WO2013072929, the contents of which is herein incorporated by reference in its entirety). As a non-limiting example, the nutrient may be iron in the form of ferrous, ferric salts or elemental iron, iodine, folic acid, vitamins or micronutrients.

In one embodiment, the mRNA of the present invention may be formulated in a swellable nanoparticle. The swellable nanoparticle may be, but is not limited to, those described in U.S. Pat. No. 8,440,231, the contents of which is herein incorporated by reference in its entirety. As a non-limiting embodiment, the swellable nanoparticle may be used for delivery of the mRNA of the present invention to the pulmonary system (see e.g., U.S. Pat. No. 8,440,231, the contents of which is herein incorporated by reference in its entirety).

The mRNA of the present invention may be formulated in polyanhydride nanoparticles such as, but not limited to, those described in U.S. Pat. No. 8,449,916, the contents of which is herein incorporated by reference in its entirety.

The nanoparticles and microparticles of the present invention may be geometrically engineered to modulate macrophage and/or the immune response. In one aspect, the geometrically engineered particles may have varied shapes, sizes and/or surface charges in order to incorporated the polynucleotides of the present invention for targeted delivery such as, but not limited to, pulmonary delivery (see e.g., International Publication No. WO2013082111, the contents of which is herein incorporated by reference in its entirety). Other physical features the geometrically engineering particles may have include, but are not limited to, fenestrations, angled arms, asymmetry and surface roughness, charge which can alter the interactions with cells and tissues. As a non-limiting example, nanoparticles of the present invention may be made by the methods described in International Publication No WO2013082111, the contents of which is herein incorporated by reference in its entirety.

In one embodiment, the nanoparticles of the present invention may be water soluble nanoparticles such as, but not limited to, those described in International Publication No. WO2013090601, the contents of which is herein incorporated by reference in its entirety. The nanoparticles may be inorganic nanoparticles which have a compact and zwitterionic ligand in order to exhibit good water solubility. The nanoparticles may also have small hydrodynamic diameters (HD), stability with respect to time, pH, and salinity and a low level of non-specific protein binding.

In one embodiment the nanoparticles of the present invention may be developed by the methods described in U.S. Patent Publication No. US20130172406, the contents of which are herein incorporated by reference in its entirety.

In one embodiment, the nanoparticles of the present invention are stealth nanoparticles or target-specific stealth nanoparticles such as, but not limited to, those described in U.S. Patent Publication No. US20130172406; the contents of which is herein incorporated by reference in its entirety. The nanoparticles of the present invention may be made by the methods described in U.S. Patent Publication No. US20130172406, the contents of which are herein incorporated by reference in its entirety.

In another embodiment, the stealth or target-specific stealth nanoparticles may comprise a polymeric matrix. The polymeric matrix may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates or combinations thereof.

In one embodiment, the nanoparticle may be a nanoparticle-nucleic acid hybrid structure having a high-density nucleic acid layer. As a non-limiting example, the nanoparticle-nucleic acid hybrid structure may made by the methods described in U.S. Patent Publication No. US20130171646, the contents of which are herein incorporated by reference in its entirety. The nanoparticle may comprise a nucleic acid such as, but not limited to, polynucleotides described herein and/or known in the art.

At least one of the nanoparticles of the present invention may be embedded in the core a nanostructure or coated with a low density porous 3-D structure or coating which is capable of carrying or associating with at least one payload within or on the surface of the nanostructure. Non-limiting examples of the nanostructures comprising at least one nanoparticle are described in International Patent Publication No. WO2013123523, the contents of which are herein incorporated by reference in its entirety.

### Polymer and Polymeric Nanoparticle Formulations

The mRNA of the invention can be formulated using natural and/or synthetic polymers. Non-limiting examples of polymers which may be used for delivery include, but are not limited to, DYNAMIC POLYCONJUGATE® (Arrowhead Research Corp., Pasadena, Calif.) formulations from MIRUS® Bio (Madison, Wis.) and Roche Madison (Madison, Wis.), PHASERX™ polymer formulations such as, without limitation, SMARTT POLYMER TECHNOLOGY™ (PHASERX®, Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly(lactic-co-glycolic acid) (PLGA) polymers. RONDEL™ (RNAi/Oligonucleotide Nanoparticle Delivery) polymers (Arrowhead Research Corporation, Pasadena, Calif.) and pH responsive co-block polymers such as, but not limited to, PHASERX® (Seattle, Wash.).

A non-limiting example of chitosan formulation includes a core of positively charged chitosan and an outer portion of negatively charged substrate (U.S. Publication No. 20120258176; herein incorporated by reference in its entirety). Chitosan includes, but is not limited to N-trimethyl chitosan, mono-N-carboxymethyl chitosan (MCC), N-palmitoyl chitosan (NPCS), EDTA-chitosan, low molecular weight chitosan, chitosan derivatives, or combinations thereof.

In one embodiment, the polymers used in the present invention have undergone processing to reduce and/or inhibit the attachment of unwanted substances such as, but not limited to, bacteria, to the surface of the polymer. The polymer may be processed by methods known and/or described in the art and/or described in International Publication No. WO2012150467, herein incorporated by reference in its entirety.

A non-limiting example of PLGA formulations include, but are not limited to, PLGA injectable depots (e.g., ELIGARD® which is formed by dissolving PLGA in 66% N-methyl-2-pyrrolidone (NMP) and the remainder being aqueous solvent and leuprolide. Once injected, the PLGA and leuprolide peptide precipitates into the subcutaneous space).

Many of these polymer approaches have demonstrated efficacy in delivering oligonucleotides in vivo into the cell cytoplasm (reviewed in deFougerolles Hum Gene Ther. 2008 19:125-132). Two polymer approaches that have yielded robust in vivo delivery of nucleic acids, in this case with small interfering RNA (siRNA), are dynamic polyconjugates and cyclodextrin-based nanoparticles (see e.g., U.S. Patent Publication No. US20130156721, herein incorporated by reference in its entirety). The first of these delivery approaches uses dynamic polyconjugates and has been shown in vivo in mice to effectively deliver siRNA and silence endogenous target mRNA in hepatocytes (Rozema et al., Proc Natl Acad Sci USA. 2007 104:12982-12887, herein incorporated by reference in its entirety). This particular approach is a multicomponent polymer system whose key features include a membrane-active polymer to which nucleic acid, in this case siRNA, is covalently coupled via a disulfide bond and where both PEG (for charge masking) and N-acetylgalactosamine (for hepatocyte targeting) groups are linked via pH-sensitive bonds (Rozema et al., Proc Natl Acad Sci USA. 2007 104:12982-12887, herein incorporated by reference in its entirety). On binding to the hepatocyte and entry into the endosome, the polymer complex disassembles in the low-pH environment, with the polymer exposing its positive charge, leading to endosomal escape and cytoplasmic release of the siRNA from the polymer. Through replacement of the N-acetylgalactosamine group with a mannose group, it was shown one could alter targeting from asialoglycoprotein receptor-expressing hepatocytes to sinusoidal endothelium and Kupffer cells. Another polymer approach involves using transferrin-targeted cyclodextrin-containing polycation nanoparticles. These nanoparticles have demonstrated targeted silencing of the EWS-FLI1 gene product in transferrin receptor-expressing Ewing's sarcoma tumor cells (Hu-Lieskovan et al., Cancer Res. 2005 65: 8984-8982, herein incorporated by reference in its entirety) and siRNA formulated in these nanoparticles was well tolerated in non-human primates (Heidel et al., Proc Natl Acad Sci USA 2007 104:5715-21, herein incorporated by reference in its entirety). Both of these delivery strategies incorporate rational approaches using both targeted delivery and endosomal escape mechanisms.

The polymer formulation can permit the sustained or delayed release of polynucleotides. The altered release profile for the polynucleotide can result in, for example, translation of an encoded protein over an extended period of time. The polymer formulation may also be used to increase the stability of the polynucleotide. Biodegradable polymers have been previously used to protect nucleic acids other than polynucleotide from degradation and been shown to result in sustained release of payloads in vivo (Rozema et al., Proc Natl Acad Sci USA. 2007 104:12982-12887; Sullivan et al., Expert Opin Drug Deliv. 2010 7:1433-1446; Convertine et al., Biomacromolecules. 2010 Oct. 1; Chu et al., Acc Chem Res. 2012 Jan. 13; Manganiello et al., Biomaterials. 2012 33:2301-2309; Benoit et al., Biomacromolecules. 2011 12:2708-2714; Singha et al., Nucleic Acid Ther. 2011 2:133-147; deFougerolles Hum Gene Ther. 2008 19:125-132; Schaffert and Wagner, Gene Ther. 2008 16:1131-1138; Chaturvedi et al., Expert Opin Drug Deliv. 2011 8:1455-1468; Davis, Mol Pharm. 2009 6:659-668; Davis, Nature 2010 464:1067-1070, herein incorporated by reference in their entirety).

In one embodiment, the mRNA pharmaceutical compositions may be sustained release formulations. Sustained release formulations may include, but are not limited to, PLGA microspheres, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

As a non-limiting example, the mRNA may be formulated in PLGA microspheres by preparing the PLGA microspheres with tunable release rates (e.g., days and weeks) and encapsulating the modified mRNA in the PLGA microspheres while maintaining the integrity of the modified mRNA during the encapsulation process. EVAc are non-biodegradable, biocompatible polymers which are used extensively in pre-clinical sustained release implant applications (e.g., extended release products Ocusert a pilocarpine ophthalmic insert for glaucoma or progestasert a sustained release progesterone intrauterine device; transdermal delivery systems Testoderm, Duragesic and Selegiline; catheters). Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C. PEG-based surgical sealants comprise two synthetic PEG components mixed in a delivery device which can be prepared in one minute, seals in 3 minutes and is reabsorbed within 30 days. GELSITE® and natural polymers are capable of in-situ gelation at the site of administration. They have been shown to interact with protein and peptide therapeutic candidates through ionic interaction to provide a stabilizing effect.

Polymer formulations can also be selectively targeted through expression of different ligands as exemplified by, but not limited by, folate, transferrin, and N-acetylgalactosamine (GalNAc) (Benoit et al., Biomacromolecules. 2011 12:2708-2714; Rozema et al., Proc Natl Acad Sci USA. 2007 104:12982-12887; Davis, Mol Pharm. 2009 6:659-668; Davis, Nature 2010 464:1067-1070, herein incorporated by reference in their entirety).

The mRNA of the invention may be formulated with or in a polymeric compound. The polymer may include at least one polymer such as, but not limited to, polyethenes, polyethylene glycol (PEG), poly(l-lysine)(PLL), PEG grafted to PLL, cationic lipopolymer, biodegradable cationic lipopolymer, polyethyleneimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof.

As a non-limiting example, the mRNA of the invention may be formulated with the polymeric compound of PEG grafted with PLL as described in U.S. Pat. No. 6,177,274; herein incorporated by reference in its entirety. The formulation may be used for transfecting cells in vitro or for in vivo delivery of polynucleotides. In another example, the polynucleotide may be suspended in a solution or medium with a cationic polymer, in a dry pharmaceutical composition or in a solution that is capable of being dried as described in U.S. Publication Nos. 20090042829 and 20090042825; each of which are herein incorporated by reference in their entireties.

As another non-limiting example. the mRNA of the invention may be formulated with a PLGA-PEG block copolymer (see U.S. Publication No. US20120004293 and U.S. Pat. No. 8,236,330, herein incorporated by reference in their entireties) or PLGA-PEG-PLGA block copolymers (See U.S. Pat. No. 6,004,573, herein incorporated by reference in its entirety). As a non-limiting example, the mRNA of the invention may be formulated with a diblock copolymer of PEG and PLA or PEG and PLGA (see U.S. Pat. No. 8,246,968, herein incorporated by reference in its entirety).

A polyamine derivative may be used to deliver nucleic acids or to treat and/or prevent a disease or to be included in an implantable or injectable device (U.S. Publication No. 20100260817 (now U.S. Pat. No. 8,460,696) the contents of each of which is herein incorporated by reference in its entirety). As a non-limiting example, a pharmaceutical composition may include the mRNA and the polyamine derivative described in U.S. Publication No. 20100260817 (now U.S. Pat. No. 8,460,696; the contents of which are incorporated herein by reference in its entirety. As a non-limiting example, the mRNA of the present invention may be delivered using a polyamide polymer such as, but not limited to, a polymer comprising a 1,3-dipolar addition polymer prepared by combining a carbohydrate diazide monomer with a dilkyne unite comprising oligoamines (U.S. Pat. No. 8,236,280; herein incorporated by reference in its entirety).

The mRNA of the invention may be formulated with at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the mRNA of the present invention may be formulated with at least one polymer and/or derivatives thereof described in International Publication Nos. WO2011115862, WO2012082574 and WO2012068187 and U.S. Publication No. 20120283427, each of which are herein incorporated by reference in their entireties.

In another embodiment, the mRNA of the present invention may be formulated with a polymer of formula Z as described in International Patent Publication No. WO2011115862, herein incorporated by reference in its entirety. In yet another embodiment, the mRNA may be formulated with a polymer of formula Z, Z′ or Z″ as described in International Publication Nos. WO2012082574 or WO2012068187 and U.S. Publication No. 2012028342, each of which are herein incorporated by reference in their entireties. The polymers formulated with the modified RNA of the present invention may be synthesized by the methods described in International Publication Nos. WO2012082574 or WO2012068187, each of which are herein incorporated by reference in their entireties.

The mRNA of the invention may be formulated with at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

Formulations of the mRNA of the invention may include at least one amine-containing polymer such as, but not limited to polylysine, polyethylene imine, poly(amidoamine) dendrimers, poly(amine-co-esters) or combinations thereof. As a non-limiting example, the poly(amine-co-esters) may be the polymers described in and/or made by the methods described in International Publication No. WO2013082529, the contents of which are herein incorporated by reference in its entirety.

For example, the mRNA of the invention may be formulated in a pharmaceutical compound including a poly(alkylene imine), a biodegradable cationic lipopolymer, a biodegradable block copolymer, a biodegradable polymer, or a biodegradable random copolymer, a biodegradable polyester block copolymer, a biodegradable polyester polymer, a biodegradable polyester random copolymer, a linear biodegradable copolymer, PAGA, a biodegradable cross-linked cationic multi-block copolymer or combinations thereof. The biodegradable cationic lipopolymer may be made by methods known in the art and/or described in U.S. Pat. No. 6,696,038, U.S. Publication Nos. 20030073619 and 20040142474 each of which is herein incorporated by reference in their entireties. The poly(alkylene imine) may be made using methods known in the art and/or as described in U.S. Publication No. 20100004315, herein incorporated by reference in its entirety. The biodegradable polymer, biodegradable block copolymer, the biodegradable random copolymer, biodegradable polyester block copolymer, biodegradable polyester polymer, or biodegradable polyester random copolymer may be made using methods known in the art and/or as described in U.S. Pat. Nos. 6,517,869 and 6,267,987, the contents of which are each incorporated herein by reference in their entirety. The linear biodegradable copolymer may be made using methods known in the art and/or as described in U.S. Pat. No. 6,652,886. The PAGA polymer may be made using methods known in the art and/or as described in U.S. Pat. No. 6,217,912 herein incorporated by reference in its entirety. The PAGA polymer may be copolymerized to form a copolymer or block copolymer with polymers such as but not limited to, poly-L-lysine, polyargine, polyornithine, histones, avidin, protamines, polylactides and poly(lactide-co-glycolides). The biodegradable cross-linked cationic multi-block copolymers may be made my methods known in the art and/or as described in U.S. Pat. Nos. 8,057,821, 8,444,992 or U.S. Publication No. 2012009145 each of which are herein incorporated by reference in their entireties. For example, the multi-block copolymers may be synthesized using linear polyethyleneimine (LPEI) blocks which have distinct patterns as compared to branched polyethyleneimines. Further, the composition or pharmaceutical composition may be made by the methods known in the art, described herein, or as described in U.S. Publication No. 20100004315 or U.S. Pat. Nos. 6,267,987 and 6,217,912 each of which are herein incorporated by reference in their entireties.

The mRNA of the invention may be formulated with at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

The mRNA of the invention may be formulated with at least one crosslinkable polyester. Crosslinkable polyesters include those known in the art and described in U.S. Publication No. 20120269761, the contents of which is herein incorporated by reference in its entirety.

The mRNA of the invention may be formulated in or with at least one cyclodextrin polymer. Cyclodextrin polymers and methods of making cyclodextrin polymers include those known in the art and described in U.S. Publication No. 20130184453, the contents of which are herein incorporated by reference in its entirety.

In one embodiment, the mRNA of the invention may be formulated in or with at least one crosslinked cation-binding polymers. Crosslinked cation-binding polymers and methods of making crosslinked cation-binding polymers include those known in the art and described in International Patent Publication Nos. WO2013106072, WO2013106073 and WO2013106086, the contents of each of which are herein incorporated by reference in its entirety.

In one embodiment, the mRNA of the invention may be formulated in or with at least one branched polymer. Branched polymers and methods of making branched polymers include those known in the art and described in International Patent Publication No. WO2013113071, the contents of each of which are herein incorporated by reference in its entirety.

In one embodiment, the mRNA of the invention may be formulated in or with at least PEGylated albumin polymer. PEGylated albumin polymer and methods of making PEGylated albumin polymer include those known in the art and described in U.S. Patent Publication No. US20130231287, the contents of each of which are herein incorporated by reference in its entirety.

In one embodiment, the polymers described herein may be conjugated to a lipid-terminating PEG. As a non-limiting example, PLGA may be conjugated to a lipid-terminating PEG forming PLGA-DSPE-PEG. As another non-limiting example, PEG conjugates for use with the present invention are described in International Publication No. WO2008103276, herein incorporated by reference in its entirety. The polymers may be conjugated using a ligand conjugate such as, but not limited to, the conjugates described in U.S. Pat. No. 8,273,363, herein incorporated by reference in its entirety.

In one embodiment, the mRNA disclosed herein may be mixed with the PEGs or the sodium phosphate/sodium carbonate solution prior to administration.

In another embodiment, polynucleotides encoding the protein of interest may be mixed with the PEGs and also mixed with the sodium phosphate/sodium carbonate solution.

In one embodiment, the mRNA described herein may be conjugated with another compound. Non-limiting examples of conjugates are described in U.S. Pat. Nos. 7,964,578 and 7,833,992, each of which are herein incorporated by reference in their entireties. In another embodiment, the mRNA of the present invention may be conjugated with conjugates of formula 1-122 as described in U.S. Pat. Nos. 7,964,578 and 7,833,992, each of which are herein incorporated by reference in their entireties. The mRNA described herein may be conjugated with a metal such as, but not limited to, gold. (See e.g., Giljohann et al. Journ. Amer. Chem. Soc. 2009 131(6): 2072-2073). In another embodiment, the mRNA described herein may be conjugated and/or encapsulated in gold-nanoparticles. (International Publication No. WO201216269 and U.S. Publication No. 20120302940 and US20130177523; the contents of each of which is herein incorporated by reference in its entirety).

In one embodiment, the polymer formulation of the present invention may be stabilized by contacting the polymer formulation, which may include a cationic carrier, with a cationic lipopolymer which may be covalently linked to cholesterol and polyethylene glycol groups. The polymer formulation may be contacted with a cationic lipopolymer using the methods described in U.S. Publication No. 20090042829 herein incorporated by reference in its entirety. The cationic carrier may include, but is not limited to, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B—[N—(N′,N′-Dimethylaminoethane)-carbamoyl]Cholesterol Hydrochloride (DC-Cholesterol HCl) diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride DODAC) and combinations thereof. As a non-limiting example, the mRNA may be formulated with a cationic lipopolymer such as those described in U.S. Patent Publication No. 20130065942, the contents of which are herein incorporated by reference in its entirety.

The mRNA of the invention may be formulated in a polyplex of one or more polymers (See e.g., U.S. Pat. No. 8,501,478, U.S. Publication Nos. 20120237565, 20120270927 and 20130149783 and International Patent Publication No. WO2013090861; the contents of each of which is herein incorporated by reference in its entirety). As a non-limiting example, the polyplex may be formed using the noval alpha-aminoamidine polymers described in International Publication No. WO2013090861, the contents of which are herein incorporated by reference in its entirety. As another non-limiting example, the polyplex may be formed using the click polymers described in U.S. Pat. No. 8,501,478, the contents of which is herein incorporated by reference in its entirety.

In one embodiment, the polyplex comprises two or more cationic polymers. The cationic polymer may comprise a poly(ethylene imine) (PEI) such as linear PEI. In another embodiment, the polyplex comprises p(TETA/CBA) its PEGylated analog p(TETA/CBA)-g-PEG2k and mixtures thereof (see e.g., US Patent Publication No. US20130149783, the contents of which are herein incorporated by reference in its entirety.

The mRNA of the invention can also be formulated as a nanoparticle using a combination of polymers, lipids, and/or other biodegradable agents, such as, but not limited to, calcium phosphate. Components may be combined in a core-shell, hybrid, and/or layer-by-layer architecture, to allow for fine-tuning of the nanoparticle so to delivery of the polynucleotide, may be enhanced (Wang et al., Nat Mater. 2006 5:791-796; Fuller et al., Biomaterials. 2008 29:1526-1532; DeKoker et al., Adv Drug Deliv Rev. 2011 63:748-761; Endres et al., Biomaterials. 2011 32:7721-7731; Su et al., Mol Pharm. 2011 Jun. 6; 8(3):774-87, herein incorporated by reference in its entirety). As a non-limiting example, the nanoparticle may comprise a plurality of polymers such as, but not limited to hydrophilic-hydrophobic polymers (e.g., PEG-PLGA), hydrophobic polymers (e.g., PEG) and/or hydrophilic polymers (International Publication No. WO20120225129; the contents of which is herein incorporated by reference in its entirety).

As another non-limiting example the nanoparticle comprising hydrophilic polymers for the mRNA may be those described in or made by the methods described in International Patent Publication No. WO2013 119936, the contents of which are herein incorporated by reference in its entirety.

In one embodiment, the biodegradable polymers which may be used in the present invention are poly(ether-anhydride) block copolymers. As a non-limiting example, the biodegradable polymers used herein may be a block copolymer as described in International Patent Publication No. WO2006063249, herein incorporated by reference in its entirety, or made by the methods described in International Patent Publication No. WO2006063249, herein incorporated by reference in its entirety.

In another embodiment, the biodegradable polymers which may be used in the present invention are alkyl and cycloalkyl terminated biodegradable lipids. As a non-limiting example, the alkyl and cycloalkyl terminated biodegradable lipids may be those described in International Publication No. WO2013086322 and/or made by the methods described in International Publication No. WO2013086322; the contents of which are herein incorporated by reference in its entirety.

In yet another embodiment, the biodegradable polymers which may be used in the present invention are cationic lipids having one or more biodegradable group located in a lipid moiety. As a non-limiting example, the biodegradable lipids may be those described in U.S. Patent Publication No. US20130195920, the contents of which are herein incorporated by reference in its entirety.

Biodegradable calcium phosphate nanoparticles in combination with lipids and/or polymers have been shown to deliver polynucleotides in vivo. In one embodiment, a lipid coated calcium phosphate nanoparticle, which may also contain a targeting ligand such as anisamide, may be used to deliver the mRNA of the present invention. For example, to effectively deliver siRNA in a mouse metastatic lung model a lipid coated calcium phosphate nanoparticle was used (Li et al., J Contr Rel. 2010 142: 416-421; Li et al., J Contr Rel. 2012 158:108-114; Yang et al., Mol Ther. 2012 20:609-615, herein incorporated by reference in their entirety). This delivery system combines both a targeted nanoparticle and a component to enhance the endosomal escape, calcium phosphate, in order to improve delivery of the siRNA.

In one embodiment, calcium phosphate with a PEG-polyanion block copolymer may be used to deliver the mRNA (Kazikawa et al., J Contr Rel. 2004 97:345-356; Kazikawa et al., J Contr Rel. 2006 111:368-370, herein incorporated by reference in their entirety).

In one embodiment, a PEG-charge-conversional polymer (Pitella et al., Biomaterials. 2011 32:3106-3114, herein incorporated by reference in their entirety) may be used to form a nanoparticle to deliver the mRNA of the present invention. The PEG-charge-conversional polymer may improve upon the PEG-polyanion block copolymers by being cleaved into a polycation at acidic pH, thus enhancing endosomal escape.

In one embodiment, a polymer used in the present invention may be a pentablock polymer such as, but not limited to, the pentablock polymers described in International Patent Publication No. WO2013055331, herein incorporated by reference in its entirety. As a non-limiting example, the pentablock polymer comprises PGA-PCL-PEG-PCL-PGA, wherein PEG is polyethylene glycol, PCL is poly(E-caprolactone), PGA is poly(glycolic acid), and PLA is poly(lactic acid). As another non-limiting example, the pentablock polymer comprises PEG-PCL-PLA-PCL-PEG, wherein PEG is polyethylene glycol, PCL is poly(E-caprolactone), PGA is poly(glycolic acid), and PLA is poly(lactic acid).

In one embodiment, a polymer which may be used in the present invention comprises at least one diepoxide and at least one aminoglycoside (See e.g., International Patent Publication No. WO2013055971, the contents of which are herein incorporated by reference in its entirety). The diepoxide may be selected from, but is not limited to, 1,4 butanediol diglycidyl ether (1,4 B), 1,4-cyclohexanedimethanol diglycidyl ether (1,4 C), 4-vinylcyclohexene diepoxide (4VCD), ethyleneglycol diglycidyl ether (EDGE), glycerol diglycidyl ether (GDE), neopentylglycol diglycidyl ether (NPDGE), poly(ethyleneglycol) diglycidyl ether (PEGDE), poly(propyleneglycol) diglycidyl ether (PPGDE) and resorcinol diglycidyl ether (RDE). The aminoglycoside may be selected from, but is not limited to, streptomycin, neomycin, framycetin, paromomycin, ribostamycin, kanamycin, amikacin, arbekacin, bekanamycin, dibekacin, tobramycin, spectinomycin, hygromycin, gentamicin, netilmicin, sisomicin, isepamicin, verdamicin, astromicin, and apramycin. As a non-limiting example, the polymers may be made by the methods described in International Patent Publication No. WO2013055971, the contents of which are herein incorporated by reference in its entirety. As another non-limiting example, compositions comprising any of the polymers comprising at least one least one diepoxide and at least one aminoglycoside may be made by the methods described in International Patent Publication No. WO2013055971, the contents of which are herein incorporated by reference in its entirety.

In one embodiment, a polymer which may be used in the present invention may be a cross-linked polymer. As a non-limiting example, the cross-linked polymers may be used to form a particle as described in U.S. Pat. No. 8,414,927, the contents of which are herein incorporated by reference in its entirety. As another non-limiting example, the cross-linked polymer may be obtained by the methods described in U.S. Patent Publication No. US20130172600, the contents of which are herein incorporated by reference in its entirety.

In another embodiment, a polymer which may be used in the present invention may be a cross-linked polymer such as those described in U.S. Pat. No. 8,461,132, the contents of which are herein incorporated by reference in its entirety. As a non-limiting example, the cross-linked polymer may be used in a therapeutic composition for the treatment of a body tissue. The therapeutic composition may be administered to damaged tissue using various methods known in the art and/or described herein such as injection or catheterization.

In one embodiment, a polymer which may be used in the present invention may be a di-alphatic substituted pegylated lipid such as, but not limited to, those described in International Patent Publication No. WO2013049328, the contents of which are herein incorporated by reference in its entirety.

In one embodiment, a block copolymer is PEG-PLGA-PEG (see e.g., the thermosensitive hydrogel (PEG-PLGA-PEG) was used as a TGF-betal gene delivery vehicle in Lee et al. Thermosensitive Hydrogel as a Tgf-01 Gene Delivery Vehicle Enhances Diabetic Wound Healing. Pharmaceutical Research, 2003 20(12): 1995-2000; as a controlled gene delivery system in Li et al. Controlled Gene Delivery System Based on Thermo sensitive Biodegradable Hydrogel. Pharmaceutical Research 2003 20(6):884-888; and Chang et al., Non-ionic amphiphilic biodegradable PEG-PLGA-PEG copolymer enhances gene delivery efficiency in rat skeletal muscle. J Controlled Release. 2007 118:245-253), herein incorporated by reference in their entirety, may be used in the present invention. The present invention may be formulated with PEG-PLGA-PEG for administration such as, but not limited to, intramuscular and subcutaneous administration.

In another embodiment, the PEG-PLGA-PEG block copolymer is used in the present invention to develop a biodegradable sustained release system. In one aspect, the mRNA of the present invention is mixed with the block copolymer prior to administration. In another aspect, the mRNA of the present invention is co-administered with the block copolymer.

In one embodiment, the polymer used in the present invention may be a multi-functional polymer derivative such as, but not limited to, a multi-functional N-maleimidyl polymer derivatives as described in U.S. Pat. No. 8,454,946, the contents of which are herein incorporated by reference in its entirety.

The use of core-shell nanoparticles has additionally focused on a high-throughput approach to synthesize cationic cross-linked nanogel cores and various shells (Siegwart et al., Proc Natl Acad Sci USA. 2011 108:12996-13001, herein incorporated by reference in its entirety). The complexation, delivery, and internalization of the polymeric nanoparticles can be precisely controlled by altering the chemical composition in both the core and shell components of the nanoparticle. For example, the core-shell nanoparticles may efficiently deliver siRNA to mouse hepatocytes after they covalently attach cholesterol to the nanoparticle.

In one embodiment, a hollow lipid core comprising a middle PLGA layer and an outer neutral lipid layer containing PEG may be used to delivery of the mRNA of the present invention. As a non-limiting example, in mice bearing a luciferease-expressing tumor, it was determined that the lipid-polymer-lipid hybrid nanoparticle significantly suppressed luciferase expression, as compared to a conventional lipoplex (Shi et al, Angew Chem Int Ed. 2011 50:7027-7031; herein incorporated by reference in its entirety).

In one embodiment, the lipid nanoparticles may comprise a core of the mRNA disclosed herein and a polymer shell. The polymer shell may be any of the polymers described herein and are known in the art. In an additional embodiment, the polymer shell may be used to protect the polynucleotides in the core.

Core-shell nanoparticles for use with the mRNA of the present invention are described and may be formed by the methods described in U.S. Pat. No. 8,313,777 or International Patent Publication No. WO2013124867, the contents of each of which are herein incorporated by reference in their entirety.

In one embodiment, the polymer used with the formulations described herein may be a modified polymer (such as, but not limited to, a modified polyacetal) as described in International Publication No. WO2011120053, the contents of which are herein incorporated by reference in its entirety.

In one embodiment, the formulation may be a polymeric carrier cargo complex comprising a polymeric carrier and at least one nucleic acid molecule. Non-limiting examples of polymeric carrier cargo complexes are described in International Patent Publications Nos. WO2013113326, WO2013113501, WO2013113325, WO2013113502 and WO2013113736 and European Patent Publication No. EP2623121, the contents of each of which are herein incorporated by reference in their entireties. In one aspect the polymeric carrier cargo complexes may comprise a negatively charged nucleic acid molecule such as, but not limited to, those described in International Patent Publication Nos. WO2013113325 and WO2013113502, the contents of each of which are herein incorporated by reference in its entirety.

In one embodiment, a pharmaceutical composition may comprise the mRNA of the invention and a polymeric carrier cargo complex.

As a non-limiting example, the core-shell nanoparticle may be used to treat an eye disease or disorder (See e.g. US Publication No. 20120321719, the contents of which are herein incorporated by reference in its entirety).

In one embodiment, the polymer used with the formulations described herein may be a modified polymer (such as, but not limited to, a modified polyacetal) as described in International Publication No. WO2011120053, the contents of which are herein incorporated by reference in its entirety.

### Methods of Treatment

In one embodiment, an effective amount of the mRNA as described herein is used to treat or prevent a medical disorder which is mediated by the presence of a Flavivirus, for example an infection. In one embodiment, a method is provided comprising administering an effective amount of the mRNA described herein to a human to treat a Flavivirus infection.

The invention is directed to a method of treatment of a Flavivirus infection, including possible future drug resistant and multidrug resistant forms of the Flavivirus and related disease states, conditions, or complications of a Flavivirus infection, including severe fever disease, encephalitis, meningitis, hemorrhagic fever disease, shock syndrome, hepatitis, persistent infection of the testes, and multi-organ disease.

Non-limiting examples of diseases caused by Flavivirus that can be treated by the therapeutic composition of the present invention include Dengue hemorrhagic fever and Dengue shock syndrome, Kyasanur Forest disease, Powassan disease, Wesselsbron disease, yellow fever hepatitis, Zika virus testes infection, and encephalitis caused by West Nile, Rio bravo, Rocio, Negishi, California encephalitis, central European encephalitis, Ilheus, Murray Valley, St. Louis, Japanese, Louping ill, and Russian spring-summer encephalitis virus.

In one embodiment, the Flavivirus to be treated is a tick-borne Flavivirus species. In one embodiment, the Flavivirus is a mammalian tick-borne Flavivirus species. Non-limiting examples of mammalian tick-borne Flavivirus species include Greek goat encephalitis virus (GGEV), Kadam virus (KADV), Krasnodar virus (KRDV), Mogiana tick virus (MGTV), Ngoye virus (NGOV), Sokuluk virus (SOKV), Spanish sheep encephalomyelitis virus (SSEV), and Turkish sheep encephalitis virus (TSE). In one embodiment, the mammalian tick-borne Flavivirus species is of the tick-borne encephalitis virus serocomplex, including but not limited to Absettarov virus, deer tick virus (DT), Gadgets Gully virus (GGYV), Karshi virus, Kyasanur Forest disease virus (KFDV), Alkhurma hemorrhagic fever virus (ALKV), Langat virus (LGTV), Louping ill virus (LIV), Omsk hemorrhagic fever virus (OHFV), Powassan virus (POWV), Royal Farm virus (RFV), and tick-borne encephalitis virus (TBEV). In one embodiment, the mammalian tick-borne Flavivirus species is a seabird tick-borne Flavivirus species including, but not limited to, Kama virus (KAMV), Meaban virus (MEAV), Saumarez Reef virus (SREV), and Tyuleniy virus (TYUV).

In one embodiment, the Flavivirus to be treated is a mosquito-borne Flavivirus species. In one embodiment, the mosquito-borne Flavivirus species is of the Aroa virus group including, but not limited to, Aroa virus (AROAV), Bussuquara virus (BSQV), Iguape virus (IGUV), and Naranjal virus (NJLV). In one embodiment, the mosquito-borne Flavivirus species is of the Dengue virus group including, but not limited to, Dengue virus (DENV) and Kedougou virus (KEDV). In one embodiment, the mosquito-borne Flavivirus species is of the Japanese encephalitis virus group including, but not limited to, Cacipacore virus (CPCV), Koutango virus (KOUV), Kunjin virus, Ilheus virus (ILHV), Japanese encephalitis virus (JEV), Murray Valley encephalitis virus (MVEV), Alfuy virus, St. Louis encephalitis virus (SLEV), Usutu virus (USUV), West Nile virus (WNV), and Yaounde viris (YAOV). In one embodiment, the mosquito-borne Flavivirus species is of the Kokobera virus group including, but not limited to, Kokobera virus (KOKV), New Mapoon virus (NMV), and Stratford virus (STRV). In one embodiment, the mosquito-borne Flavivirus species is of the Ntaya virus group including, but not limited to, Bagaza virus (BAGV), Baiyangdian virus (BYDV), Duck egg drop syndrome virus (DEDSV), Ilheus virus (ILHV), Israel turkey meningoencephalomyelitis virus (ITV), Jiangsu virus (JSV), Layer flavivirus, Ntaya virus (NTAV), Rocio virus (ROCV), Sitiawan virus (STWV), T'Ho virus, and Tenbusu virus (TMUV). In one embodiment, the mosquito-borne Flavivirus species is of the Spondweni virus group including, but not limited to, Spondweni virus (SPOV) and Zika virus (ZIKV). In one embodiment, the mosquito-borne Flavivirus species is of the Yellow fever virus group including, but not limited to, Banzi virus (BANV), Bamaga virus (BGV), Bouboui virus (BOUV), Edge Hill virus (EHV), Fitzroy river virus, Jugra virus (JUGV), Saboya virus (SABV), Sepik virus (SEPV), Uganda S virus (UGSV), Wesselsbron virus (WESSV), and Yellow fever virus (YFV).

Additional non-limiting examples of mosquito-borne Flavivirus species include Aedes flavivirus, Barkedji virus, Calbertado virus, Cell fusing agent virus, Chaoyang virus, Culex flavivirus, Culex theileri flavivirus, Culiseta flavivirus, Donggang virus, Ilomantsi virus, Kamiti River virus, Lammi virus, Marisma mosquito virus, Nounane virus, Nhumirim virus, Mienokoue virus, Panmujeom flavivirus, Spanish Culex flavivirus, Spanish Ochlerotatus flavivirus, and Quang Binh virus. Additional non-limiting examples of mosquito-borne flaviviruses include Batu cave virus, Bukulasa bat virus, Nanay virus, Rabensburg virus (RABV), and Sitiawan virus.

Additional non-limiting examples of Flavivirus species include: Tamana bat virus; members of the Entebbe virus group, including Entebbe bat virus (ENTV), Sokoluk virus, and Yokose virus (YOKV); members of the Modoc virus group, including Apoi virus (APOIV), Cowbone Ridge virus (CRV), Jutiapa virus (JUTV), Modoc virus (MODV), Sal Vieja virus (SVV), and San Perlita virus (SPV); and members of the Rio Bravo virus group, including Bukalasa bat virus (BBV), Carey Island virus (CIV), Dakar bat virus (DBV), Montana myotis leukoencephalitis virus (MMLV), Phnom Penh bat virus (PPBV), and Rio Bravo virus (RBV).

In one embodiment, a method is provided for treating a human with West Nile virus severe fever disease comprising administering an effective amount of an mRNA or its pharmaceutical composition as described herein.

In another embodiment, a method is provided for treating a human with Dengue hemorrhagic fever/shock syndrome comprising administering an effective amount of an mRNA or its pharmaceutical composition as described herein.

In another embodiment, a method is provided for treating a human with tick-borne or mosquito-borne flavivirus encephalitis or other nervous system manifestations comprising administering an effective amount of an mRNA or its pharmaceutical composition as described herein.

In another embodiment, a method is provided for treating a human with yellow fever virus induced hepatitis comprising administering an effective amount of an mRNA or its pharmaceutical composition as described herein.

In another embodiment, a method is provided for treating a human with yellow fever virus vaccine induced multi-organ disease comprising administering an effective amount of an mRNA or its pharmaceutical composition as described herein.

In another embodiment, a method is provided for treating a human with a persisting Zika virus infection in the testes comprising administering an effective amount of an mRNA or its pharmaceutical composition as described herein.

In some embodiments, the mRNA of the present invention can be administered in combination with an additional therapeutic agent. For example, the mRNA may be administered in combination with an additional therapeutic selected from: interferon-alpha2a; a helicase inhibitor or other small molecule virus inhibitor; an antisense oligodeoxynucleotide (S-ODN); an aptamer; a nuclease resistant ribozyme; an iRNA such as microRNA or siRNA; an antibody, partial antibody, or domain antibody for the virus; or a viral antigen or partial antigen that induces a host antibody response.

### EXPERIMENTAL EXAMPLES OF THE INVENTION

### Example 1. Expression of the Resistant Oas1b Protein in Three Lines of Human Cells from a Transfected Stabilized mRNA Reduces the Amount of Viral dsRNA in and Virus Yield from Infected Cells

Three cell lines, susceptible 3T3 mouse C57BL/6 embryo fibroblast cells, human hepatocyte Huh7 cells, and human lung A549 cells were grown on coverslips in 24 well plates and were each infected with the West Nile virus strain Eg101 at a MOI of 1. Six hours later, the cells were transfected with either 1 μg V5-Oas1b (V5-1b) mRNA or GFP mRNA. The cell culture media was harvested at 36 hours after infection and virus infectivity was assessed by plaque assay. Harvested culture media was serially diluted ten-fold and the dilutions were adsorbed onto confluent monolayers of BHK 21/W12 cells in 6 well plates for 1 hr at 37° C. in a 5% CO₂ atmosphere. After the incubation period, the virus inoculum was aspirated and the cells were washed with Hank's balanced salt solution and then overlaid with 2 ml/well of 1% SeaChem agarose (Bio-Whittaker Molecular Applications) mixed 1:1 with 2×MEM containing 2.5% fetal calf serum. The plates were incubated at 37° C. for 72 hours and then stained using 0.05% crystal violet in 10% ethanol. All samples were assayed in duplicate.

The intracellular double stranded (replicating) viral RNA levels were measured by an immunofluorescence assay. The cells on coverslips were fixed with 4% paraformaldehyde for 10 min, followed by permeabilization for 10 min with 0.1% Triton-X and then blocking with 5% horse serum at room temperature for 1 h. After incubation with rabbit anti-V5 antibody (Abcam, 1:500) and mouse anti ds-RNA antibody (English & Scientific Consulting Kft., 1:1000) overnight at 4° C., the cells were incubated with Alexa Fluor 488-donkey anti-rabbit antibody (Thermo Fisher Scientific, 1:400), Alexa Fluor 594 donkey anti-mouse antibody (Thermo Fisher Scientific, 1:400) and Hoechst 33342 (Thermo Fisher Scientific, 0.05%) for 1 h at room temperature. The cover slips were then mounted on a glass slide with Prolong Gold Antifade reagent (Invitrogen) and the cells were imaged with a Zeiss Axio Observer 1 microscope using a 40× oil emersion objective.

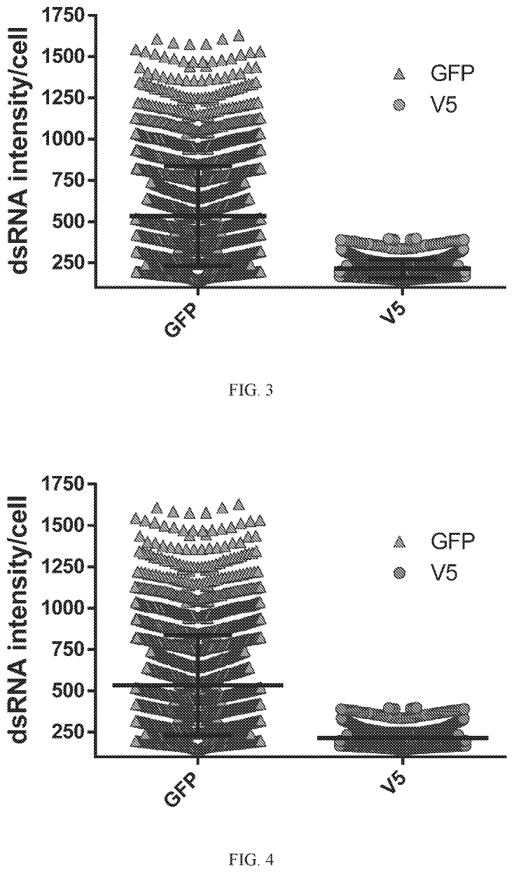

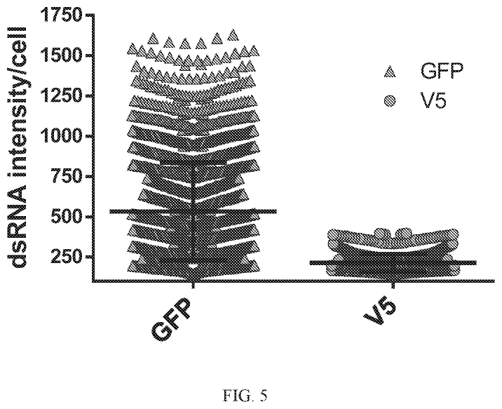

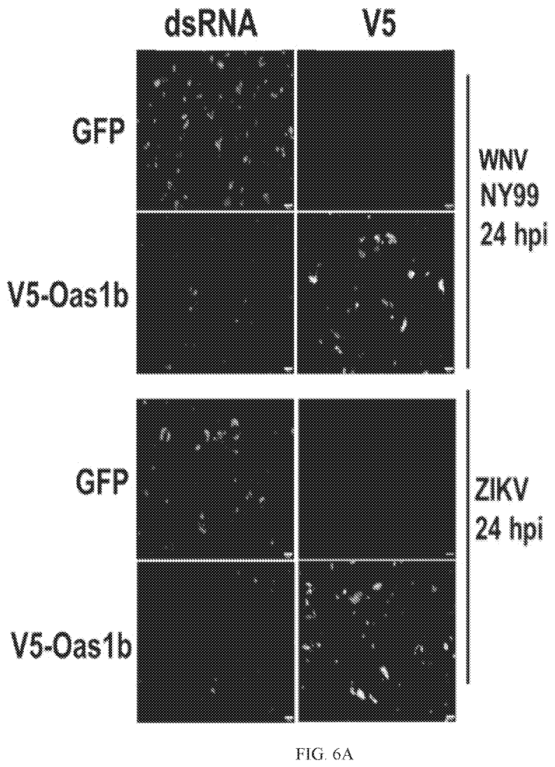

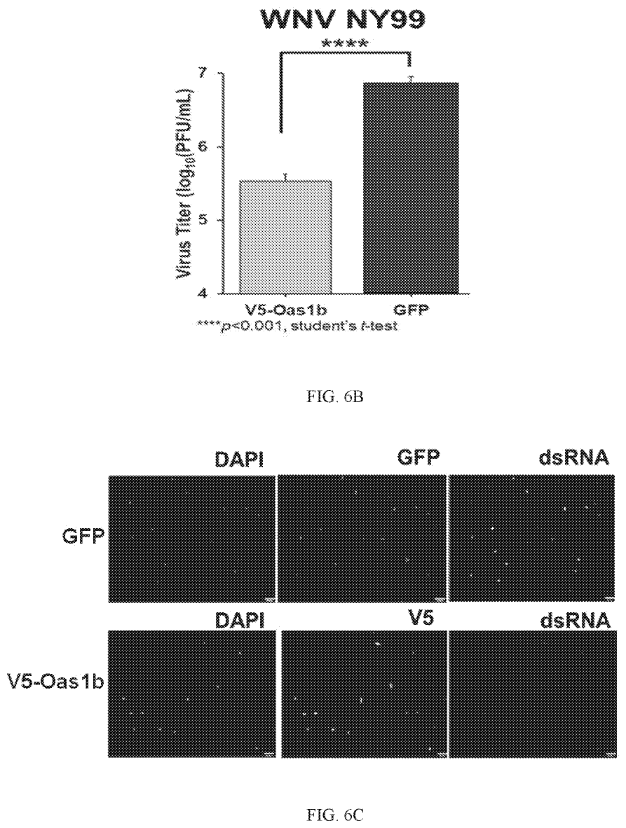

As shown in FIG. 2, the 36-hour virus yields (plaque forming units per ml) produced by each of the three types of cells transfected with the V5-Oas1b mRNA were compared to the virus yields produced by cells transfected with GFP mRNA. As shown in FIGS. 3-5, all three of the cell lines transfected with V5-Oas1b showed significant reductions in viral dsRNA levels compared to those in cells transfected with GFP mRNA.

### Example 2. Expression of the Resistant Oas1b Protein in Primary Human Astrocyte Cell Lines from a Transfected Stabilized mRNA Reduces the Amount of Viral dsRNA in and Virus Yield from Infected Cells

Primary human astrocytes were infected with West Nike virus (WNV), strain NY99, or Zika virus at a MOI of 2. After 6 hours, the cells were transfected with either 1.5 ug V5-Oas1b mRNA or GFP mRNA. At 24 hours after infection, cells were processed for indirect immunofluorescence assay (IFA) or culture fluids were harvested and used to determine virus yield by plaque assay.

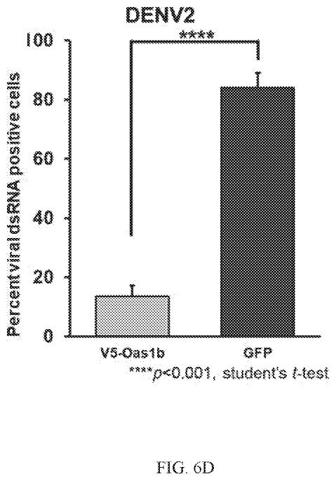

As shown in FIG. 6A, primary human astrocytes infected with West Nile virus and transfected with the Oas1b mRNA showed significant reductions in viral dsRNA levels compared to those cells transfected with the GFP control. Similar results were achieved in the Zika virus infected cells. Furthermore, as shown in FIG. 6B, the Oas1b mRNA treated primary human astrocyte cells showed significant reductions in plaque titers compared to those cells treated with GFP control. Statistical analysis was performed using the student's t-test. ****p<0.001.

### Example 3. Expression of the Resistant Oas1b Protein in Primary Human Monocyte Cell Lines from a Transfected Stabilized mRNA Reduces the Amount of Viral dsRNA in and Virus Yield from Infected Cells

Primary human monocytes were infected with Dengue virus, strain 2 at a MOI of 2. After 6 hours, the cells were transfected with either 1.5 ug V5-Oas1b mRNA or GFP mRNA. At 48 hours after infection, cells were processed for indirect immunofluorescence assay (IFA) or culture fluids were harvested and used to determine virus yield by plaque assay.

As shown in FIG. 6C, primary human monocytes infected with Dengue virus, strain 2 and transfected with the Oas1b mRNA showed significant reductions in viral dsRNA levels compared to those cells transfected with the GFP control. Furthermore, as shown in FIG. 6D, the Oas1b mRNA treated primary human monocyte cells showed significant reductions in plaque titers compared to those cells treated with GFP control. Statistical analysis was performed using the student's t-test. ****p<0.001.

### Example 4. Manufacture of mRNA for Expression of Resistant Oas1b

Coding regions are synthesized as DNA commercially. This DNA also contains a 5′ UTR with a Kozak sequence, a 3′ UTR, and extensions to allow for Gibson assembly. The DNA is cloned into a PCR amplified pMA7 vector through Gibson assembly using NEB Builder with 3× molar excess of insert. All reaction transcripts are gel purified prior to assembly reaction. Subsequent plasmids from each colony are Sanger sequenced to ensure desired sequence fidelity. Plasmids are linearized enzymatically overnight at 37° C. Linearized templates are purified by sodium acetate precipitation before being rehydrated with nuclease free water. In vitro transcription is performed overnight at 37° C. RNA product is treated with DNase I for 30 minutes to remove template and purified using lithium chloride precipitation. The RNA is heat denatured at 65° C. for 10 minutes before being capped with a Cap-1 structure enzymatically. Transcripts are next polyadenylated enzymatically. The mRNA is then purified by lithium chloride precipitation, treated with alkaline phosphatase, and purified again. Concentrations are measured using a Nanodrop.

1. A method for the treatment of an infection in a human subject caused by a Flavivirus comprising administering to the subject an effective amount of an mRNA comprising a coding region encoding Mus musculus resistant 2′-5′ oligoadenylate synthetase 1b (rOas1b).

2. The method of claim 1, wherein the mRNA coding region encodes the polypeptide of SEQ. ID. NO.: 1.

3. The method of claim 2, wherein the polypeptide of SEQ. ID. NO.: 1 has an amino acid substitution selected from the group consisting of A36S substitution, S45F substitution, R47Q substitution, V50G substitution, G63C substitution, T65A substitution, S83Y substitution, Q90R substitution, C103Y substitution, V105I substitution, C111F substitution, H118Q substitution, L151V substitution, P176L substitution, K181E substitution, S183L substitution, I184T substitution, R190Q substitution, R206H substitution, Q266R substitution, H277L substitution, Q278P substitution, D291V substitution, A299V substitution, I305V substitution, A322T substitution, S336P substitution, G347A substitution, M350T substitution, L354F substitution, and F36L substitution, or a combination thereof.

4. The method of claim 1, wherein the mRNA coding region encodes the polypeptide of SEQ. ID. NO.: 2, SEQ. ID. NO.: 3, or SEQ. ID. NO.: 4.

5-6. (canceled)

7. The method of claim 1, wherein the mRNA comprises the sequence of SEQ. ID. NO.: 42.

8. The method of claim 1, wherein the mRNA further comprises a human-derived or synthetic 5′ untranslated region (5′ UTR), human-derived or synthetic 3′ untranslated region (3′ UTR), or both a human-derived or synthetic 5′ UTR and a human-derived or synthetic 3′ UTR.

9. (canceled)

10. The method of claim 1, wherein the mRNA further comprises at least one chemical modification to an adenosine ribonucleoside, a cytidine ribonucleoside, a guanosine ribonucleoside, or a uridine ribonucleoside, or a combination thereof.

11. The method of claim 10, wherein the chemical modification comprises substituting one or more uridine ribonucleosides with N1-methyl-pseudouridine.

12. The method of claim 1, wherein the mRNA coding region is codon optimized.

13. The method of claim 1, wherein the infection is caused by a Flavivirus selected from the group consisting of West Nile virus, yellow fever virus, tick-borne encephalitis virus, Dengue virus, Japanese encephalitis virus, Powassan virus, and Zika virus.

14. The method of claim 1, wherein the mRNA is administered by direct injection into the brain, direct injection into the testes, intrathecal injection, intravenous injection, or targeted ultrasound for delivery to the brain.

15-20. (canceled)

21. The method of claim 1, wherein the mRNA comprises a 5′ untranslated region (5′ UTR) comprising a sequence selected from the group consisting of SEQ. ID. NOS.: 6-23,
a 3′ untranslated region (3′ UTR) comprising a sequence selected from the group consisting of SEQ. ID. NOS.: 24-41, or
a 5′ UTR comprising a sequence selected from the group consisting of SEQ. ID. NOS.: 6-23 and a 3′ UTR comprising a sequence selected from the group consisting of SEQ. ID. NOS.: 24-41.

22. (canceled)

23. An mRNA comprising: a coding region encoding a polypeptide of SEQ. ID. NO.: 1, SEQ. ID. NO.: 2, SEQ. ID. NO.: 3, or SEQ. ID. NO.: 4; a human-derived or synthetic 5′ untranslated region (UTR) operably linked to the 5′ end of the coding region; and a human-derived or synthetic 3′ UTR operably linked to the 3′ end of the coding region.

24. The mRNA of claim 23, wherein the polypeptide of SEQ. ID. NO.: 1 has an amino acid substitution selected from the group consisting of A36S substitution, S45F substitution, R47Q substitution, V50G substitution, G63C substitution, T65A substitution, S83Y substitution, Q90R substitution, C103Y substitution, V105I substitution, C111F substitution, H118Q substitution, L151V substitution, P176L substitution, K181E substitution, S183L substitution, I184T substitution, R190Q substitution, R206H substitution, Q266R substitution, H277L substitution, Q278P substitution, D291V substitution, A299V substitution, I305V substitution, A322T substitution, S336P substitution, G347A substitution, M350T substitution, L354F substitution, and F36L substitution, or a combination thereof.

25. The mRNA of claim 23, wherein the 5′ UTR is selected from the group consisting of SEQ. ID. NOS.: 6-23,
wherein the 3′ UTR is selected from the group consisting of SEQ. ID. NOS.: 24-41, or
wherein the 5′ UTR is selected from the group consisting of SEQ. ID. NOS.: 6-23 and the 3′ UTR is selected from the group consisting of SEQ. ID. NOS.: 24-41.

26-35. (canceled)

36. The RNA of claim 23, wherein the coding region encodes a polypeptide of SEQ. ID. NO.: 4, wherein the 5′ UTR is SEQ. ID. NO.: 6, and wherein the 3′ UTR is SEQ. ID. NO.: 24.

37. The mRNA of claim 23 further comprising at least one chemical modification to an adenosine ribonucleoside, a cytidine ribonucleoside, a guanosine ribonucleoside, or a uridine ribonucleoside, or a combination thereof.

38. The mRNA of claim 37, wherein the chemical modification comprises substitution of one or more uridine ribonucleosides with N1-methyl-pseudouridine.

39. The mRNA of claim 23, wherein the mRNA is codon optimized.

40. The mRNA of claim 23 further comprising a 5′ terminal cap operably linked to the 5′ end of the mRNA.

41. The mRNA of claim 40, wherein the 5′ terminal cap has a cap-1 structure.

42. The mRNA of claim 23 further comprising a 3′ poly(A) tail operably linked to the 3′ end of the mRNA.

43. A pharmaceutical composition comprising an mRNA of claim 23 formulated within a delivery vehicle.

44. The pharmaceutical composition of claim 43, wherein the delivery vehicle is a liposome, a lipoplex, a lipid nanoparticle, a polymer, or a polymeric nanoparticle.

45. The pharmaceutical composition of claim 44, wherein the liposome comprises a 3:1 mixture of 2,3-dioleyloxy-N-[2(sperminecarboxamido)ethyl]-N,N-dimethyl-1-propanaminium trifluoroacetate (DOSPA) and dioleylphosphatidylethanolamine (DOPE).

46-48. (canceled)

49. The pharmaceutical composition of claim 48, wherein the polymer is modified poly(ethyleneimine) (PEI).

50-53. (canceled)
